(19) United States
(12) Patent Application Publication
(10) Pub. No.: US 2017/0216426 A1
(43) Pub. Date: Aug. 3, 2017

(54) COMPOSITIONS AND METHODS FOR TRANSIENT IMMUNE RESPONSE MODULATION DURING VACCINATION

(71) Applicant: DUKE UNIVERSITY, Durham, NC (US)

(72) Inventors: Barton F. HAYNES, Durham, NC (US); Garnett KELSOE, Durham, NC (US); Masayuki KURAOKA, Durham, NC (US); M. Anthony MOODY, Durham, NC (US); Hua-Xin LIAO, Durham, NC (US); Laurent VERKOCZY, Durham, NC (US)

(21) Appl. No.: 15/514,789

(22) PCT Filed: Sep. 28, 2015
(86) PCT No.: PCT/US2015/052660

§ 371 Date: Mar. 27, 2017

Related U.S. Application Data
(60) Provisional application No. 62/056,583, filed on Sep. 28, 2014.

(51) Int. Cl. A61K 39/21; C07K 16/28; A61K 31/4706; A61K 38/17

(52) U.S. Cl. CPC A61K 39/21; A61K 38/177; C07K 16/2866; A61K 31/4706; C12N 2740/16134; A61K 2039/55555

(57) ABSTRACT

In certain aspects the invention provides a selection of HIV-1 envelopes suitable for use as immunogens, and methods of using these immunogens to induce neutralizing antibodies. In certain embodiments, the immunogens are designed to trimerize. In other embodiments, the immunogenic compositions and methods comprise at least one agent for transient immune response modulation during vaccination.

This application claims the benefit of U.S. Application Ser. No. 62/056,583 filed Sep. 28, 2014, the entire content of which application is herein incorporated by reference.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### STATEMENT OF FEDERALLY FUNDED RESEARCH

This invention was made with government support under Center for HIV/AIDS Vaccine Immunology-Immunogen Design grant UM1-AI100645 and AI 067854 from the NIH, NIAID, Division of AIDS, and NIH grants AI24335 and AI56363. The government has certain rights in the invention.

### FIELD OF THE INVENTION

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to immunization methods for inducing such broadly neutralizing anti-HIV-1 antibodies using such compositions and agents which transiently modulate the host immune response.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain aspects the invention provides methods and compositions of inducing an immune response in a subject in need thereof comprising administering a composition comprising an HIV-1 immunogen, or a combination of several HIV-1 immunogens, and a first immunomodulatory agent in an amount sufficient to induce an immune response. The immunomodulatory agent transiently modulates the subject's immune response during an immunization schedule. In certain embodiments, where the immunogenic composition or immunogen comprises CD40L, no other immunomodulatory agent is administered. In certain embodiments, the induced immune response comprises induction of broad neutralizing antibodies (bnAbs) against HIV-1 envelope.

In certain embodiments, the HIV-1 immunogen is HIV-1 envelope, a fragment thereof, or a peptide derived from HIV-1 envelope. In certain embodiments, the immunogen against the HIV-1 envelope is designed as a fusion protein which comprises a trimerization domain. In certain embodiments, the immunogen against the HIV-1 envelope is designed as a fusion protein which comprises a CD40L. In certain embodiments, the compositions comprise an immunogen and CD40L.

In certain embodiments, the immune response modulated by the methods and compositions of the invention is a humoral immune response. In certain embodiments, the immune response is modulated during immunization against an HIV-1 virus, e.g. HIV-1 envelope.

In certain embodiments, the modulation includes PD-1 blockade; T regulatory cell depletion; CD40L hyperstimulation; or soluble antigen administration, wherein the soluble antigen is designed such that the soluble agent eliminates B cells targeting dominant epitopes.

In certain embodiments, the humoral immune response comprises the induction of a broad neutralizing antibody (bNAb) against HIV-1 envelope. In certain embodiments, an HIV-1 immunogen induce a CD4bs bNAb, a V3-glycan bNAb, a V1V2-glycan bNAb, a gp41 bNAb, or a combination of broadly neutralizing antibodies. In certain embodiments, the induction of bnAbs lineages in a subject is detected by any suitable method including but not limited to neutralization assays against HIV-1 virus, binding assays to detect binding to certain antigens, sequence analyses methods to detect nucleotide sequences of certain bnAbs.

In certain embodiments, the agent is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355|Foxo1 Inhibitor, AS1842856—Calbiochem; Gleevac, an anti-CD25 antibody, an anti-CCR4 Ab, a small molecule antagonist of CCR4 such as SP46, SP50 or CB20 (Davies M N et al PLOS ONE 4: e8084, 2009; Pere, H Blood 118: 4853, 2011), or an agent which binds to a B cell receptor for a dominant HIV-1 envelope epitope, or any combination thereof. In certain embodiments, the agent, for example chloroquine, is administered before and ˜3-7 days after each immunization. In certain embodiments, the agent is a CQ derivative for example but not limited to hydroxychloroquine, primaquine diphosphate (PQ) and amodiaquine dihydrochloride dihydrate (AQ) (see Bo\ddot{} nsch C, Kempf C, Mueller I, Manning L, Laman M, et al. (2010) Chloroquine and Its Derivatives Exacerbate B19V-Associated Anemia by Promoting Viral; Replication. PLoS Negl Trop Dis 4(4): e669. doi:10.1371/journal.pntd.0000669; chloroquine phosphate, hydrochloroquine, and enantiomers and any other derivative (See U.S. Pat. No. 6,417,177 B1). In certain embodiments, the agent is clodronate, or any other bisphosphonate. In certain embodiments, the first agent, for example chloloquine, is administered for 15, 14, 13, 12, 11, 10, 9, 8, 7, 6, 5, 4, 3, 2 or 1 days before each immunization.

In certain embodiments, the methods further comprise administering a second immunomodulatory agent. In certain embodiments, the second and first immunomodulatory agents are different. In non-limiting embodiments, the immunostimulatory agents, target the bone marrow (first) and peripheral (second) immune system immune tolerance checkpoints, whereby these agents very transiently disrupt immune mechanisms of host tolerance that block the induction and/or development of autoreactive or otherwise disfavored B cells with traits of long heavy chain complementarity determining region 3 (HCDR3s), polyreactivity or autoreactivity, and high levels of somatic mutations.

In certain embodiments, the second agent is anti-CD25 or anti-CCR4 antibody. In certain embodiments, the anti-CD25 antibody is administered after each immunization (in certain embodiments, anti-CD25 antibody is administered for about 5-7, 1, 2, 3, 4, 5, 6, 7, 8, 9, or 10 days either before or after each immunization. Administering CD25 Abs days after immunization is targeted to disrupting T regulatory control of germinal center disfavored B cell clonal lineage expansion. CD25 antibodies, including humanized, chimeric and human antibodies are known in the art. In a non-limiting embodiment, the CD25 antibody is basiliximab. In a non-limiting embodiment, the CD25 antibody is daclizumab (Zenapax). In certain embodiments, CD25 antibody is administered only in combination with another immunomodulatory agent. In certain embodiments, CD25 antibody is administered as one of the agents in an immunization schedule which includes at least another immunomodulatory agent. In certain embodiments, the immunomodulatory agents are administered sequentially. On certain embodiments one of the agents is administered before administering of an immunogen.

In certain embodiments the first and/or second agent is administered before immunization with the HIV-1 immunogen. In certain embodiments the first and/or second agent are administered multiple times before and/or following immunization with the HIV-1 immunogen.

In certain embodiments, the HIV-1 immunogen is administered as a nucleic acid, a protein or any combination thereof. In certain embodiments, the nucleic acid encoding the HIV-1 immunogen is operably linked to a promoter inserted in an expression vector. In certain embodiments, the protein is recombinant.

In certain embodiments, the immunogenic composition is administered as a prime, a boost, or both. In certain embodiments, the composition is administered as a multiple boosts.

In certain embodiments, the nucleic acid form of the Env is administered simultaneously with the protein form of the Env immunogen.

In certain embodiments, the immunogens are formulated in any suitable adjuvant.

In certain embodiments, the immunogens are HIV-1 envelopes that are administered as a nucleic acid, a protein or any combination thereof. In certain embodiments, the nucleic acid encoding the envelope is operably linked to a promoter inserted in an expression vector. In certain embodiments, the protein is recombinant.

In certain embodiments, the envelopes are administered as a prime, a boost, or both. In certain embodiments, the envelopes, or any combinations thereof are administered as a multiple boosts. In certain embodiments, the compositions and method further comprise an adjuvant. In certain embodiments, the HIV-1 envelopes are provided as nucleic acid sequences, including but not limited to nucleic acids optimized for expression in the desired vector and/or host cell. In other embodiments, the HIV-1 envelopes are provided as recombinantly expressed protein.

In certain embodiments, the invention provides compositions and method for induction of immune response, for example cross-reactive (broadly) neutralizing Ab induction. In certain embodiments, the methods use compositions comprising “swarms” of sequentially evolved envelope viruses that occur in the setting of bnAb generation in vivo in HIV-1 infection.

In certain aspects the invention provides compositions comprising a selection of HIV-1 envelopes or nucleic acids encoding these envelopes, for example but not limited to, as described herein. In certain embodiments, these compositions are used in immunization methods as a prime and/or boost, for example but not limited to, as described herein.

In certain embodiments, the compositions contemplate nucleic acid, as DNA and/or RNA, or protein immunogens either alone or in any combination. In certain embodiments, the methods contemplate genetic, as DNA and/or RNA, immunization either alone or in combination with envelope protein(s).

In certain embodiments the nucleic acid encoding an envelope is operably linked to a promoter inserted in an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides a nucleic acid comprising any one of the nucleic acid sequences of invention. In certain aspects the invention provides nucleic acids formulated with polyamines to facilitate cell uptake. In certain aspects the invention provides a nucleic acid consisting essentially of any one of the nucleic acid sequences of invention. In certain aspects the invention provides a nucleic acid consisting of any one of the nucleic acid sequences of invention. In certain embodiments the nucleic acid of invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising a combination of one nucleic acid sequence encoding any one of the polypeptides of the invention. In certain embodiments, combining DNA and protein gives higher magnitude of ab responses. See Pissani F. Vaccine 32: 507-13, 2013; Jalah R et al PLoS One 9: e91550, 2014.

In certain embodiments, the compositions and methods employ an HIV-1 envelope as polypeptide instead of a nucleic acid sequence encoding the HIV-1 envelope. In certain embodiments, the compositions and methods employ an HIV-1 envelope as polypeptide, a nucleic acid sequence encoding the HIV-1 envelope, or a combination thereof. The envelope can be a gp160, gp150, gp140, gp120, gp41, N-terminal deletion variants as described herein, cleavage resistant variants as described herein, or codon optimized sequences thereof. The polypeptide of the inventions can be a trimer. The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the polypeptides and nucleic acids of the invention are suitable for use as an immunogen, for example to be administered in a human subject.

### BRIEF DESCRIPTION OF THE DRAWINGS

To conform to the requirements for PCT patent applications, many of the figures presented herein are black and white representations of images originally created in color. In the below descriptions and the examples, the colored images are described in terms of its appearance in black and white.

FIG. 1 shows sequences of a selection of ten envelopes (“Production10”) (derived from African HIV infected individual CH505). In certain embodiments these envelopes are gp120s or gp140s proteins. In other embodiments these envelopes are designed to be trimers. In other embodiments these envelopes are gp145s or gp160s as DNAs. The nucleotide sequences for the following GP120 DNA constructs are shown: >HV1300532_v2, CH505.M6D8gp120 (SEQ ID NO.: 15), >HV1300537_v2, CH505.M11D8gp120 (SEQ ID NO.: 16), >HV1300556_v2, CH505w020.14D8gp120 (SEQ ID NO.: 17), >HV1300578_v2, CH505w030.28D8gp120 (SEQ ID NO.: 18), >HV1300574_v2, CH505w030.21D8gp120 (SEQ ID NO.: 19), >HV1300583, CH505w053.16D8gp120 (SEQ ID NO.: 20), >HV1300586, CH505w053.31D8gp120 (SEQ ID NO.: 21), >HV1300595, CH505w078.33D8gp120 (SEQ ID NO.: 22), >HV1300592, CH505w078.15D8gp120 (SEQ ID NO.: 23), >HV1300605, CH505w100.B6D8gp120 (SEQ ID NO.: 24). The amino acid sequences of the production 10 CH505 Δ8gp120 are shown: >HV1300532_v2, CH505.M6D8gp120 (SEQ ID NO.: 25), >HV1300537_v2, CH505.M11D8gp120 (SEQ ID NO.: 26), >HV1300556_v2, CH505w020.14D8gp120 (SEQ ID NO.: 27), >HV1300578_v2, CH505w030.28D8gp120 (SEQ ID NO.: 28), >HV1300574_v2, CH505w030.21D8gp120 (SEQ ID NO.: 29), >HV1300583, CH505w053.16D8gp120 (SEQ ID NO.: 30), >HV1300586, CH505w053.31D8gp120 (SEQ ID NO.: 31), >HV1300595, CH505w078.33D8gp120 (SEQ ID NO.: 32), >HV1300592, CH505w078.15D8gp120 (SEQ ID NO.: 33), >HV1300605, CH505w100.B6D8gp120 (SEQ ID NO.: 34). The nucleotide sequences for the following Gp145 DNA constructs are shown: >HV1300657 (SEQ ID NO.: 35), >HV1300662 (SEQ ID NO.: 36), >HV1300635 (SEQ ID NO.: 37), >HV1300636 (SEQ ID NO.: 38), >HV1300689 (SEQ ID NO.: 39), >HV1300696 (SEQ ID NO.: 40), >HV1300638 (SEQ ID NO.: 41), >HV1300705 (SEQ ID NO.: 42), >HV1300639 (SEQ ID NO.: 43), >HV1300714 (SEQ ID NO.: 44). The nucleotide sequences for the following Gp160 constructs are shown: >CH505.M6 gp160 (SEQ ID NO.: 45), >CH505.M11 gp160 (SEQ ID NO.: 46), >CH505w020.14 160 (SEQ ID NO.: 47), >CH505w030.28 gp160 (SEQ ID NO.: 48), >CH505w030.21 160 (SEQ ID NO.: 49), >CH505w053.16 gp160 (SEQ ID NO.: 50), >CH505w053.31 160 (SEQ ID NO.: 51), >CH505w078.33 gp160 (SEQ ID NO.: 52), >CH505w078.15 gp160 (SEQ ID NO.: 53), >CH505w100.B6 gp160 (SEQ ID NO.: 54). The following GP160 amino acid sequences are shown: >CH505.M6 gp160 (SEQ ID NO.: 55), >CH505.M11 gp160 (SEQ ID NO.: 56), >CH505w020.14 gp160 (SEQ ID NO.: 57), >CH505w030.28 gp160 (SEQ ID NO.: 58), >CH505w030.21 gp160 (SEQ ID NO.: 59), >CH505w053.16 gp160 (SEQ ID NO.: 60), >CH505w053.31 gp160 (SEQ ID NO.: 61), >CH505w078.33 gp160 (SEQ ID NO.: 62), >CH505w078.15 gp160 (SEQ ID NO.: 63), >CH505w100.B6 gp160 (SEQ ID NO.: 64).

FIG. 2A shows an envelope V1V2 peptide and its glycosylation. FIG. 2B shows the double alanine substituted mutant V1V2 peptide. It makes up gp120 positions 165-182, and has alanine substitutions at L179 and I181. A244 L179A I181A: LRDKKQKVHALFYKADAV—it has N terminal acylation and C terminal amidation.

FIG. 3A shows an envelope V3 peptide and its glycosylation. FIG. 3B shows the sequence for the aglycone V3 peptide of FIG. 3A.

FIG. 4 shows designs of HIV-1 envelopes with trimerization domain, and immune modulating (e.g. CD40L) domain.

FIG. 5 shows designs of HIV-1 MPER peptide and immune modulating (e.g. CD40L) domain. The MPER peptides have any one of the following sequences:

MPER656.oriNEQELLELDKWASLWNWFNITNWLWYIK (SEQ ID NO.: 1) original

MPER656.1 NEQDLLALDKWASLWNWFDISNWLWYIK (SEQ ID NO.: 2)

MPER656.2 NEKDLLALDSWKNLWNWFSITKWLWYIK (SEQ ID NO.: 3)

MPER656.3 NEQELLALDKWNNLWSWFDITNWLWYIR (SEQ ID NO.: 4)

MPER656.ori-anchor NEQELLELDKWASLWNWFNITNWLWYIK-GTH1 (SEQ ID NO.: 5) original

MPER656.1-anchor NEQDLLALDKWASLWNWFDISNWLWYIK-GTH1 (SEQ ID NO.: 6)

MPER656.2-anchor NEKDLLALDSWKNLWNWFSITKWLWYIK-GTH1 (SEQ ID NO.: 7)

MPER656.3-anchor NEQELLALDKWNNLWSWFDITNWLWYIR-GTH1 (SEQ ID NO.: 8)

GTH1 sequence is YKRWIILGLNKIVRMYS (SEQ ID NO.: 9).

FIG. 6A shows sequences of a selection of four CH505 envelopes: >CH505w000.TFgp160 (SEQ ID NO.: 65), >CH505w053.16gp160 (SEQ ID NO.: 66), >CH505w078.33gp160 (SEQ ID NO.: 67), >CH505w100.B6gp160 (SEQ ID NO.: 68).

FIG. 6B shows the sequence of CAP206 6m envelope: >6mo_B6 (SEQ ID NO.: 69), >6mo_B6 (SEQ ID NO.: 70).

FIG. 6C shows sequences of a selection of ten early CH505 envelopes: >CH505M11gp160 (SEQ ID NO.: 71), >CH505w004.03gp160 (SEQ ID NO.: 72), >CH505w020.14gp160 (SEQ ID NO.: 73), >CH505w030.28gp160 (SEQ ID NO.: 74), >CH505w30.12 (SEQ ID NO.: 75), >CH505w020.2 (SEQ ID NO.: 76), >CH505w030.10gp160 (SEQ ID NO.: 77), >CH505w078.15gp160 (SEQ ID NO.: 78), >CH505w030.19gp160 (SEQ ID NO.: 79), >CH505w030.21gp160 (SEQ ID NO.: 80).

FIGS. 7A-7D show that AID mRNA expression in immature/T1 B cells is synergistically elevated by co-activation with CpG and anti-μ, through phospholipase-D activation, intracellular acidification, and MyD88. (FIG. 7A) AID mRNA expression in immature/T1 B cells (n=8-15) cultured for 24 h in the presence of indicated stimuli. Splenic GC B cells (n=4) from immunized mice. AID mRNA levels in B6 immature/T1 B cells stimulated with CpG or CpG+anti-μ in the presence of various concentrations of n-butanol (FIG. 7B; v/v, n=4) or chloroquine (FIG. 7C; μg/ml, n=3-12), or FIG. 7D, in Myd88^{-/-} immature/T1 B cells before (n=13) and after culture (n=4). Each point represents an individual mouse and determination. *P<0.05, **P<0.01, ***P<0.001.

FIGS. 8A-8B show that autoreactive immature/T1 B cells are enriched in Myd88^{-/-} mice. Single immature/T1 B (FIG. 8A) or MF B (FIG. 8B) cells isolated from B6 (n=5) and Myd88^{-/-} (n=3) mice were grown in N-cultures, and culture supernatants were analyzed for DNA reactivity [shown as DNA avidity indices (anti-DNA IgG/total IgG)] by ELISA. Each point represents an individual culture and determination. ***P<0.001; horizontal bars, geometric mean.

FIGS. 9A-9D show that development of autoreactive immature/T1 B cells are augmented in the absence of Myd88. (FIG. 9A) Representative flow diagrams for IgM/IgD expression by bone marrow cells of indicated mouse strains. (FIGS. 9B-9D) Absolute cell numbers of indicated B-cell compartments in bone marrow of B6.Myd88^{+/+} (n=15), B6.Myd88^{-/-} (n=6), B1-8 (n=9), 3H9 (n=29), 3H9.Myd88^{-/-} (n=12), 3H9.Aicda^{+/-} (n=5), 3H9.Myd88^{+/-} (n=7), 3H9.Aicda^{+/-} Myd88^{+/-} (n=18), 3H9.Aicda^{-/-} (n=10), and 3H9.BCL2-Tg mice (n=7). (FIG. 9D) Absolute cell numbers of the indicated B-cell compartments of 3H9.Aicda^{-/-} ,3H9.Myd88^{-/-}, and 3H9.BCL2-Tg mice (filled circles) are normalized to those of 3H9 mice (filled triangles). *P<0.05, * *P<0.01, ***P<0.001; error bars, s.e.m.

FIGS. 10A-10E show that intracellular acidification is required for central B-cell tolerance. (FIG. 10A) representative flow plots for IgM/IgD expression by B220^{lo}CD93⁺CD43⁻CD23⁻ small bone marrow lymphocytes; (FIG. 10B) ratio of immature/T1 B cells over small pre-B cells in B1-8 (n=9), and PBS-treated (n=7) and chloroquine-treated (n=5) 3H9 mice. FIGS. 10C-10E show single immature/T1 B cells isolated from control- (n=6), chloroquine-treated (n=5) 3H9, and Myd88^{-/-} 3H9 (n=2) mice were grown in N-cultures, and culture supernatants were analyzed for DNA reactivity by ELISA. (FIG. 10C) DNA avidity indices of individual immature/T1 B-cell cultures (n=206, 186, and 64 for control-, chloroquine-treated 3H9, and Myd88^{-/-}3H9, respectively). ***P<0.001; horizontal bars, median. (FIG. 10D) DNA avidity indices shown in FIG. 10C were compartmentalized by binning into 2-fold intervals. (FIG. 10E) Frequency of higher DNA avidity immature/T1 B cells (DNA avidity index ≧0.178, average DNA avidity index of control 3H9 immature/T1 B cells). *P<0.05; error bars, s.e.m.

FIGS. 11A-11B show the synergistic increase of AID expression by co-activation with CpG and anti-μ delays in splenic MF B cells. AID mRNA expressions in splenic MF B cells (n=3-8) cultured in the presence of indicated stimuli for 24 h (FIG. 11A), or cultured in the presence of CpG (open) or CpG and anti-μ (filled) stimulations for up to 72 h (FIG. 11B) are shown. AID levels in splenic GC B cells (n=4) are indicated. Each point represents an individual mouse and determination. *P<0.05, **P<0.01.

FIGS. 12A-12D show that Myd88 is required for central B-cell tolerance. Single immature/T1 B cells (FIGS. 12A, 12C) and MF B cells (FIGS. 12B, 12D) were sorted from bone marrow and spleen, respectively, from B6 (n=5) and Myd88^{-/-} (n=3) mice. These cells were expanded in N-cultures, and culture supernatants were analyzed for the presence of total- and anti-DNA IgG. In FIGS. 12A and 12B, 643-944 IgG samples were obtained from each compartment (indicated). Myd88^{-/-} B cells produced significantly lower quantities of IgG in N-cultures. In FIGS. 12C and 12D, anti-DNA avidity indices for all samples that contain 1-3 μg/ml IgG were compared between B6 and Myd88^{-/-} mice. Results are compatible with data in FIGS. 8A-8B where all IgG⁺ samples were included in analysis. ***P<0.001; horizontal bars, geometric mean.

FIG. 13 shows the effects of VDJ knock-in alleles on B-cell development. Absolute cell numbers of indicated B-cell compartments in the bone marrow of B1-8 VDJ knock-in mice (grey; n=9) were normalized to those of B6 counterparts (black; n=18). CD43⁺ pre-pro-B/pro-B cells between B6 and B1-8 mice are comparable, while large pre-B (L-pre-B), small pre-B (S-pre-B) and immature/transitional-1 (imm/T1) B cell numbers in B1-8 mice are significantly decreased to 43%, 43% and 69%, respectively. Mature B cell numbers are comparable between B6 and B1-8 mice. **, P<0.01; ***, P<0.001; error bars, s.e.m.

FIGS. 14A and 14B show that Chloroquine partially rescues B-cell development in 3H9 Mice. FIG. 14A shows the scheme in which 3H9 mice were treated with chloroquine and assessed B-cell development in these mice. FIG. 14B shows decreased immature/T1 B cells development in 3H9 mice are partially restored when mice were given chloroquine.

FIG. 15 shows that Chloroquine rescues Immature/T1 3H9 B cells that avidly bind DNA. Individual immature/T1 B cells from chloroquine-treated 3H9 mice bind DNA more avidly than those from control 3H9 mice. Right panel shows distribution of DNA avidity indices, which are relative values against standard anti-DNA mAb. The diamonds (connected by dotted line) correspond to B6 immature/T1 B cells, which show broader distributions with relatively lower avidity cohorts. The squares (connected by black line) correspond to 3H9 mice and show the distribution shifting toward higher avidity cohorts and becoming more uniform. The circles (connected by medium grey line) correspond to chloroquine injections that resulted in further shift toward higher avidity cohorts.

FIG. 16 shows that Chloroquine augments B-Cell development and maturation in 2F5 dKI mice which were treated with chloroquine for one week. This figure shows that chloroquine augments B-cell development in 2F5 dKI mice, and especially that it increases splenic mature B-cell compartments. These results provide use of chloroquine in vaccination strategies against HIV-1.

FIG. 17 shows that Chloroquine treatment suppresses humoral responses for <1 week.

FIG. 18 shows that CD25 Ab (PC61) reduces T_{reg} numbers by half without effect on T_{H}, T_{FH}, or T_{Freg}.

FIGS. 19A-19B show that CD25 Ab (PC61) lowers T_{reg} numbers by 50% for >14 days.

FIGS. 20A-20B show that injections of chloroquine release 2F5 dKI B cells from tolerizing deletion. FIG. 20A shows the treatment schedule. FIG. 20B shows B-cell numbers in Spleen of 2F5 dKI mice after immunization/treatments.

FIG. 21 shows that more HIV-1 chronics with BnAbs have positive assay for the Sm autoantigen compared to chronics with no BnAbs.

FIG. 22 shows by Illumina MiSeq Monitoring that a macaque clonal lineage of 2F5 VH Genes with all key traits of the human 2F5 BnAb disappeared over time.

FIG. 23 shows schematic representation of Vaccination Transient Immune Modulation (VTIM).

FIG. 24 shows that 2F5 mAb recognition of MPER is intact when co-anchored on liposomes with CD40L.

FIG. 25 shows set up for measuring bioactivity of CD40L anchored on liposomes. HEK-Blue CD40L cells (Invivogen) were used to measure the bioactivity of CD40L through the secretion of embryonic alkaline phosphatase (SEAP) upon NF-κB activation following CD40 stimulation.

FIGS. 26A-26C show that conjugation of CD40L to liposomes enhances CD40 triggering.

FIGS. 27A-27C show that HIV-1 gp41 MPER antibodies 2F5 and 4E10 bound strongly to CD40L-MPER656 liposomes.

FIG. 28 shows binding of antibody 2F5 to MPER656 liposomes with mouse-CD40L.

FIG. 29 shows activation of human CD40 expressing HEK blue cells by CD40L-MPER656 liposome. The line and circle designated (1) correspond to His6-hCD40L-MPER656 liposomes. The line and circle designated (2) correspond to His10-GCN4-L11-hCD40L-MPER656 liposomes. The line and circle designated (3) correspond to IgL-GCN4-L11-CD40L-His10-MPER656 liposomes.

FIG. 30 shows activation of human CD40 expressing HEK blue cells by CH505 gp120-GCN4-CD40L constructs. Both the Env constructs (with and without His tag) were active. Liposome conjugation did not enhance the activity of His tagged CH505 gp120-GCN4-CD40L construct. The Env without CD40L is not active showing that the CD40 activation by these constructs is CD40L mediated. The line and circle designated (1) correspond to CH505 gp120-GCN4-hCD40L. The line and circle designated (2) correspond to CH505 gp120-GCN4-hCD40L-10His. The line and circle designated (3) correspond to CH505 gp120-GCN4-hCD40L-10His liposomes. The line and circle designated (4) correspond to CH505 gp120-GCN4.

### DETAILED DESCRIPTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

About 50% of chronically infected individuals have plasma antibodies that neutralize ˜50% of tier 2 HIV strains (Hraber, P, Montefiori D et al. AIDS 28: 163, 2014). The question then is why when animals or humans are immunized with antigenic Envs, none of them make bnAbs—i.e. so far 0% of uninfected subjects make bnAbs following antigenic Env vaccination.

It appears that the HIV must be affecting the host to allow bnAbs to emerge. HIV induces autoimmune phenomena/disease syndromes. For example, about 50% of untreated HIV infected individuals will have either plasma autoantibodies (anti-CL, ANA, anti-DNA etc.) or have a frank autoimmune disease. Other diseases associated with HIV infection are SLE, Myasthenia gravis, Immune thrombocytopenia, Vasculitis. HIV infection—˜50% complicated with autoimmune syndromes and serologies (Brit. Journal. Haematol. 65: 495, 1987; Clin. Immunol. Immunopathol. 58: 163, 1991).

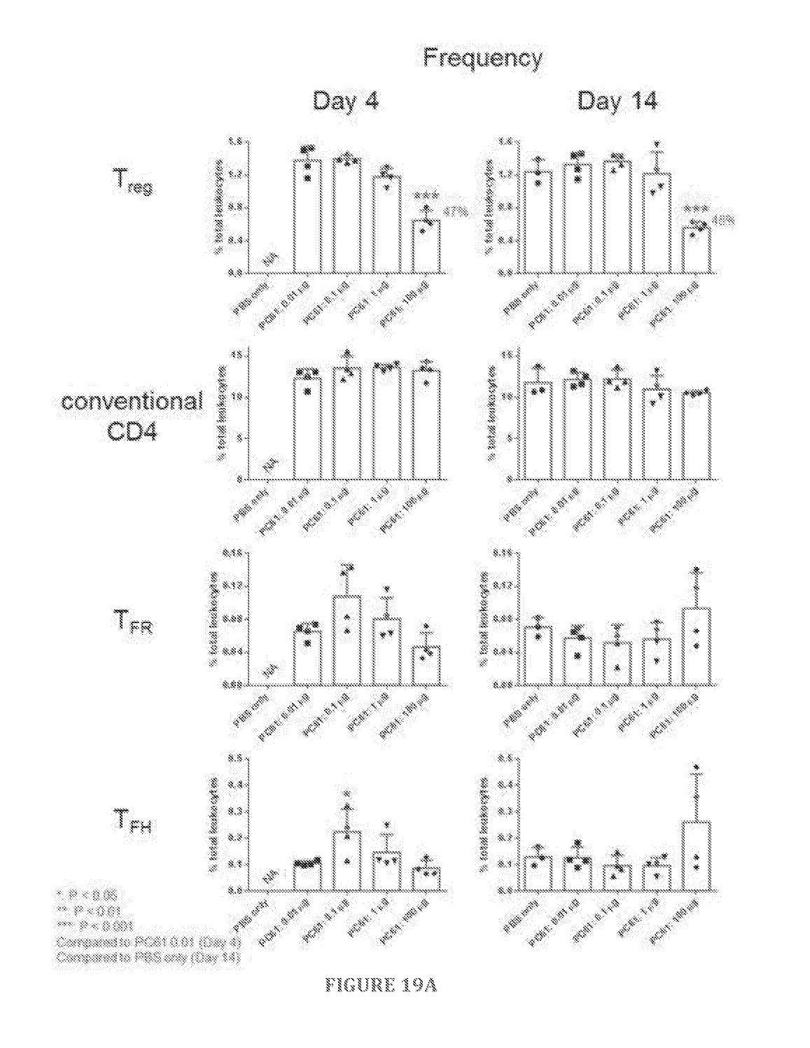

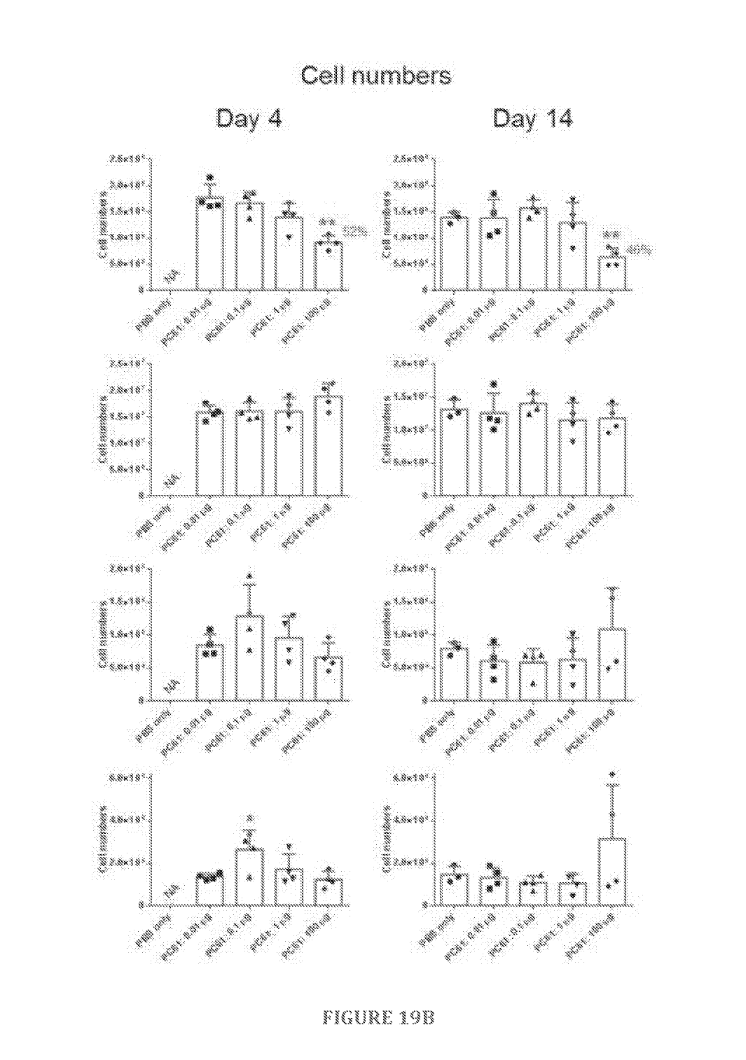

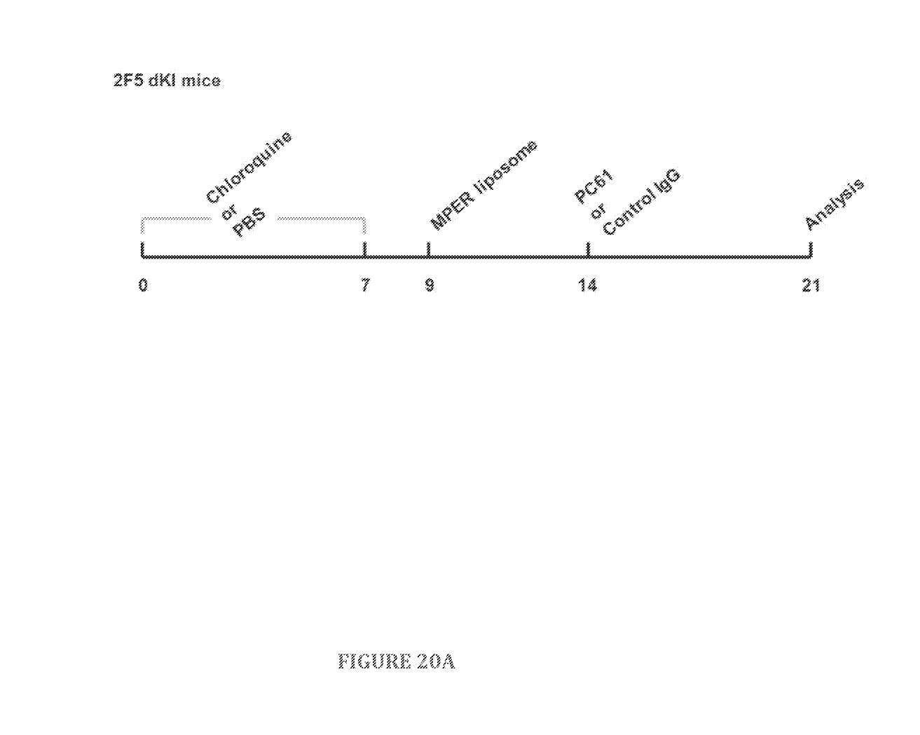

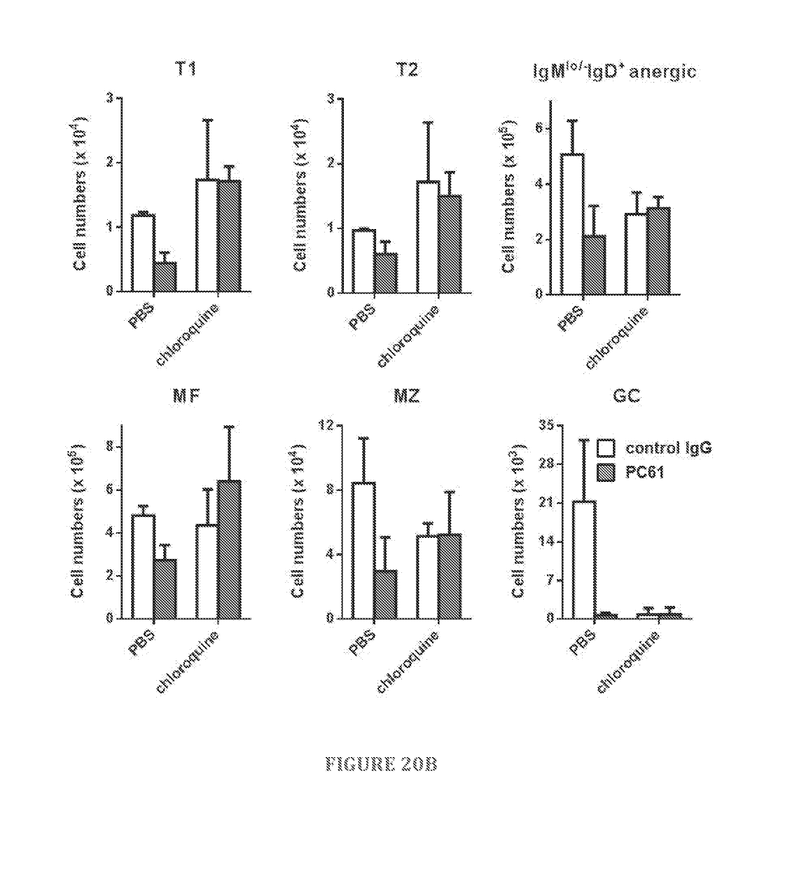

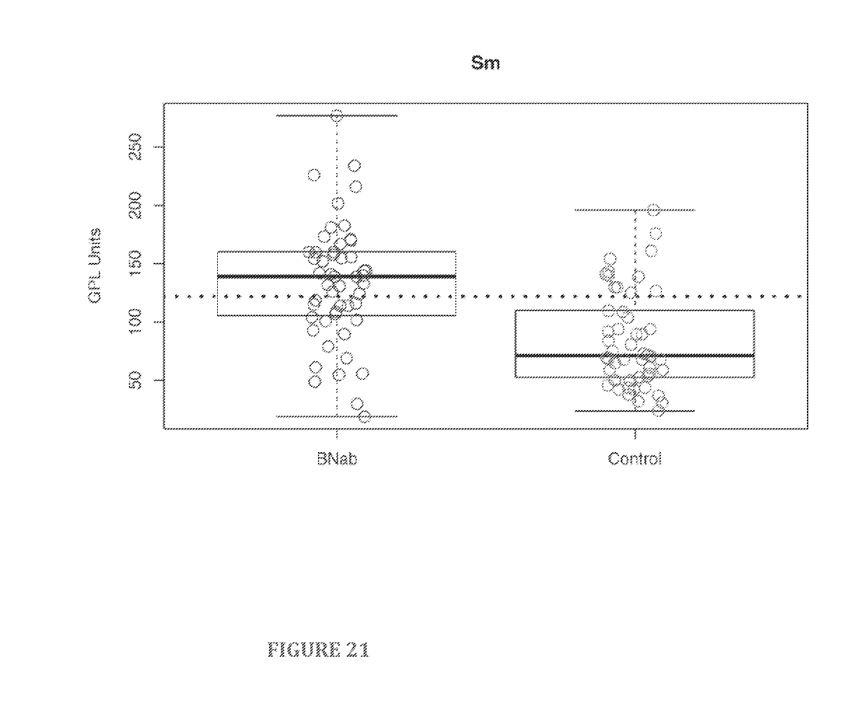

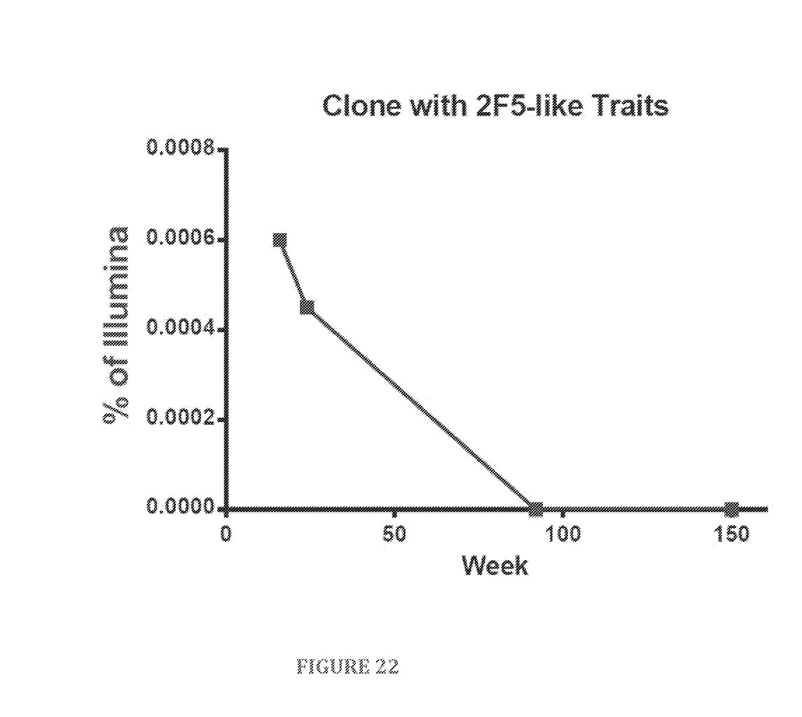

Of those chronically infected with HIV, it was investigated whether those who make bnAbs are more likely to have autoantibodies than those individual who do not. In one study of HIV infected individual there were 50 bnAbs and 51 no-Nabs individuals. Nine autoantibody tests were done: clinical assay AtheNA® luminex bead assay (RNP, SSA, SSB, SCL-70, Smith [Sm], double stranded DNA, histones, Jo-1, centromere B), and Cardiolipin ELISA. In this study 80% of 51 BnAbs individuals versus 38% of 50 non-BnAbs individuals had one or more autoantibody tests positive (Chi-square=18.8; p<0.0001). FIG. 21 shows that more HIV-1 chronics with BnAbs have positive assay for the Sm autoantigen compared to chronics with no BnAbs. Other studies by Illumina MiSeq Monitoring have shown that a macaque clonal lineage of 2F5 VH Genes with all key traits of the human 2F5 BnAb disappeared over time (FIG. 22).

There are various hypotheses why bnAbs are not routinely made. Studies on BnAb Biology and Host Control of BnAbs have shown that all bnAbs are unusual, BnAb traits predispose them to be deleted, edited, anergized or affinity reverted/redeemed away from autoreactivity. The result is that the pool of bnAb precursors is smaller than for the pool of non-bnAb precursors, i.e. bnAbs are subdominant. Subdominance could be due to smaller pool size and active host tolerance controls. Other studies have shown that bnAbs can be induced in both 2F5 and 4E10 knock-in mice by MPER-peptide liposome immunogens. While some BnAb B cells may be present these in lower numbers (due to tolerance deletion) with remaining cells in decreased activation state (anergy) (2F5, 4E10).

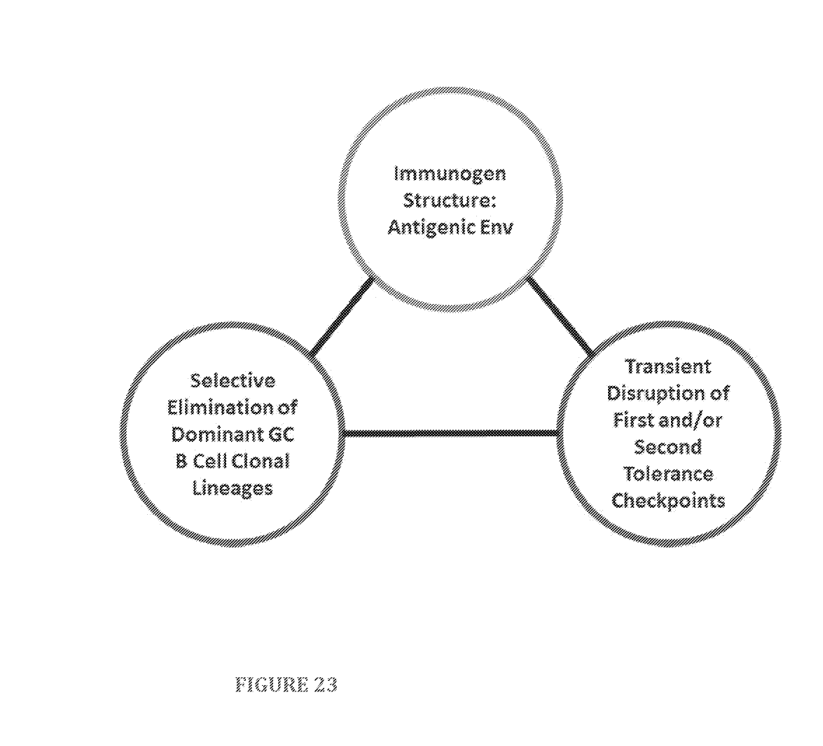

The implication of the above observations is that immunization with Env immunogens alone may not induce bnAbs to become dominant. The hypothesis is that bnAbs can be induced when there is transient modification to the host to break tolerance in the setting of vaccination. Transient modification of host tolerance mechanisms may be required to recreate the immunological milieu (FIG. 23). Some of the tolerance control mechanisms which might affect tolerance control of bnAbs are: PD-1 blockade; T reg depletion; CD40L hyperstimulation; Chloroquine administration; Soluble antigen administration. The programmed death 1 (PD-1) pathway is a negative feedback system that represses Th1 cytotoxic immune responses and that, if unregulated, can damage the host (see Lee et al. N Engl J Med 2015; 372:2509-2520Jun. 25, 2015) PD-1 expressed on TFH and expected to deliver negative signals to prevent uncontrolled TFH expansion and activity. See Crotty, S Ann. Rev. Immunol. 29: 621, 2011. PD-1 KO mice have decreased B cell function. It is possible that transient PD-1 blockade after vaccination have a salutary effect by releaving TFH inhibitory signals.

T reg depletion before cancer vaccine immunization induces long-lived anti-tumor T cell response (J. Immunol. 171: 5931-5939, 2003; Cancer Gene Therapy 14: 201-210, 2007). T reg depletion induces durable T cell responses to a malaria subdominant epitope (J. Immunol. 175: 7264-7273, 2005). It is possible to use Ab to IL-2Ra—anti-CD25 antibody to transiently modulate immune response in vaccination.

HIV envelope Gp120 induces T regs when administered without adjuvant and protects from graft vs. host disease in mice (Blood 114: 1263, 2009). CD4 ligation has proved immunosuppressive and tolerance induction in mice and NHPs. Clinical trials planned with anti-CD4 abs (Frontiers in Immunology 3: Jun. 18, 2012).

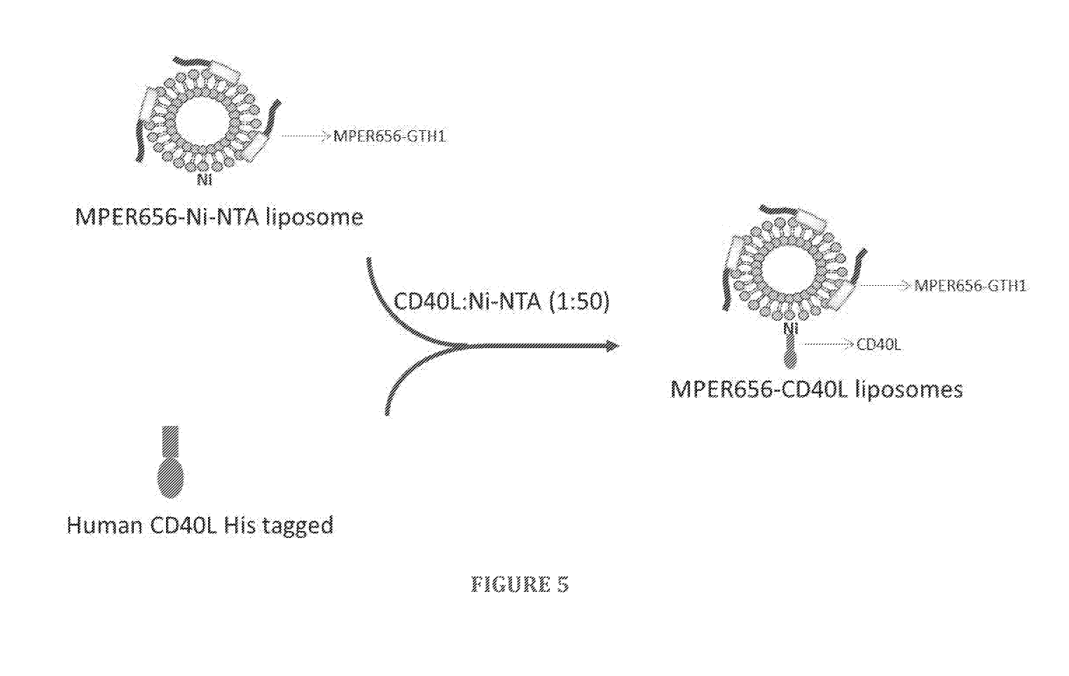

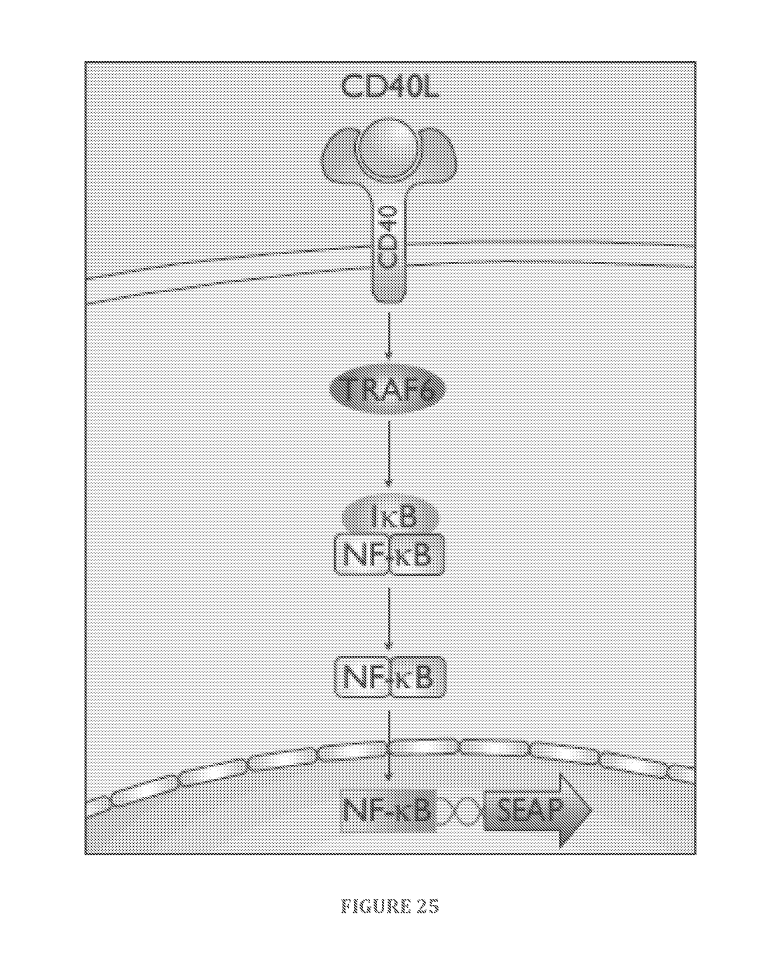

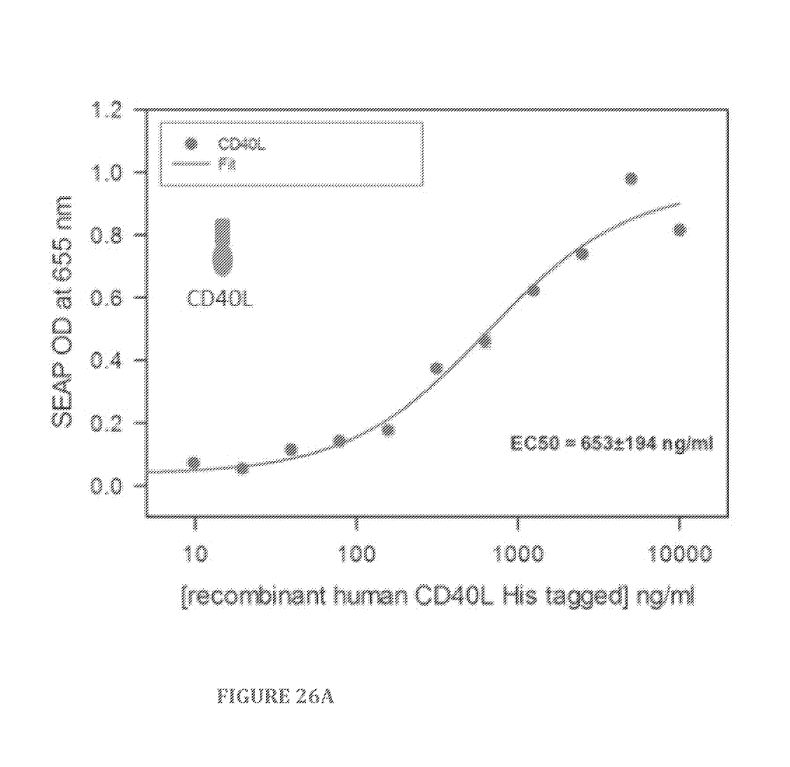

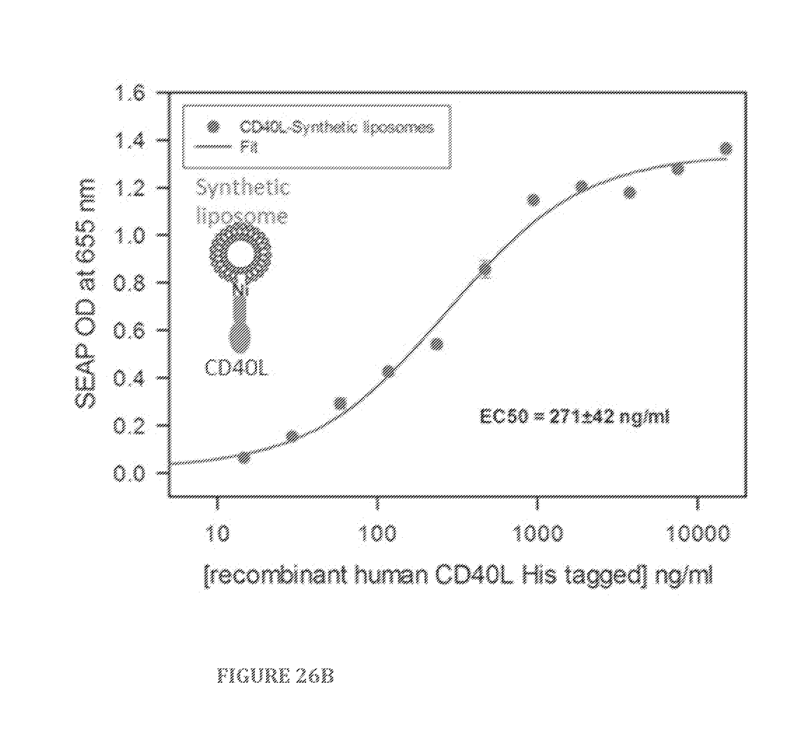

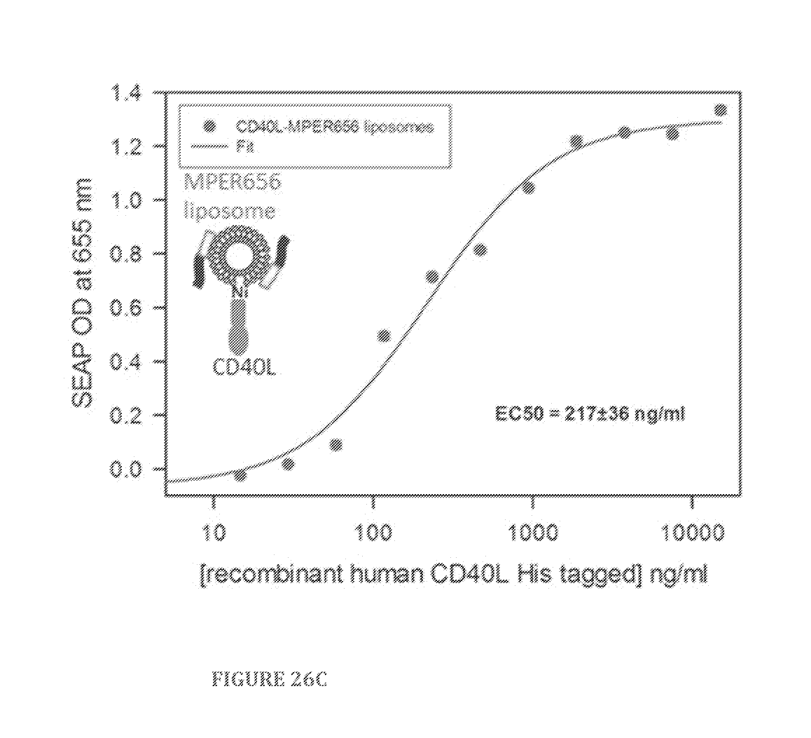

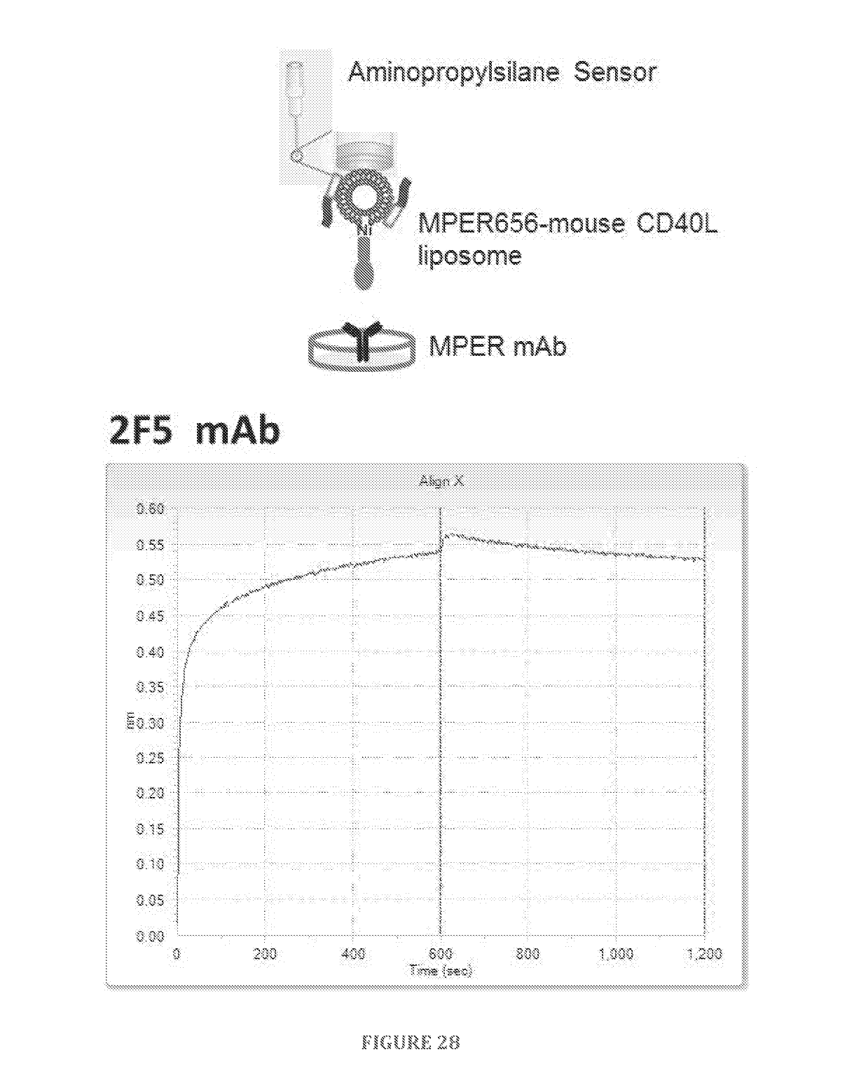

CD40 is a costimulatory protein found on antigen presenting cells and is required for their activation. The binding of CD154 (CD40L) on T follicular helper cells to CD40 activates antigen presenting cells and drives B cell activation. Excess CD40L (CD40L transgenic mice) rescued anti-Sm/RNP producing marginal zone B cells from apoptosis and led to plasma autoantibodies (PNAS 109: 7811-7816, 2012). In some embodiments the invention provides immunogen-liposome complexes with CD40Ligand (FIG. 5). FIGS. 26 and 28 show that conjugation of CD40L to liposomes enhances CD40 triggering.

In certain aspects the invention provides a strategy for induction of bnAbs which is to select and develop immunogens designed to recreate the antigenic evolution of Envs that occur when bnAbs do develop in the context of infection.

That broadly neutralizing antibodies (bnAbs) occur in nearly all sera from chronically infected HIV-1 subjects suggests anyone can develop some bnAb response if exposed to immunogens via vaccination. Working back from mature bnAbs through intermediates enabled understanding their development from the unmutated ancestor, and showed that antigenic diversity preceded the development of population breadth. See Liao et al. (2013) Nature 496, 469-476. In this study, an individual “CH505” was followed from HIV-1 transmission to development of broadly neutralizing antibodies. This individual developed antibodies targeted to CD4 binding site on gp120. In this individual the virus was sequenced over time, and broadly neutralizing antibody clonal lineage (“CH103”) was isolated by antigen-specific B cell sorts, memory B cell culture, and amplified by VH/VL next generation pyrosequencing. See Liao et al. (2013) Nature 496, 469-476.

Further analysis of envelopes and antibodies from the CH505 individual indicated that a non-CH103 Lineage participates in driving CH103-BnAb induction. For example V1 loop, V5 loop and CD4 binding site loop mutations escape from CH103 and are driven by CH103 lineage. Loop D mutations enhanced neutralization by CH103 lineage and are driven by another lineage. Transmitted/founder Env, or another early envelope for example W004.03, and/or W004.26, triggers naïve B cell with CH103 Unmutated Common Ancestor (UCA) which develop in to intermediate antibodies. Transmitted/founder Env, or another early envelope for example W004.03, and/or W004.26, also triggers non-CH103 autologous neutralizing Abs that drive loop D mutations in Env that have enhanced binding to intermediate and mature CH103 antibodies and drive remainder of the lineage (See Gao F et al. Cell 158: 481-91, 2014).

Recently, a new paradigm for design of strategies for induction of broadly neutralizing antibodies was introduced, that of B cell lineage immunogen design (Nature Biotech. 30: 423, 2012) in which the induction of bnAb lineages is recreated. It was recently demonstrated the power of mapping the co-evolution of bnAbs and founder virus for elucidating the Env evolution pathways that lead to bnAb induction (Nature 496: 469, 2013). From this type of work has come the hypothesis that bnAb induction will require a selection of antigens to recreate the “swarms” of sequentially evolved viruses that occur in the setting of bnAb generation in vivo in HIV infection (Nature 496: 469, 2013).

A critical question is why the CH505 immunogens are better than other immunogens. This rationale comes from three recent observations. First, a series of immunizations of single putatively “optimized” or “native” trimers when used as an immunogen have not induced bnAbs as single immunogens. Second, in all the chronically infected individuals who do develop bnAbs, they develop bnAbs in plasma after ˜2 years. When these individuals have been studied at the time soon after transmission, they do not make bnAbs immediately. Third, now that individual's virus and bnAb co-evolution has been mapped from the time of transmission to the development of bnAbs, the identification of the specific Envs that lead to bnAb development have been identified-thus taking the guess work out of env choice.

Two other considerations are important. The first is that for the CH103 bnAb CD4 binding site lineage, the VH4-59 and Vλ3-1 genes are common as are the VDJ, VJ recombinations of the lineage (Liao, Nature 496: 469, 2013). In addition, the bnAb sites are so unusual, the same VH and VL usage is recurring in multiple individuals. Thus, it can be expected that the CH505 Envs induce CD4 binding site antibodies in many different individuals.

Regarding the choice of gp120 vs. gp160, for the genetic immunization gp160 would normally not even be considered for use. However, in acute infection, gp41 non-neutralizing antibodies are dominant and overwhelm gp120 responses (Tomaras, G et al. J. Virol. 82: 12449, 2008; Liao, H X et al. JEM 208: 2237, 2011). Recently it was found that the HVTN 505 DNA prime, rAd5 vaccine trial that utilized gp140 as an immunogen, also had the dominant response of non-neutralizing gp41 antibodies. Thus, the use of gp160 vs gp120 for gp41 dominance will be evaluated early on.

The invention provides various methods to choose a subset of viral variants, including but not limited to envelopes, to investigate the role of antigenic diversity in serial samples. In other aspects, the invention provides compositions comprising viral variants, for example but not limited to envelopes, selected based on various criteria as described herein to be used as immunogens.

In other aspects, the invention provides immunization strategies using the selections of immunogens to induce cross-reactive neutralizing antibodies. In certain aspects, the immunization strategies as described herein are referred to as “swarm” immunizations to reflect that multiple envelopes are used to induce immune responses. The multiple envelopes in a swarm could be combined in various immunization protocols of priming and boosting.

Sequences/Clones

Described herein are nucleic and amino acids sequences of HIV-1 envelopes. In certain embodiments, the described HIV-1 envelope sequences are gp160s. In certain embodiments, the described HIV-1 envelope sequences are gp120s. Other sequences, for example but not limited to gp140s, both cleaved and uncleaved, gp140 Envs with the deletion of the cleavage (C) site, fusion (F) and immunodominant (I) region in gp4—named as gp140ΔCFI, gp140 Envs with the deletion of only the cleavage (C) site and fusion (F) domain—named as gp140ΔCF, gp140 Envs with the deletion of only the cleavage (C)—named gp140AC (See e.g. Liao et al. Virology 2006, 353, 268-282), gp145s, gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted: ((SEQ ID NO: 8)

MRVMGIQRNYPQWWIWSMLGFWMLMICNGMWVTVYYGVPVWKEAKTTLFC
ASDAKAYEKEVHNVWATHACVPTDPNPQE

(rest of envelope sequence is indicated as “ . . . ”). In other embodiments, the delta N-design described for CH505 T/F envelope can be used to make delta N-designs of other CH505 envelopes. CH505 Envelopes with delta N-terminal design are referred to as D8 or ΔN8 or deltaN8. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 11, amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain embodiments, the invention provides envelope sequences, amino acid sequences and the corresponding nucleic acids, and in which the V3 loop is substituted with the following V3 loop sequence TRPNNNTRKSIRIGPGQTFY ATGDIIGNIRQAH (SEQ ID NO: 9). This substitution of the V3 loop reduced product cleavage and improves protein yield during recombinant protein production in CHO cells.

In certain embodiments, the CH505 envelopes will have added certain amino acids to enhance binding of various broad neutralizing antibodies. Such modifications could include but not limited to, mutations at W680G or modification of glycan sites for enhanced neutralization.

In certain aspects, the invention provides composition and methods which use a selection of sequential CH505 Envs, as gp120s, gp 140s cleaved and uncleaved and gp160s, as proteins, DNAs, RNAs, or any combination thereof, administered as primes and boosts to elicit immune response. Sequential CH505 Envs as proteins would be co-administered with nucleic acid vectors containing Envs to amplify antibody induction.

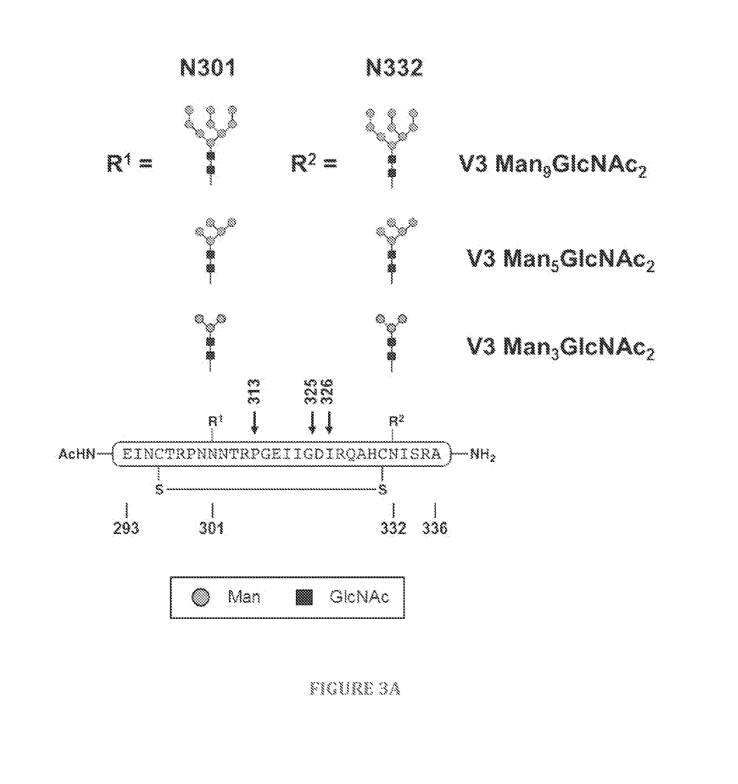

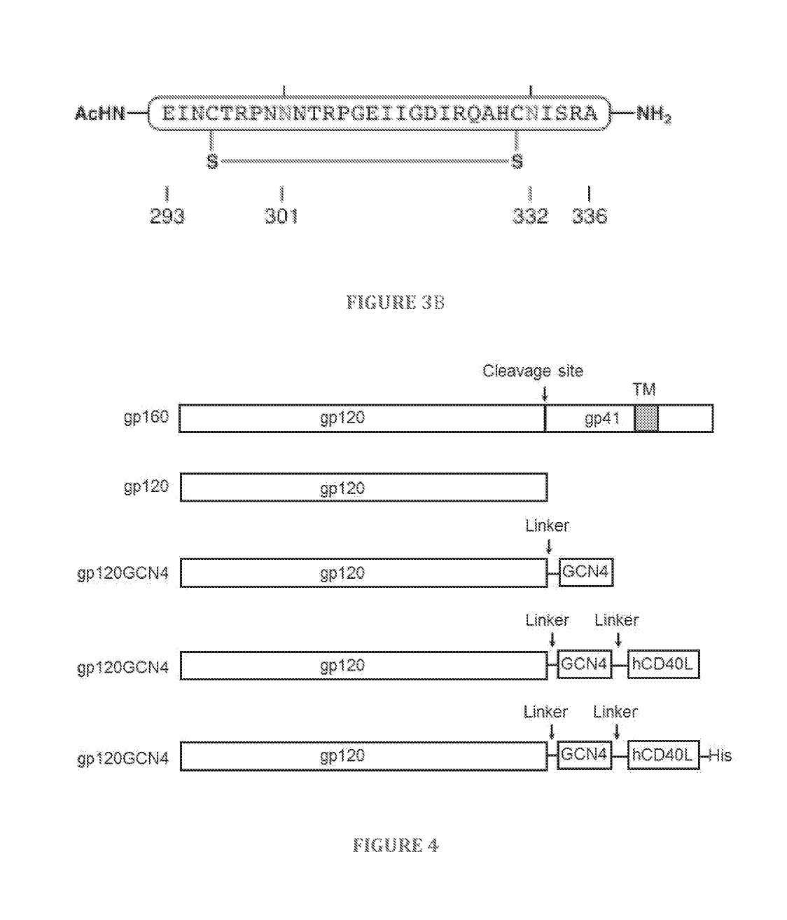

In certain embodiments the invention provides immunogens and compositions which include immunogens as trimers. In certain embodiments, the immunogens include a trimerization domain which is not derived from the HIV-1 envelope. In certain embodiments, the trimerization domain is GCN4 (See FIG. 4). In another embodiments, the trimerization domain could be CD40L. In other embodiments, the immunogens include CD40L domain (See FIGS. 4 and 5).

HIV-1 gp120 Trimer Vaccine Immunogens

HIV-1 Env Gp120 GCN4 Trimer

HIV-1 Env gp120 GCN4 trimer is designed as a fusion protein to be expressed as soluble recombinant trimeric HIV-1 gp120 protein. HIV-1 Env gp120 is mutated from residue R to E at the cleavage site of HIV-1 gp120 at the residue positions R503 and R511 (or any mutations at this region) to destroyed the cleavage site, a 6-residue linker (GSGSGS) (the linker can be variations of 3-20 residues in length) is added to the C-terminal end of HIV-1 gp120 followed by addition of 33 amino acid residues of GCN4 sequence (RMKQIEDKIEEILSKIYHIENEIARIKKLIGER (SEQ ID NO: 10)). For additional linkers see U.S. Pat. No. 8,597,658 incorporated by reference.

HIV-1 Env Gp120 GCN4 CD40L Trimer:

In certain embodiments the trimer design includes an immune co-stimulator

HIV-1 Env gp120 GCN4 CD40L trimer is designed as a fusion protein to be expressed as soluble recombinant trimeric HIV-1 gp120 protein co-expressed with functional CD40L as immune co-stimulator. HIV-1 Env gp120 is mutated from residue R to E at the cleavage site of HIV-1 gp120 at the residue positions R503 and R511 (or any mutations at this region) to destroyed the cleavage site, a 6-residue linker (GSGSGS (SEQ ID NO: 11)) (the linker can be variations of 3-20 residues in length) is added to the C-terminal end of HIV-1 gp120, 33 amino acid residues of GCN4 sequence (RMKQIEDKIEEILSKIYHIENEIARIKKLIGER (SEQ ID NO: 10)) is added to the C terminal end of the 6-residue linker, then a 11-residue liner (GGSGGSGGSGG (SEQ ID NO: 12)) (the linker can be variations of 3-20, 1, 2, 3, 4, 5, 6, 7, 8, 9, 10, 11, 12, 13, 14, 15, 16, 17, 18, 19, 20 residues in length) is added to the C terminal end of the GCN4 domain, followed by addition of the sequence of the functional extracellular domain of the human CD40 ligand (L) E113-L261.

HIV-1 Env Gp120 GCN4 CD40L Trimer with his Tag:

HIV-1 Env gp120 GCN4 CD40L trimer with His tag is designed as a fusion protein to be expressed as soluble recombinant trimeric HIV-1 gp120 protein co-expressed with functional CD40L as immune co-stimulator. HIV-1 Env gp120 is mutated from residue R to E at the cleavage site of HIV-1 gp120 at the residue positions R503 and R511 (or any mutations at this region) to destroyed the cleavage site, a 6-residue linker (GSGSGS) (the linker can be variations of 3-20 residues in length) is added to the C-terminal end of HIV-1 gp120, 33 amino acid residues of GCN4 sequence (RMKQIEDKIEEILSKIYHIENEIARIKKLIGER (SEQ ID NO: 10)) is added to the C terminal end of the 6-residue linker, a 11-residue liner (GGSGGSGGSGG (SEQ ID NO: 12)) (the linker can be variations of 3-20 residues in length) is added to the C terminal end of the GCN4 domain, then the sequence of the functional extracellular domain of the human CD40 ligand (L) E113-L261 is then added followed by addition of 10 histidine residues as tag (the His tag can be more or less of 10 residues). His-tag is added to anchor the HIV-1 gp120GCN4 CD40L into liposome through nickel.

Using the instant disclosure of envelope timers, any HIV-1 envelope can be designed as a trimer. In certain embodiments the trimer designs can include any suitable linker, for example but not limited to linkers described in U.S. Pat. No. 8,597,658.

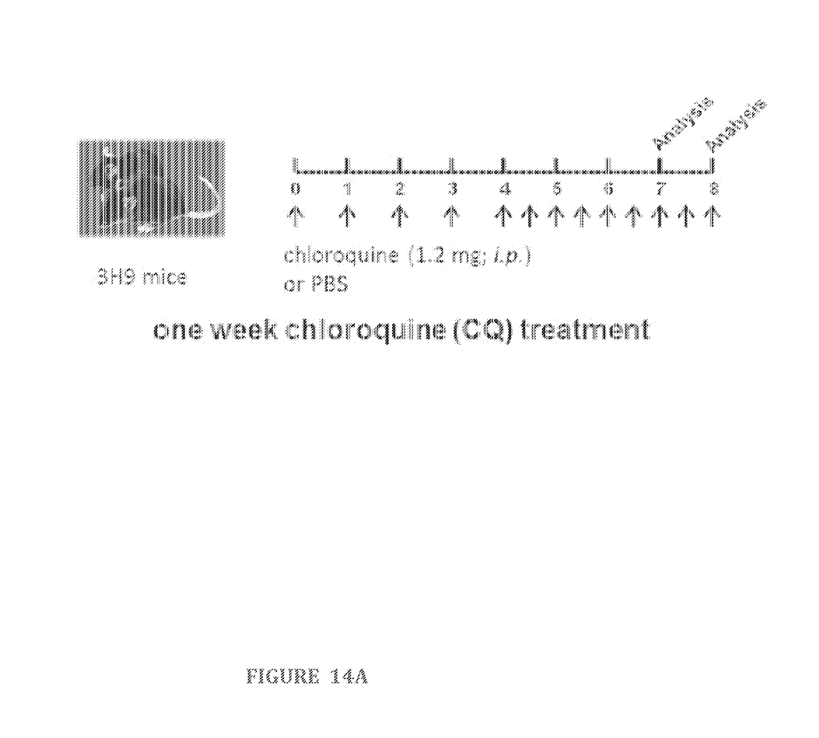

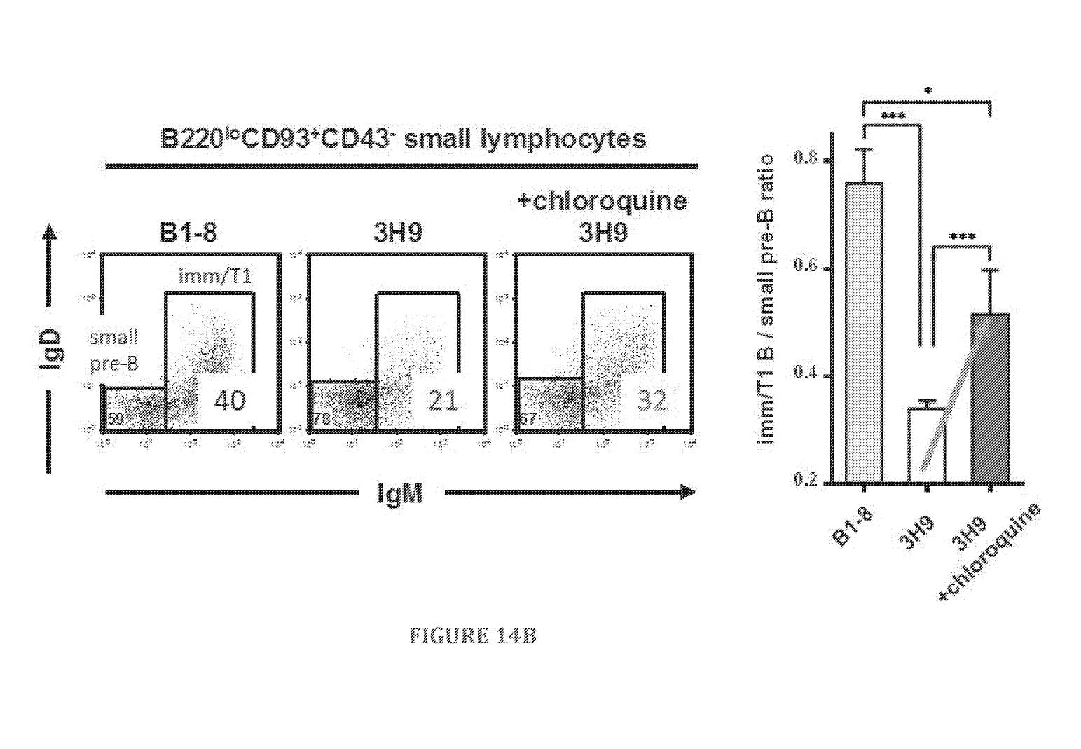

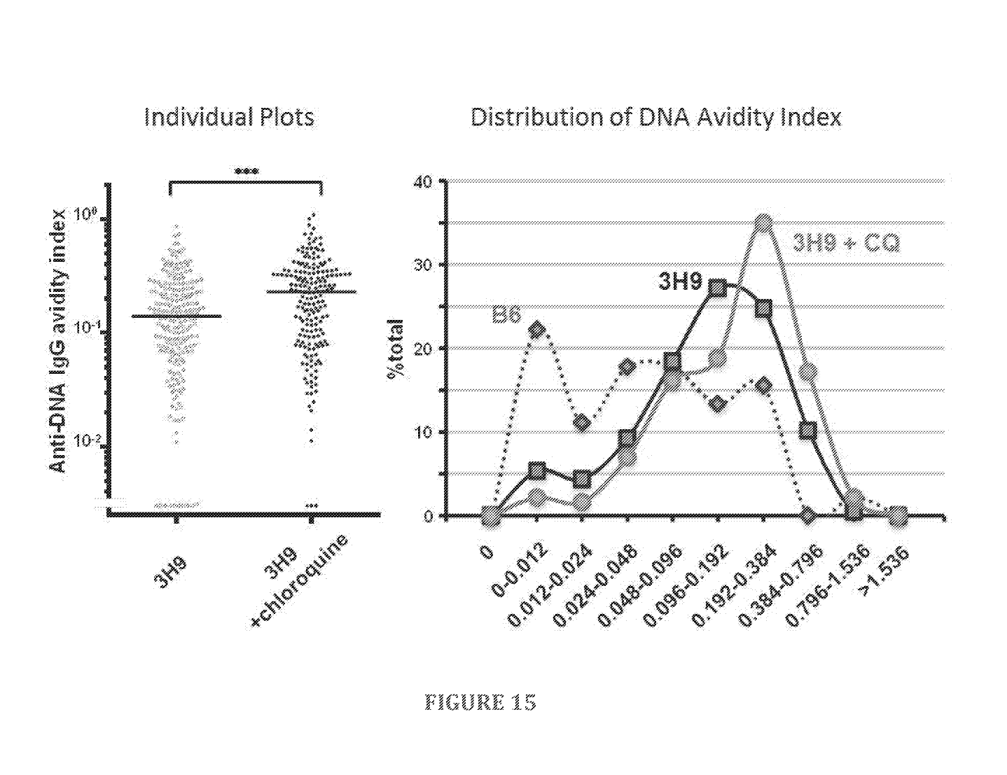

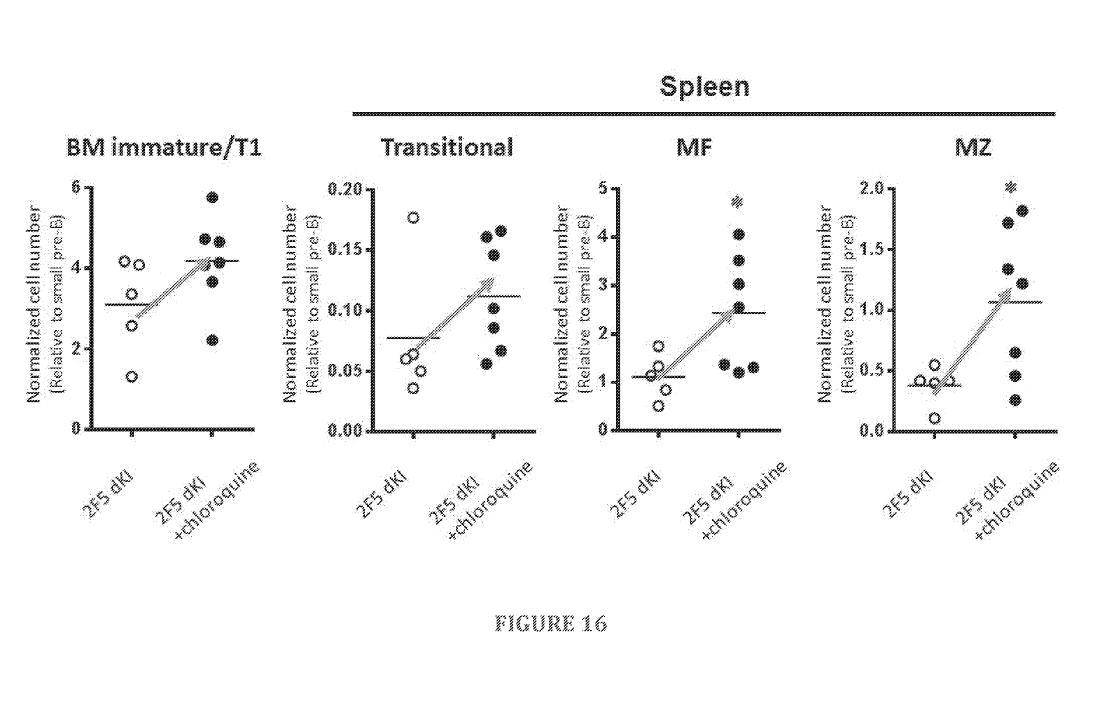

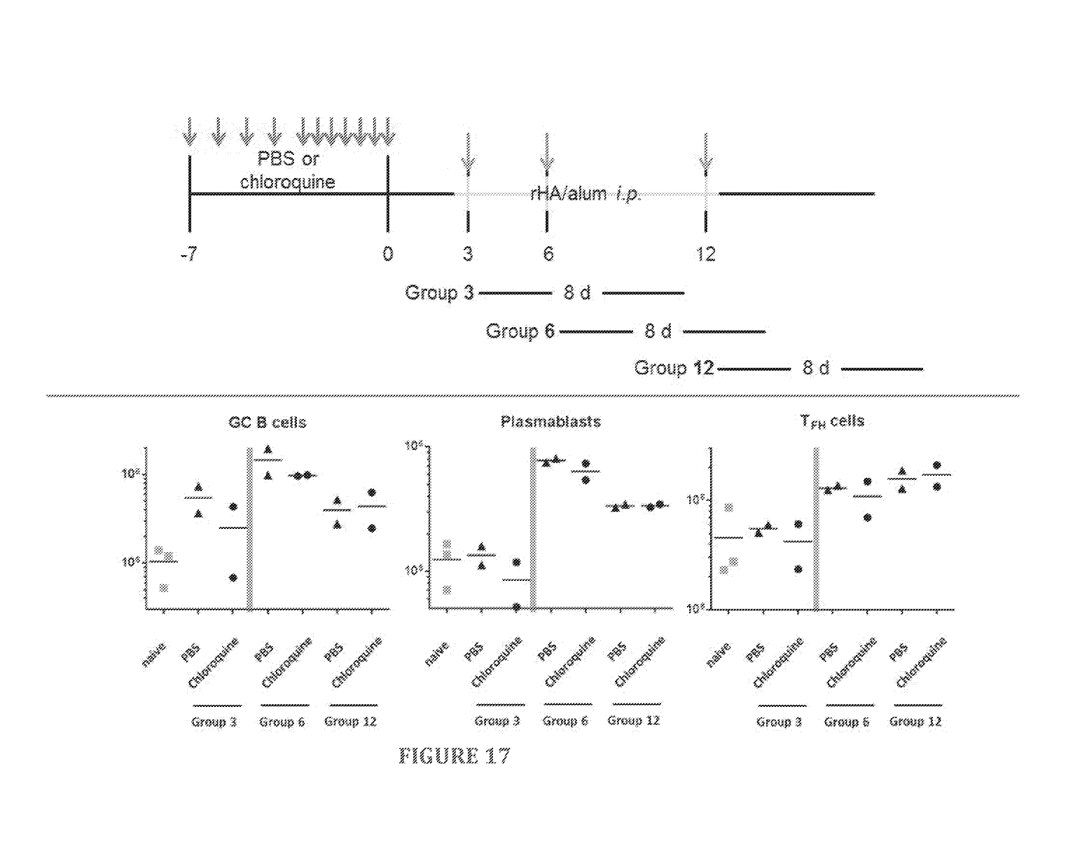

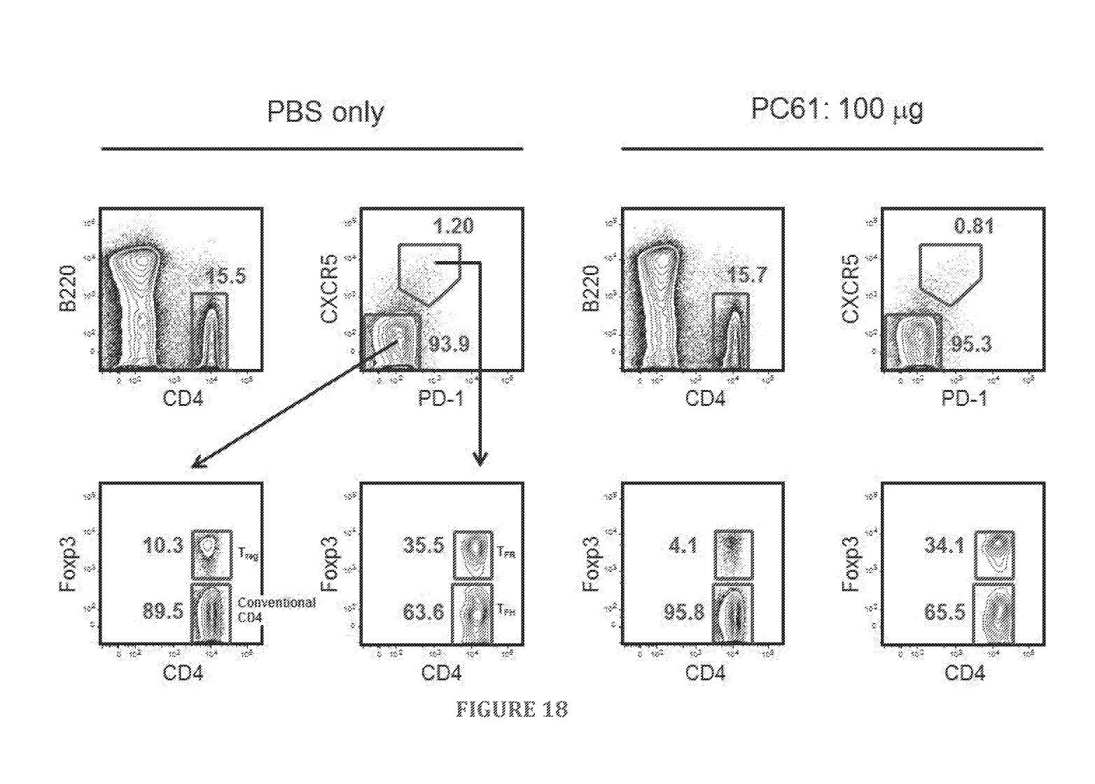

In certain embodiments the HIV-1 immunogen contemplated for use in the invention is any immunogen capable of inducing bnAbs against HIV-1 envelopes and epitopes therein. In non-limiting embodiments the immunogen is any one of the HIV-1 envelopes or selection of envelopes in Application WO2014042669 (PCT/US PCT/US2013/000210), U.S. Application Ser. No. 61/955,402 (“Swarm Immunization with Envelopes form CH505” Examples 2-4, FIGS. 14-19); U.S. Application Ser. Nos. 61/972,531 and 62/027,427 (Examples 2-3, FIGS. 18-24) the contents of which applications are herein incorporated by reference in their entirety.

In non-limiting embodiments the immunogen is selected from the following:

CH505 Envs to induce CD4 bs Abs. See WO2014042669; U.S. Application Ser. No. 61/955,402 (“Swarm Immunization with Envelopes form CH505” Examples 2-4, FIGS. 14-19); US Application Ser. Nos. 61/972,531 and 62/027,427.

CH848 Envs to induce V3 glycan Abs. See U.S. Application Ser. No. 61/972,649.

VRC26 Envs to induce V1V2 glycan Abs. Doria-rose N A et al. Nature 509: 55-62, 2014

CH01-CH04 bnAb heterologous Envs-V1V2 glycan Liao H X et al. Immunity 38: 176, 2013; Bonsignori M et al J. Virol 85: 9998, 2011

CH1754 Envs to induce CD4 bs Abs. See U.S. Application Ser. No. 61/884,014.

Group M B cell Mosaic Envs. See U.S. Patent Application Ser. No. 61/884,696

Series of CAP206 Envs to induce MPER Abs.

MPER peptide liposomes to induce MPER Abs. Non-limiting examples of MPER peptides are MPER656 of sequence NEQELLELDKWASLWNWFNITNWLWYIK (SEQ ID NO: 1); MPER656.1 of sequence NEQDLLALDKWASLWNWFDISNWLWYIK (SEQ ID NO: 2); MPER656.2 of sequence NEKDLLALDSWKNLWNWFSITKWLWYIK (SEQ ID NO: 3); MPER656.3 of sequence NEQELLALDKWNNLWSWFDITNWLWYIR (SEQ ID NO: 4); CAP206_0moB5_MPER656 of sequence NEKDLLALDSWKNLWNWFDITKWLWYIK (SEQ ID NO: 13). In certain embodiments, these peptide include an anchor/linker at the C-terminal end. The linker could be GTH1 (YKRWIILGLNKIVRMYS (SEQ ID NO: 9)). See US Pub 20110159037; U.S. Serial Application No. 61/883,306. See Verkoczy L et al. J. Immunol 191: 2538, 2013; Dennison S M et al. PLOS One 6: e27824, 2011).

Membrane-bound trimers—CH505, JRFL. See U.S. Application Ser. No. 61/941,902, and U.S. Application Ser. No. 61/973,414.

A244 gp120 to induce glycan and V1V2 bnAbs. See Alam S M J. viriology 87: 1554, 2013.

V1V2 Peptide-Glycans to induce V1V2 glycan bnAbs. See WO2014066889; Alam S M et al PNAS USA 110: 18214, 2013.

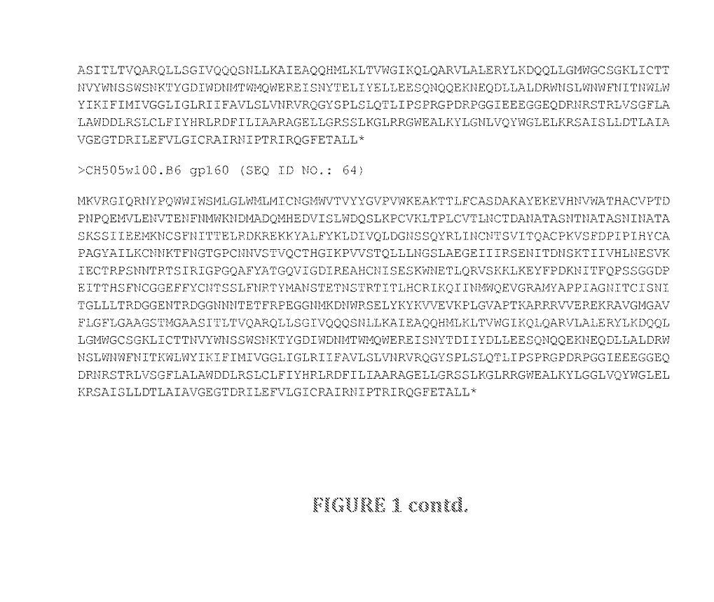

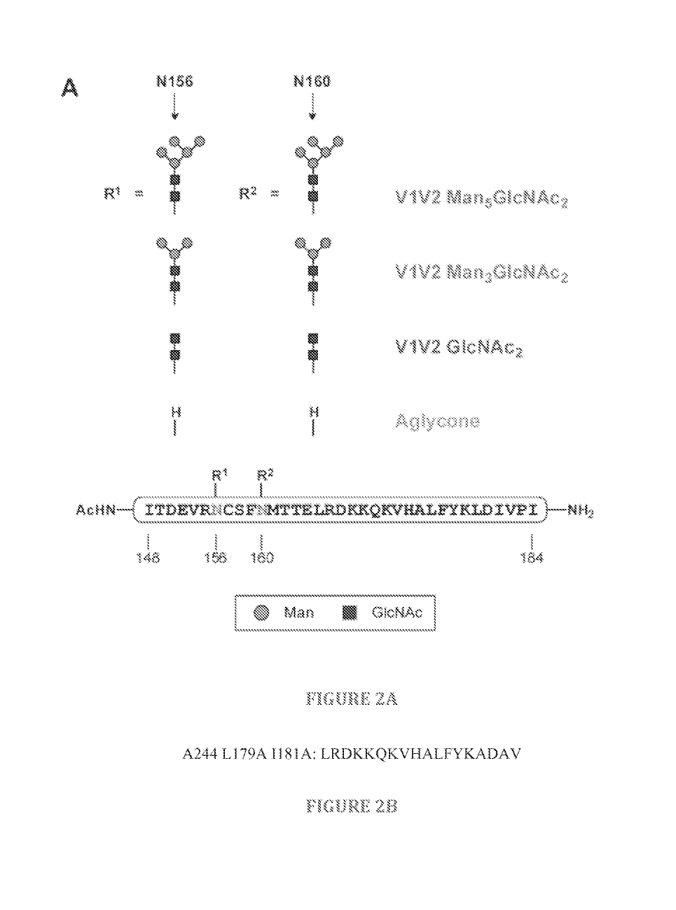

V1V2 tags recombinant protein-V1V2 glycan bnAbs. See FIG. 2; Liao et al. Immunity 38: 176, 2013.

V3 Peptide-Glycans to induce V3 glycan bnAbs. See FIG. 3; See PCT/US2014/034189.

Any other founder Envs that are antigenic for bnAb epitopes. Liao H X et al J. Virology 87: 4185, 2013

In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (Barouch D H, et al. Nature Med. 16: 319-23, 2010), recombinant mycobacteria (i.e., rBCG or M smegmatis) (Yu, J S et al. Clinical Vaccine Immunol. 14: 886-093, 2007; ibid 13: 1204-11, 2006), and recombinant vaccinia type of vectors (Santa S. Nature Med. 16: 324-8, 2010), for example but not limited to ALVAC, replicating (Kibler K V et al., PLoS One 6: e25674, 2011 nov 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See www.incellart.com/en/research-and-development/technologies.html.

In certain aspects the invention contemplates using immunogenic compositions wherein immunogens are delivered as recombinant proteins. Various methods for production and purification of recombinant proteins suitable for use in immunization are known in the art.

The immunogenic envelopes can also be administered as a protein boost in combination with a variety of nucleic acid envelope primes (e.g., HIV −1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, TLR agonists are used as adjuvants. In some embodiments, the TLR agonist is a TLR4 agonist, such as but not limited to GLA/SE. In other embodiment, adjuvants which break immune tolerance are included in the immunogenic compositions. In some embodiments the adjuvant is TLR7 or a TLR7/8 agonist, or a TLR-9 agonist, or a combination thereof. See PCT/US2013/029164.

Host Mechanisms Control bNAbs and Transient Immunomodulation During Vaccination

There are various host mechanisms that control bNAbs. For example highly somatically mutated antibodies become autoreactive and/or less fit (Immunity 8: 751, 1998; PloS Comp. Biol. 6 e1000800, 2010; J. Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER BnAb induction (deletion, anergy, receptor editing). See J Immunol 187:3785, 2011; J Immunol 191:1260, 2013; J Immunol 191:3186, 2013. Other variations of tolerance control likely will be operative in limiting BnAbs with long HCDR3s, high levels of somatic hypermutations. 2F5 and 4E10 BnAbs were induced in mature antibody knock-in mouse models with MPER peptide-liposome-TLR immunogens. See J Immunol 191:2538, 2013; AIDS Res Hum Retrov 29:OA02.06, 2013.

All of the bnAbs identified so far have unusual characteristics and many are autoreactive. These bnAb traits predispose them to be deleted, edited, anergized or affinity reverted which likely prevents the induction of such bnAbs in vaccination settings.

In certain aspects the invention provides that to induce bnAbs by vaccination, there is a need to transiently modify the host to break tolerance mechanism—transient immunomodulation during vaccination. Breaking tolerance would overcome host controls and lead to induction and expansion of B cell clones of bnAbs. In certain embodiments the immunogens, methods and compositions of the invention comprise agents which modulate transiently host immune response mechanisms. In certain embodiments the immunogens, methods and compositions of the invention comprise immunomodulatory components. In a non-limiting embodiment, the immunogen is a fusion peptide which comprises CD40L.

Immunomodulatory Agents

In certain embodiments the invention provides agents, immunization methods and compositions which modulate the bone marrow/first tolerance checkpoint, agents which modulate the immune responses in the periphery, and agents which modulate B cell development at both checkpoints. Non-limiting examples of this modulation include PD-1 blockade; T regulatory cell depletion; CD40L hyperstimulation; soluble antigen administration, wherein the soluble antigen is designed such that the soluble agent eliminates B cells targeting dominant epitopes.

In non-limiting embodiments, these agents include PTP1B inhibitors, e.g. PTP1B Inhibitor—CAS 765317-72-4—Calbiochem (Wiesmann, C., et al. 2004. Nat. Struct. Mol. Biol. 11, 730), MSI 1436 (See Krishnan et al. Nature Chemical Biology 10, 558-566 (2014); chloroquine; clodronate or any other bisphosphonate; Foxo1 inhibitors, e.g. 344355|Foxo1 Inhibitor, —Calbiochem (See Nagashima et al. Mol Pharmacol 78:961-970, 2010).

Foxo1 is a key downstream target of the PI3K signaling cascade involved in shutting off RAG expression/promoting positive B cell selection, shutting it off should therefore release cells from central tolerance (See Amin and Schlissel NATURE IMMUNOLOGY VOLUME 9 NUMBER 6 Jun. 2008 pp. 613-622 and Chow et al refs). STI-571 (Gleevac) is used to inhibit the PTK that regulates foxo1 (See Amin and Schlissel in NATURE IMMUNOLOGY VOLUME 9 NUMBER 6 Jun. 2008 pp. 613-622. Although this appears critical in very early B-cells, it may be relevant for enriching for the initial bnAb repertoire, for example for long CDHR3 bnAbs that could be counterselected at the pre-ag stage. Foxo1 has multiple critical roles in B-cell development e.g. regulation of AID SHM (see Dengler et al 2008 NATURE IMMUNOLOGY VOLUME 9 NUMBER 12 Dec. 2008 pp. 1388-1398) and thus if its inhibition has a large effect in breaking central tolerance, may be useful not only in primes, could also be useful for modulating SHM levels in later boosts.

Other targets for immunomodulation as part of a vaccination schedule to elicit bNAbs are PI3K and downstream, negatively-regulated targets of PI3K, for example the delta isoform of PKC and GSK3a/b. See Verkoczy et al 2007 J Immunol 2007; 178:6332-6341 showing importance of the PI3K pathway in the first B cell tolerance checkpoint; See also Mecklenbrauker, I., Saijo, K., Zheng, N. Y., Leitges, M. and Tarakhovsky, A., Protein kinase Cdelta controls self-antigen-induced Bcell tolerance. Nature 2002. 416: 860-865; Miyamoto, A., Nakayama, K., Imaki, H., Hirose, S., Jiang, Y., Abe, M., Tsukiyama, T., Nagahama, H., Ohno, S., Hatakeyama, S. and Nakayama, K. I., Increased proliferation of B cells and autoimmunity in mice lacking protein kinase Cdelta. Nature 2002. 416: 865-869; Limnander, A., Depeille, P., Freedman, T. S., Liou, J., Leitges, M., Kurosaki, T., Roose, J. P. and Weiss, A., STIM1, PKC-delta and RasGRP set a threshold for proapoptotic Erk signaling during B cell development. Nat Immunol 2011. 12: 425-433, which show in the HEL model that PKDdelta is involved in peripheral tolerance/anergy.

Lithium chloride or carbonate are non-limiting examples of GSK inhibitors, and Rottlerin (3′-[(8-Cinnamoyl-5,7-dihydroxy-2,2-dimethyl-2H-1-benzopyran-6-yl)methyl]-2′,4′,6′-trihydroxy-5′-methylacetophenone) is a non-limiting example of PKCdelta-inhibitor.

In certain embodiments, these agents include as non-limiting examples anti-CD25 Abs to deplete Tregs. In other embodiments the agents include CCR4 inhibitors to modulate T cells—specifically effector human Tregs express CCR4, (but not naive T cells, Th1, and CTLs). See Bayry et al. Trends in Pharmacological Sciences, April 2014, Vol. 35, No. 4 163-165.

In certain embodiments the CCR4 inhibitor is ani-CCR4 antibody. In other embodiments, the CCR4 inhibitor is AF399/420/18 025 (Inserm U872) (see Pere et al. BLOOD, 3 Nov. 2011_VOLUME 118, NUMBER 18, p. 4853-4862), CCR4 inhibitor is CB20, CCR4 inhibitor is SP50, CCR4 inhibitor is CAS 864289-85-0 (Santa Cruz).

In certain embodiments, the method comprise administering an agent which modulates germinal center (GC) responses, in an amount sufficient to eliminate dominant B-cell clones, thereby providing an opportunity for a sub-dominant B cell clone expansion. See Han et al. J. Exp Med (1995), vol 182: 1635-1644. The agent is an immunogen/soluble antigen, for example but not limited to a peptide derived from HIV-1 envelope, is designed such that it binds to B-cells with receptors for dominant epitopes, but does not bind or binds less well to B cells with receptors for subdominant epitopes. The timing and amount of administering of this agent is critical, and in non-limiting embodiments this agent is administered shortly post vaccination with the immunogen of interest.

The immunostimulatory agents are administered at times appropriate for selection against unwanted GC B-cells. This selection is known to be active during the first and second thirds of the primary GC reaction; in primary immune responses, this period generally comprises days 5-12 post immunization. In some embodiments the agent is administered on day 5, 6, 7, 8, 9, 9, 11, 12, 13, or 14 after immunization to not interfere with T follicular helper cell induction of the GC but to rather interfere with T regulatory cell dampening of clones that are desired. The agent is administered in an amount which is in excess of an amount needed for triggering B cell responses. In experimental animal models, this dose (given i.v. and/or i.p.) has ranged from about 10 mg/kg to 0.30 mg/kg. Using this as guidance, a skilled artisan can readily determine the dose of soluble antigen that effective, including the minimum effective dose, to achieve selection against GC B cells.

For agents that interfere with the first tolerance checkpoint in bone marrow, for example but not limited to chloroquine or its analogues, these agents would be administered for several days, for example but not limited to 1, 2, 3, 4, 5, 6, 7, 8, 9, 10, 11, 12, 13 or 14 days before and up to 7-10, 1, 2, 3, 4, 5, 6, 7, 8, 9, or 10 days after each immunization. A skilled artisan can readily determine the time of immunization in conjunction with treatment of immunomodulatory agent of the first and/or second checkpoint.

Similar regiment could be used for agents that interfere with the second tolerance checkpoint (ie tolerance checkpoints in the periphery) such as CD25 antibody. One embodiment of the invention is to administer the anti-CD25 antibody in low doses (such as 1-5 mg or less) IV or IM approximately 5-7, 5-12, 5-15, 1, 2, 3, 4, 5, 6, 7, 8, 9, 10, 11, 12, 13, 14, 15 days after immunization to allow T help to occur and germinal center reactions to be started, and then to disrupt the T regulatory cell down-modulation of disfavored broadly neutralizing B cell lineages (bnAbs). For those bnAb lineages where both the first and second checkpoints are involved in limiting them (such as MPER gp41 bnAbs), it can be envisioned that combinations of first and second checkpoint inhibitors can be co-administered in such regimens as described above. For those bnAb lineages where just the second checkpoint tolerance mechanisms in the periphery are thought to be limiting such as for loop binding CD4 binding site antibodies, only those agents such as, but not limited to, CD25 antibodies would be administered as noted above.

In a non-limiting embodiment, a gp41 soluble immunogen is the agent which binds to B cells with dominant gp41 epitopes, but does not bind to B cells with subdominant epitopes within gp120. In certain embodiments, these B cells express receptors for subdominant epitopes for bnAbs, e.g. a CD4 bs. In a non-limiting embodiment, the gp41 soluble agent is administered in a vaccination method using non-gp120 HIV-1 envelope as an immunogen, e.g. gp140, gp145, gp160. In a non-limiting embodiment, aglycone V3 peptide is administered as the soluble antigen in an immunization regimen using V3 glycopeptide as an immunogen (See FIG. 3). In a non-limiting embodiment, V1V2 peptide is administered as the soluble antigen in an immunization regimen using V1V2 glycopeptide as an immunogen (See FIG. 2).

In certain embodiments, the agents may be administered prior to, with, or after the immunogen. Dosing could be readily determined by a skilled artisan, such that the immunomodulation is transient. Dosing range could be about 1.25 μM (˜1 μg/mL) for PTP1B inhibitor, 250 mg/mL for the bisphosphonates, and 0.43 μg/mL for the Foxo1 inhibitor AS1842856.

In certain embodiments, the invention provides formulations wherein these agents are formulated in a manner that permits coadministration with the immunogen such that the compound may be released in a controlled manner (e.g., via polylactate/polyglycolate particles) so that the immunomodulatory agent will be present during any or all of the phases of the immune response. Existing data on these agents (see supra for respective citations) show that their effects are not permanent, and so the biological effect on immune modulation will be transient in the absence of continued administration. These immunomodulatory agents may be used singularly or in combination. These agents may also be administered at a single time point or at multiple time points, and may be administered prior to, with, and/or following the priming immunization and/or the boosting immunization(s) as might be necessary for production of bnAb. The composition comprising the immunogens to induce immune responses might be administered multiple times (multiple boosts) after treatment with the immunomodulatory agents of the invention.

### EXAMPLES

### Example 1: GCN4 Envelope Trimers and CD40L Containing Immunogens Bind HIV-1 Envelope Antibodies and are Functionally Active

Provided is one example of the design and formulation of liposomes that present immune-modulating CD40 ligand (CD40L) and HIV-1 gp41 neutralizing antigen. CD40L, the ligand for CD40 expressed on B-cell surface is anchored on the liposomes that had HIV-1 gp41 MPER peptide immunogen conjugated in them. Two broadly neutralizing gp41 membrane proximal external region (MPER) antibodies (2F5, 4E10) bound strongly to CD40L conjugated MPER peptide liposomes. This construct has important application as an experimental AIDS vaccine in providing immune-modulating effect to stimulate proliferation of B-cells capable of producing neutralizing antibodies targeting HIV-1 gp41 MPER region. CD40L-gp41 MPER peptide-liposome conjugates: Recombinant CD40L with an

N-terminal Histidine Tag (MGSSHHHHHH SSGLVPRGSH
MQKGDQNPQI AAHVISEASS KTTSVLQWAE KGYYTMSNNL
VTLENGKQLT VKRQGLYYIY AQVTFCSNRE ASSQAPFIAS
LCLKSPGRFE RILLRAANTH SSAKPCGQQS IHLGGVFELQ
PGASVFVNVT DPSQVSHGTG FTSFGLLKL (SEQ ID NO: 14))

was anchored to MPER peptide liposomes via His-Ni-NTA chelation by mixing CD40L with MPER656-Ni-NTA liposomes at 1:50 CD40L and Ni-NTA molar ratio (Figure-5).

The construction of MPER peptide Ni-NTA liposomes utilized the method of co-solubilization of MPER peptide having a membrane anchoring amino acid sequence and synthetic lipids 1-Palmitoyl-2-Oleoyl-sn-Glycero-3-Phosphocholine (POPC), 1-Palmitoyl-2-Oleoyl-sn-Glycero-3-Phosphoethanolamine (POPE), 1,2-Dimyristoyl-sn-Glycero-3-Phosphate (DMPA), Cholesterol and 1,2-dioleoyl-sn-Glycero-3-[(N-(5-amino-1-carboxypentyl)iminodiacetic acid)succinyl] (nickel salt) (DGS-NTA(Ni) at mole fractions 0.216, 35.00, 25.00, 20.00, 1.33 and 10 respectively. Appropriate amount of MPER peptide dissolved in chloroform-methanol mixture (7:3 v/v), appropriate amounts of chloroform stocks of phospholipids were dried in a stream of nitrogen followed by over night vacuum drying. Liposomes were made from the dried peptide-lipid film in phosphate buffered saline (pH 7.4) using extrusion technology.

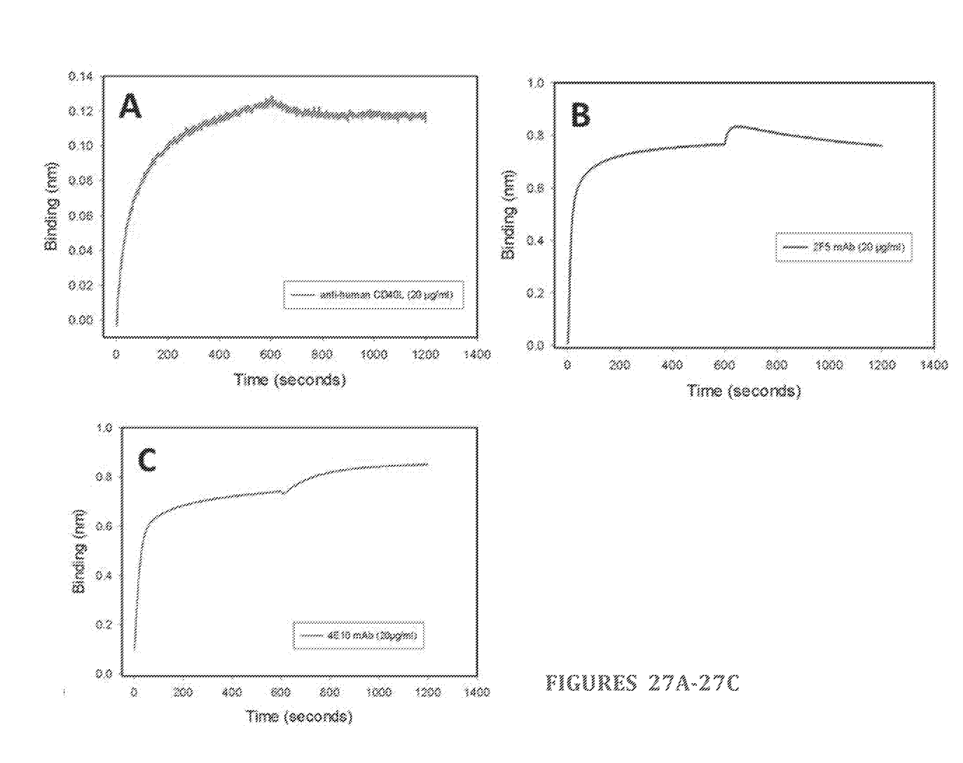

Biolayer interferometry (BLI) assay showed the binding of anti-human CD40L antibody to CD40L-MPER656 liposomes and confirmed the correct presentation of CD40 L on liposome surface (FIG. 5). The broadly neutralizing HIV-1 gp41 MPER antibodies 2F5 and 4E10 bound strongly to CD40L-MPER656 liposomes FIG. 27 and demonstrated that the CD40L co-display did not impede the presentation of the epitopes of 2F5 and 4E10 mAbs.

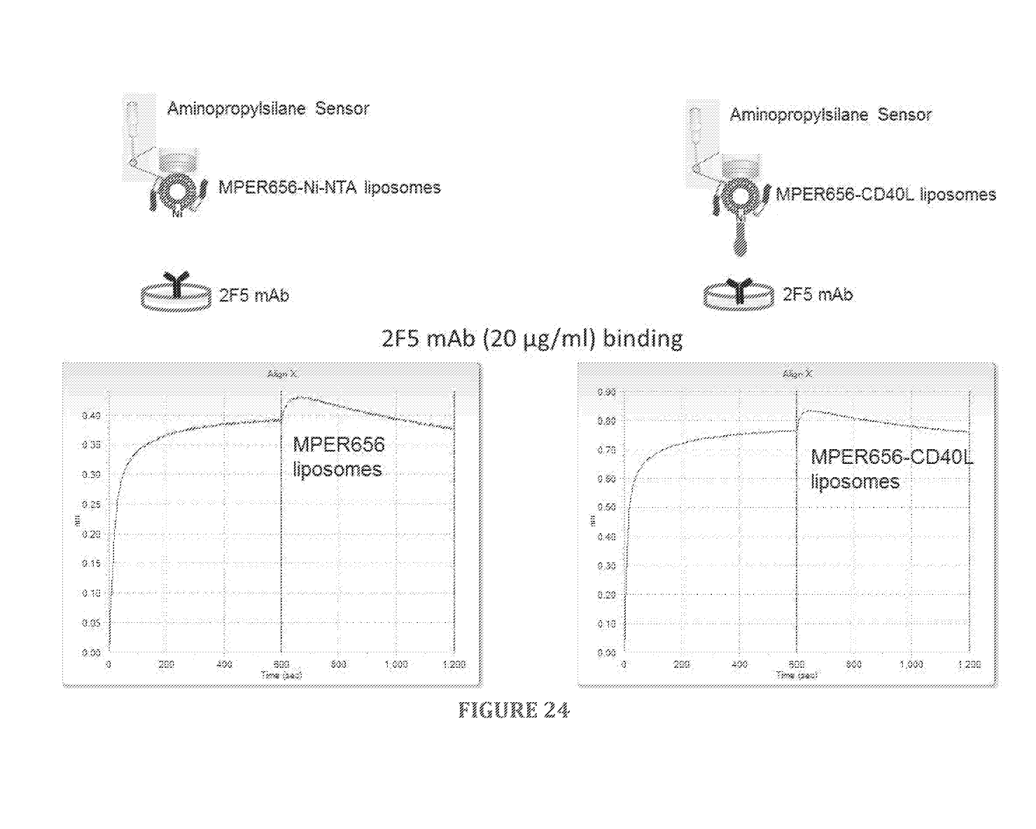

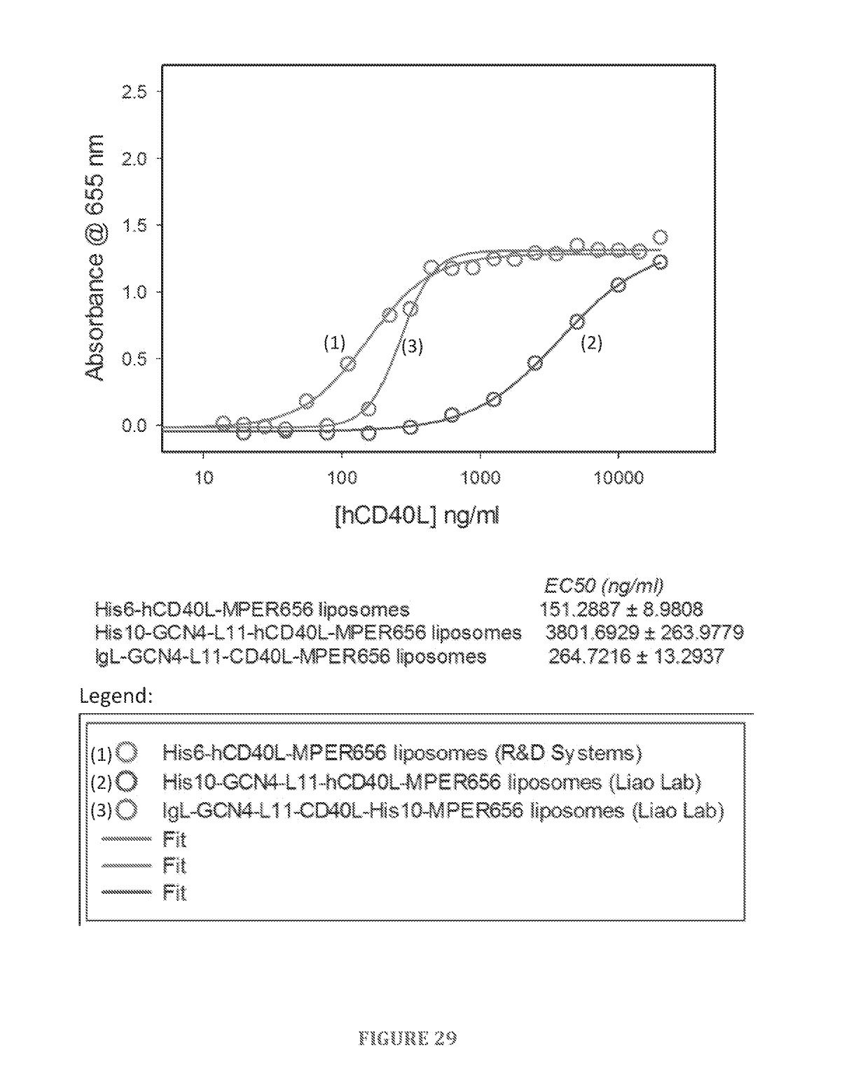

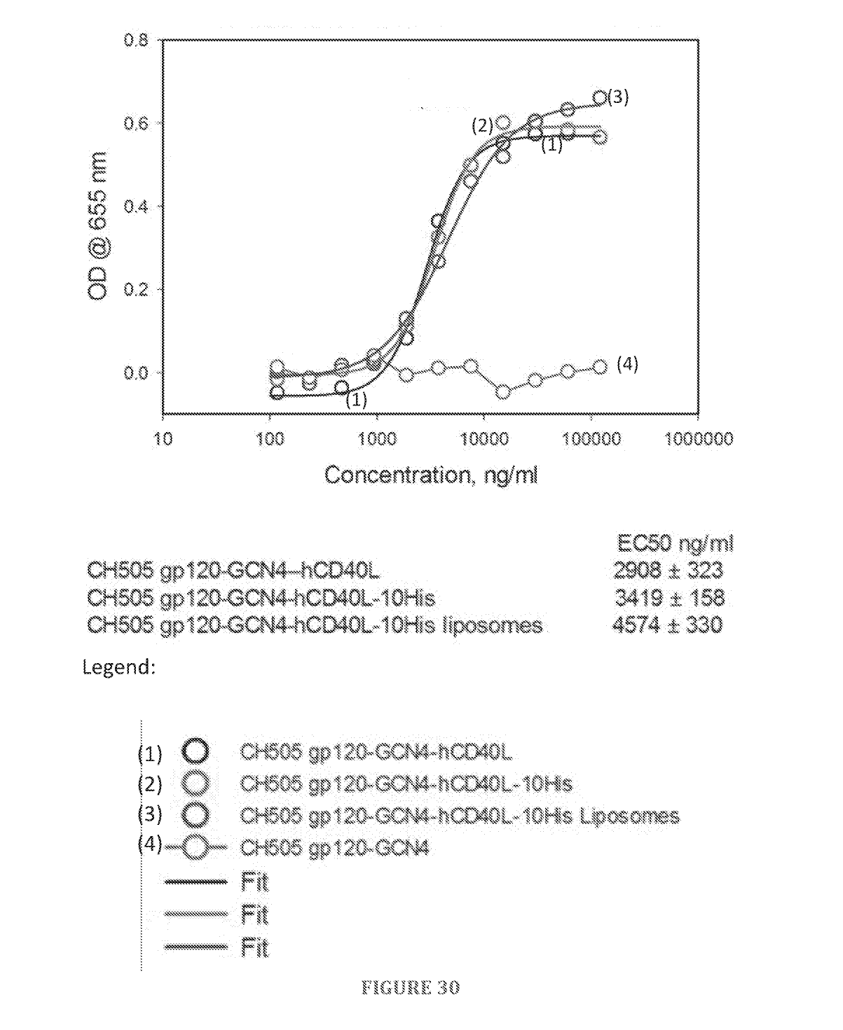

CD40L containing immunogens—MPER and gp120 envelopes—activate human CD40 expressing HEK cells. FIGS. 24-30.

### Example 2—Combination of Antigens from CH505 Envelope Sequences for Immunization

Provided herein are non-limiting examples of combinations of antigens derived from CH505 envelope sequences for a swarm immunization. The selection includes priming with a virus which binds to the UCA, for example a T/F virus or another early (e.g. but not limited to week 004.3, or 004.26) virus envelope. In certain embodiments the prime could include D-loop variants. In certain embodiments the boost could include D-loop variants.

Non-limiting embodiments of envelopes selected for swarm vaccination are shown as the selections described below. A skilled artisan would appreciate that a vaccination protocol can include a sequential immunization starting with the “prime” envelope(s) and followed by sequential boosts, which include individual envelopes or combination of envelopes. In another vaccination protocol, the sequential immunization starts with the “prime” envelope(s) and is followed with boosts of cumulative prime and/or boost envelopes. In certain embodiments, the prime does not include T/F sequence (W000.TF). In certain embodiments, the prime includes w004.03 envelope. In certain embodiments, the prime includes w004.26 envelope. In certain embodiments, the immunization methods do not include immunization with HIV-1 envelope T/F. In other embodiments for example the T/F envelope may not be included when w004.03 or w004.26 envelope is included. In certain embodiments, there is some variance in the immunization regimen; in some embodiments, the selection of HIV-1 envelopes may be grouped in various combinations of primes and boosts, either as nucleic acids, proteins, or combinations thereof.

In certain embodiments the immunization includes a prime administered as DNA, and MVA boosts. See Goepfert, et al. 2014; “Specificity and 6-Month Durability of Immune Responses Induced by DNA and Recombinant Modified Vaccinia Ankara Vaccines Expressing HIV-1 Virus-Like Particles” J Infect Dis. 2014 Feb. 9. [Epub ahead of print].

HIV-1 Envelope Selection A (Ten Envelopes Sensitive Envelopes)):

703010505.TF, 703010505.W4.03, 703010505.W4.26, 703010505.W14.21, 703010505.W20.14, 703010505.W30.28, 703010505.W30.13, 703010505.W53.31, 703010505.W78.15, 703010505.W100.B4, optionally in certain embodiments designed as trimers. See U.S. Provisional Application No. 62/027,427 incorporated by reference.

HIV-1 Envelope Selection B (Twenty Envelopes Sensitive Envelopes):

703010505.TF, 703010505.W4.03, 703010505.W4.26, 703010505.W14.3, 703010505.W14.8, 703010505.W14.21, 703010505.W20.7, 703010505.W20.26, 703010505.W20.9, 703010505.W20.14, 703010505.W30.28, 703010505.W30.12, 703010505.W30.19, 703010505.W30.13, 703010505.W53.19, 703010505.W53.13, 703010505.W53.31, 703010505.W78.1, 703010505.W78.15, 703010505.W100.B4, optionally in certain embodiments designed as trimers. See U.S. Provisional Application No. 62/027,427 incorporated by reference.

HIV-1 Envelope Selection C (Four Envelopes):

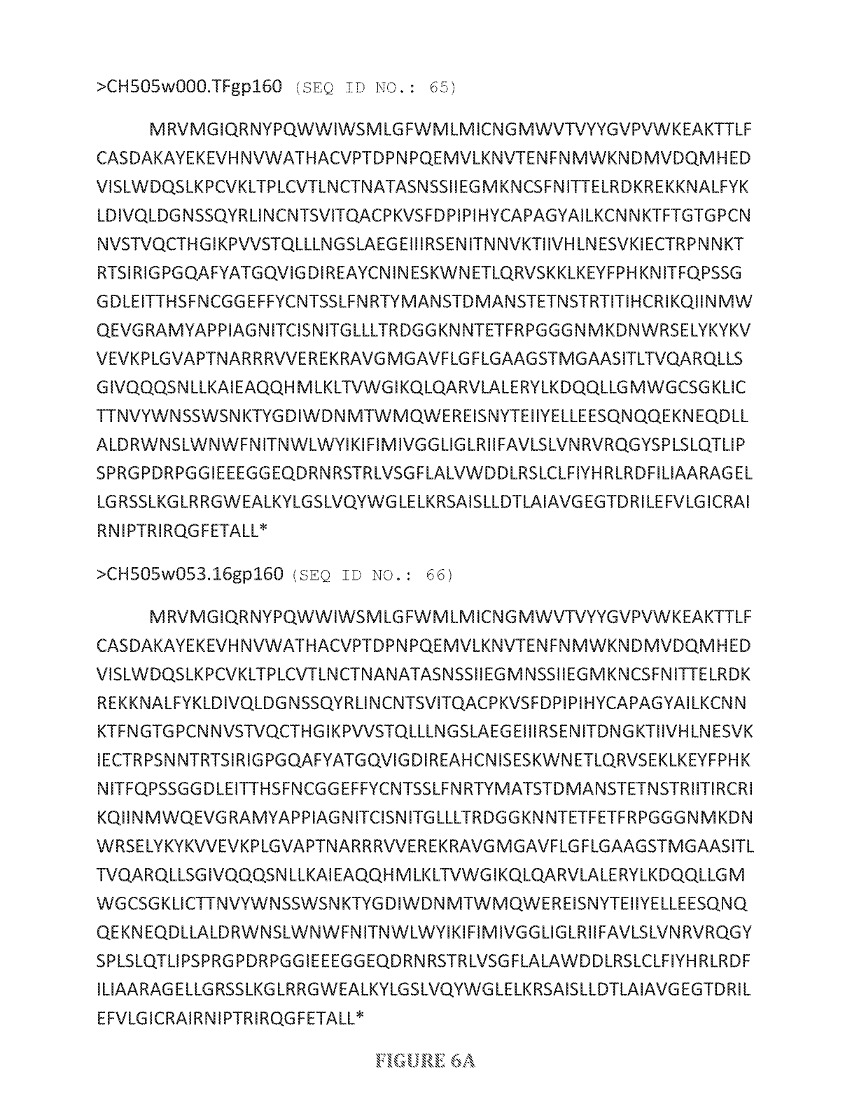

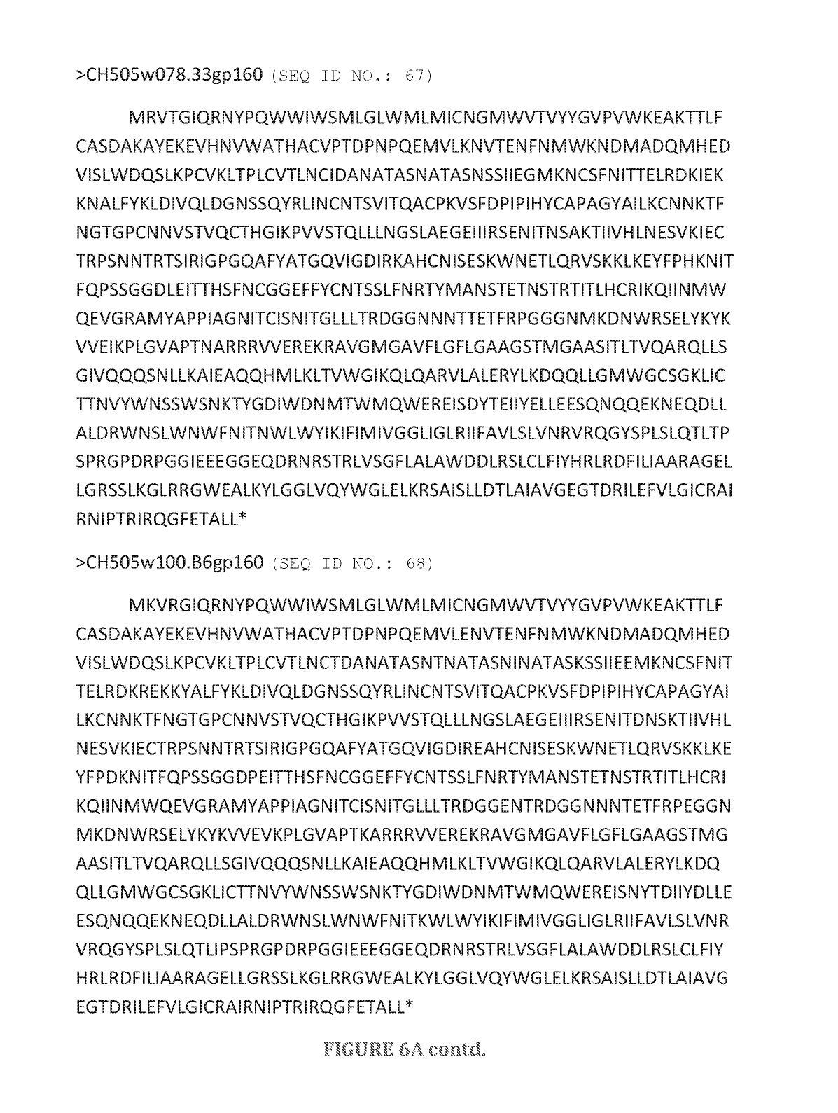

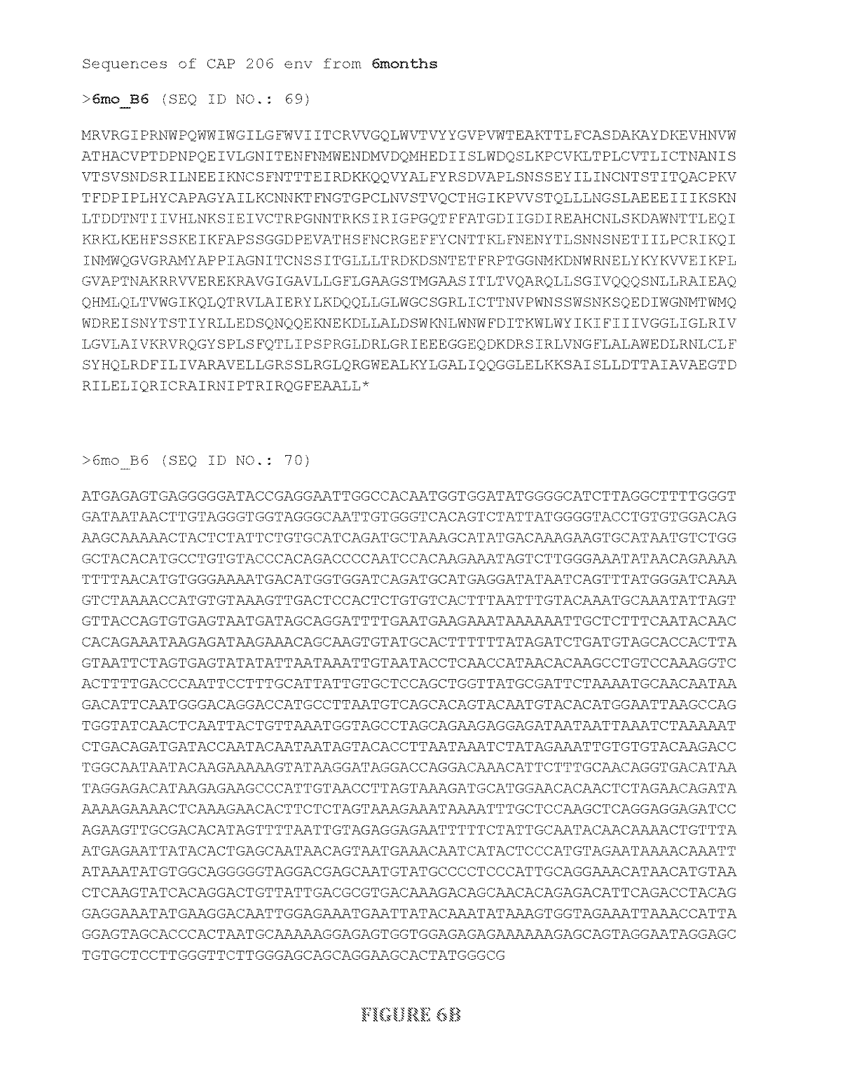

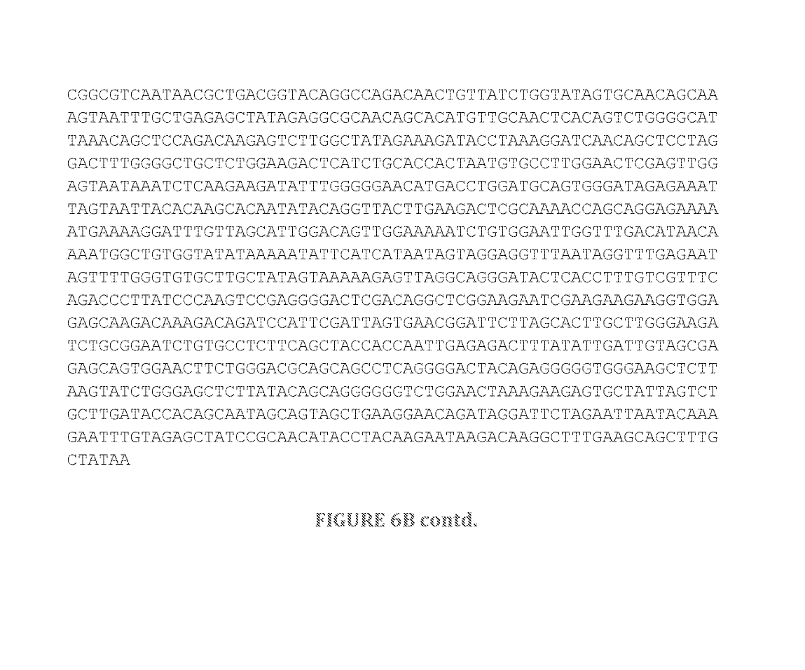

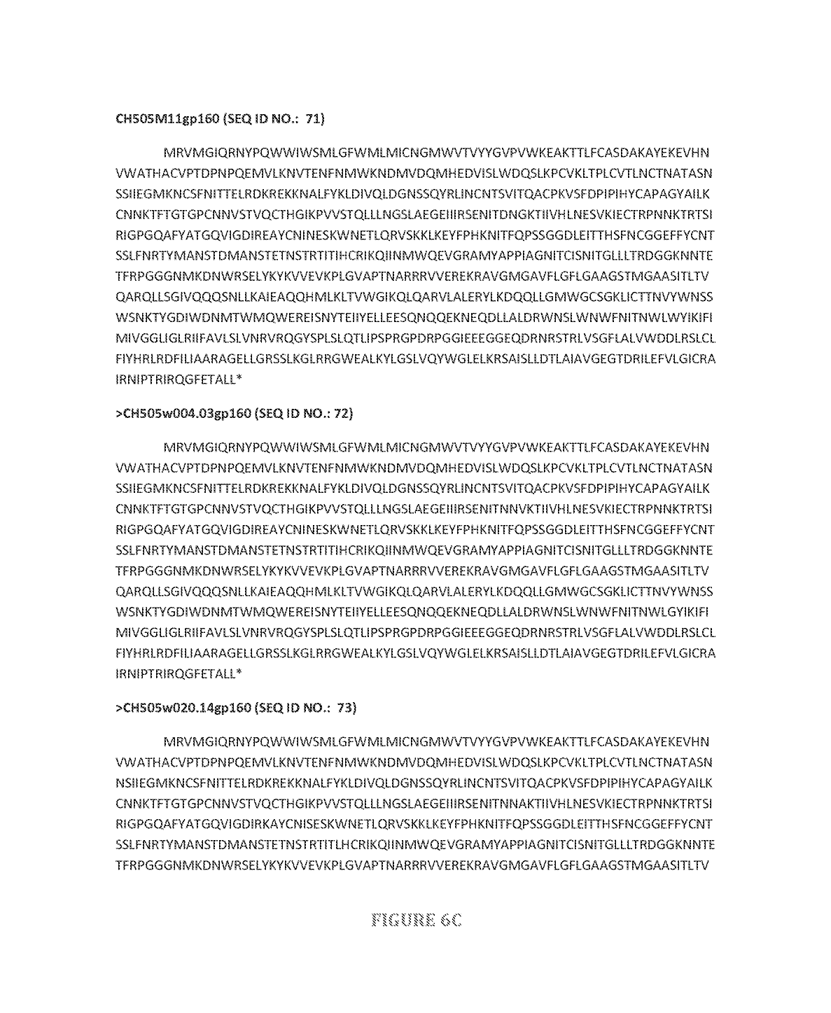

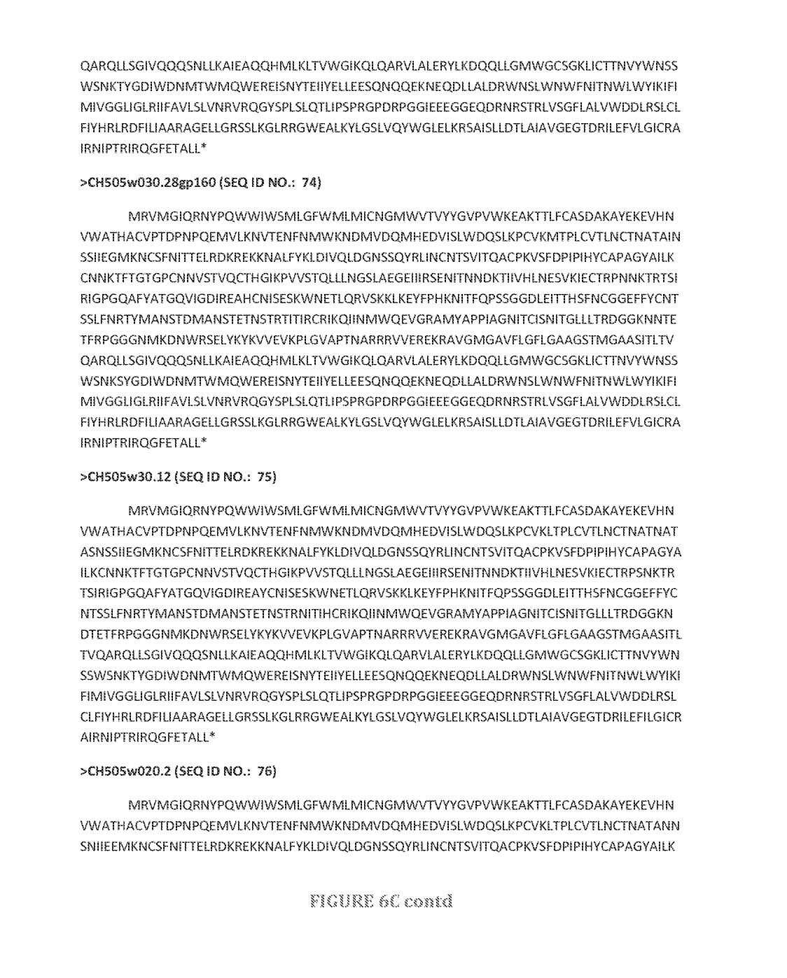

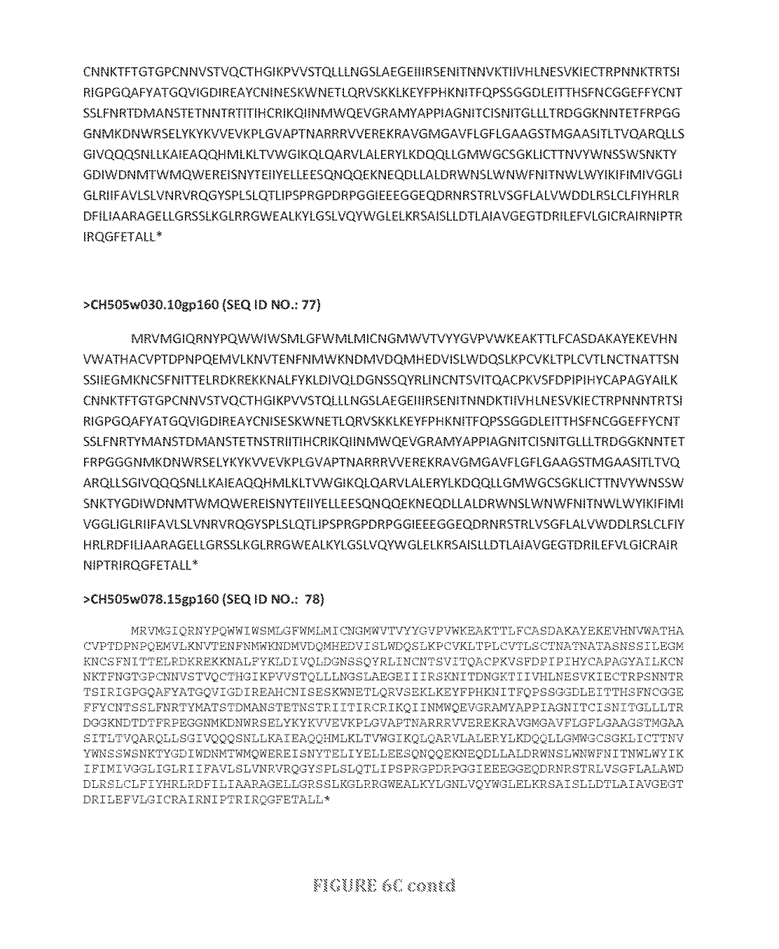

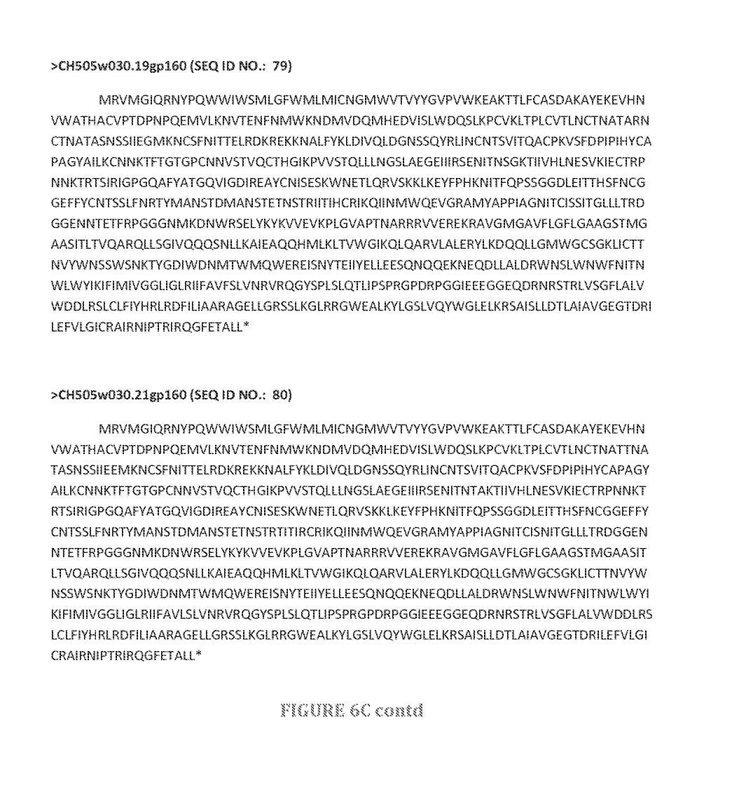

703010505.TF, 703010505.W53.16, 703010505.W78. 33, 703010505.W100.B6, optionally in certain embodiments designed as trimers. See FIG. 6A; See WO2014042669.

HIV-1 Envelope Selection D (Ten Production Envelopes):

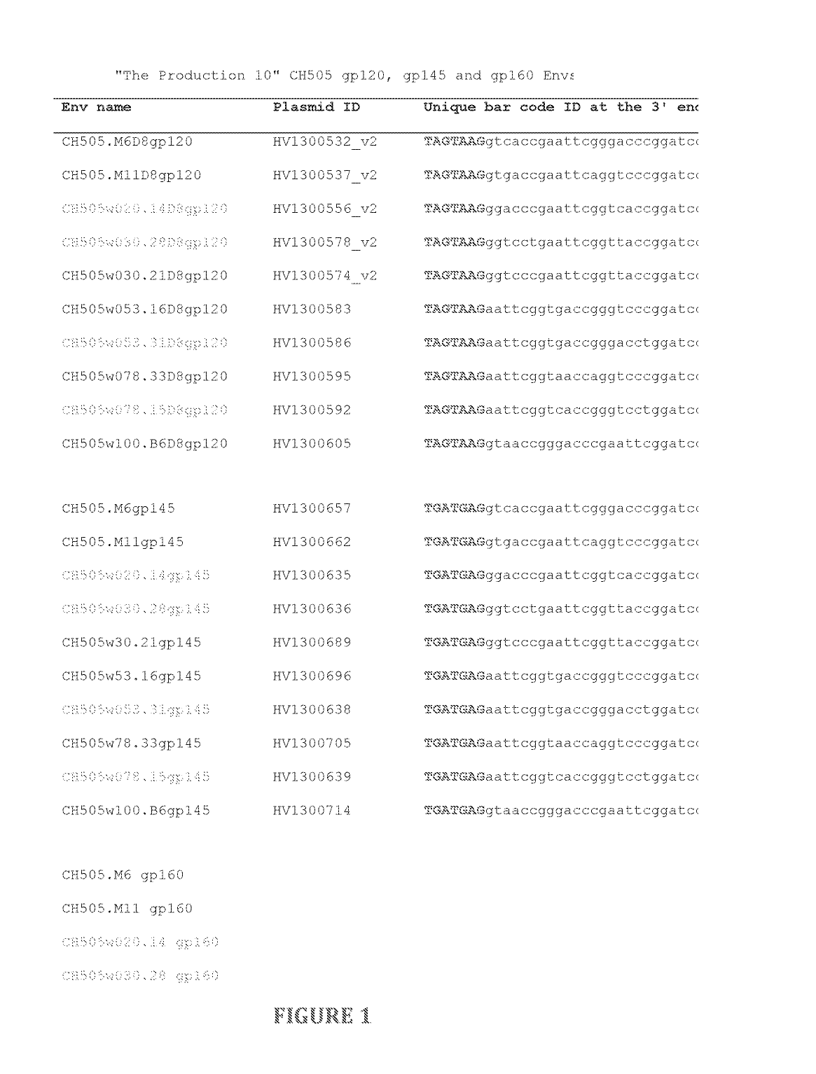

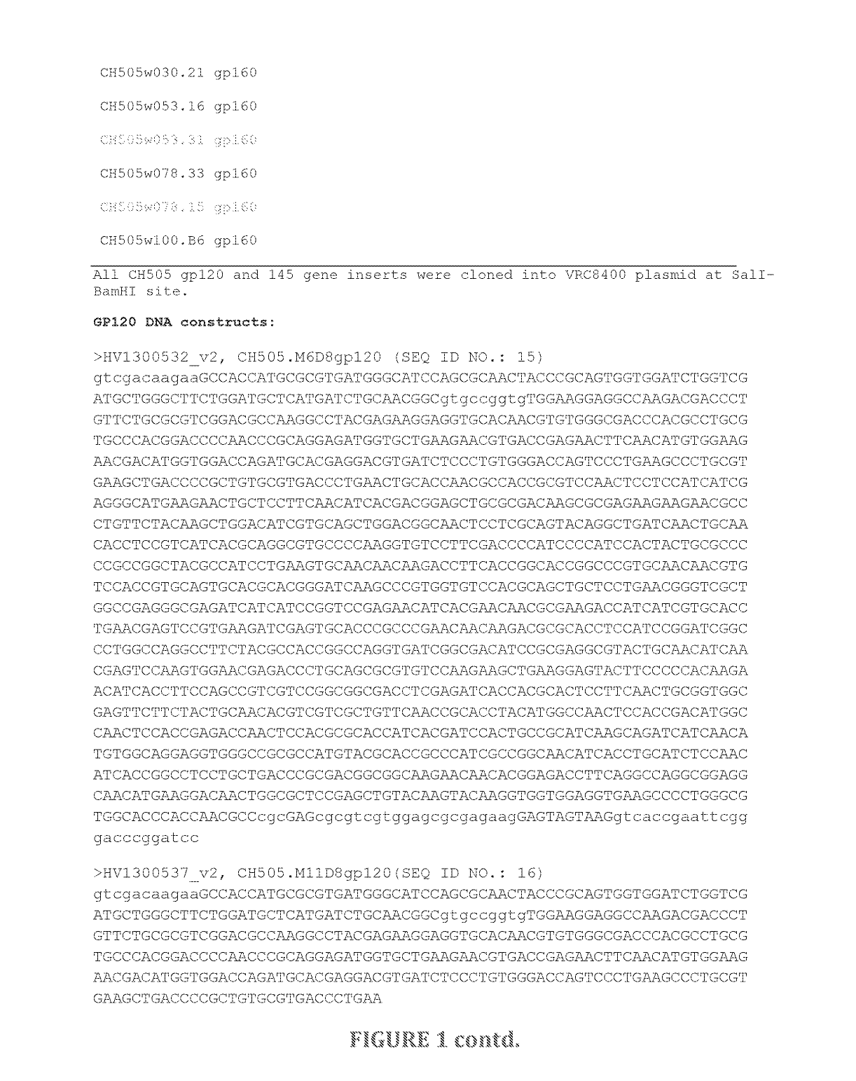

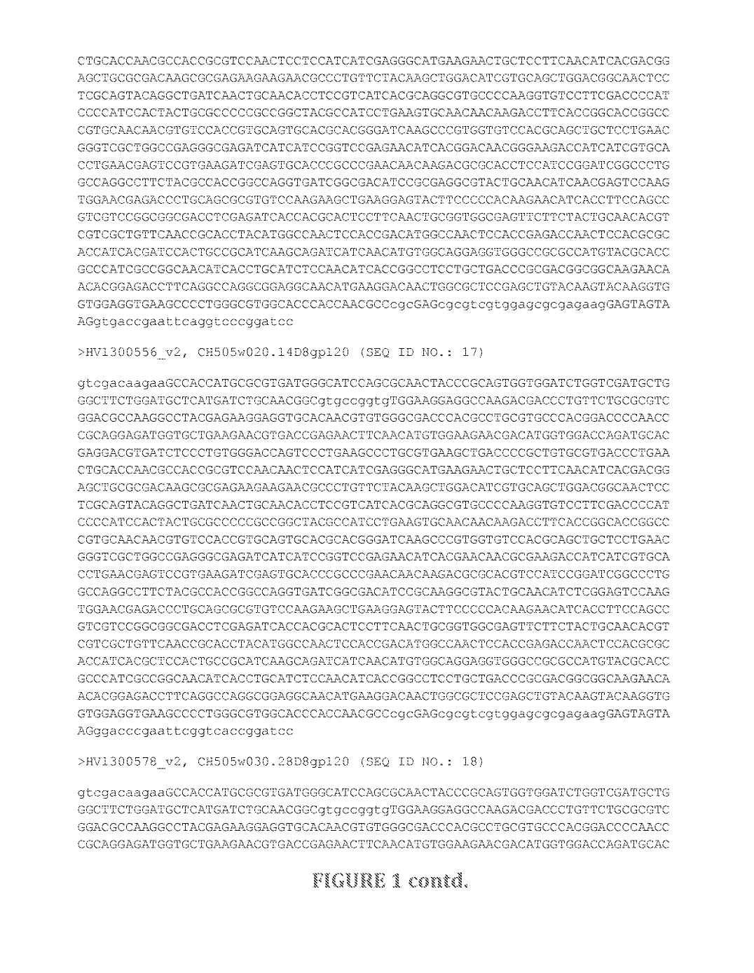

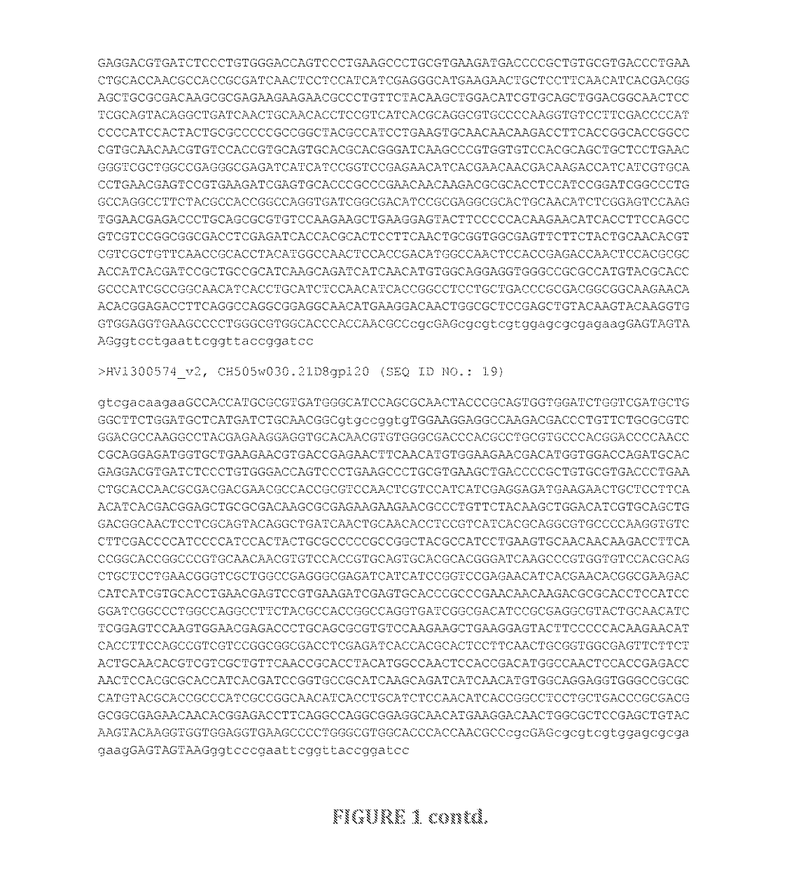

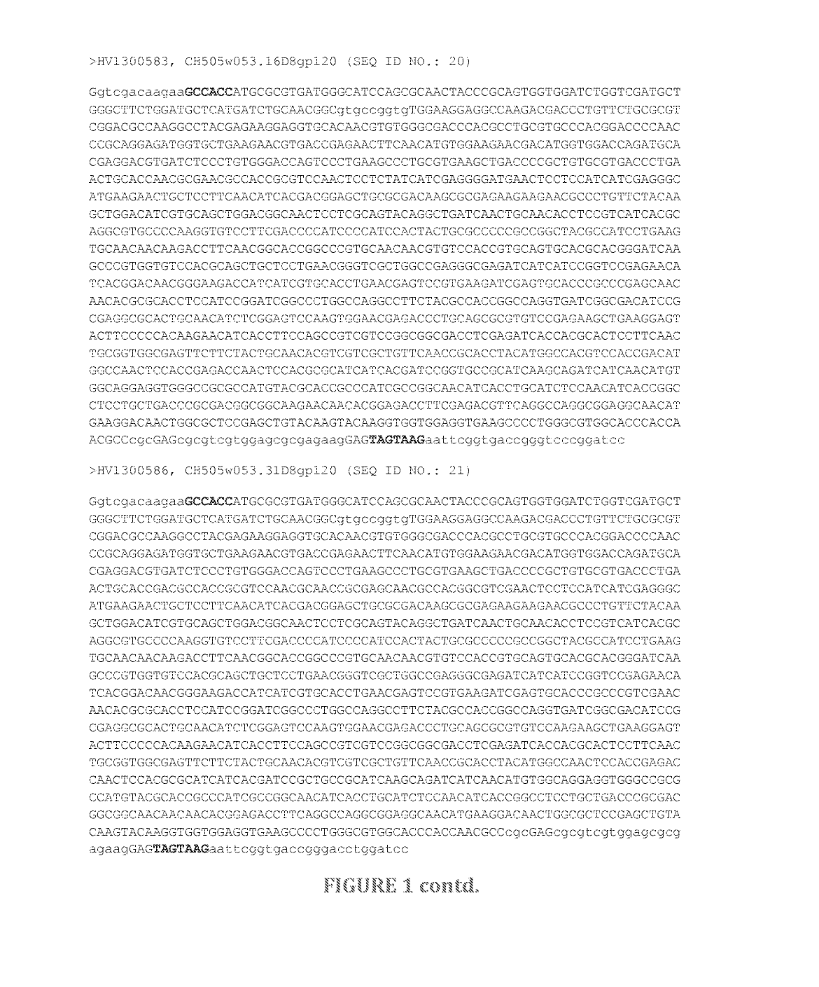

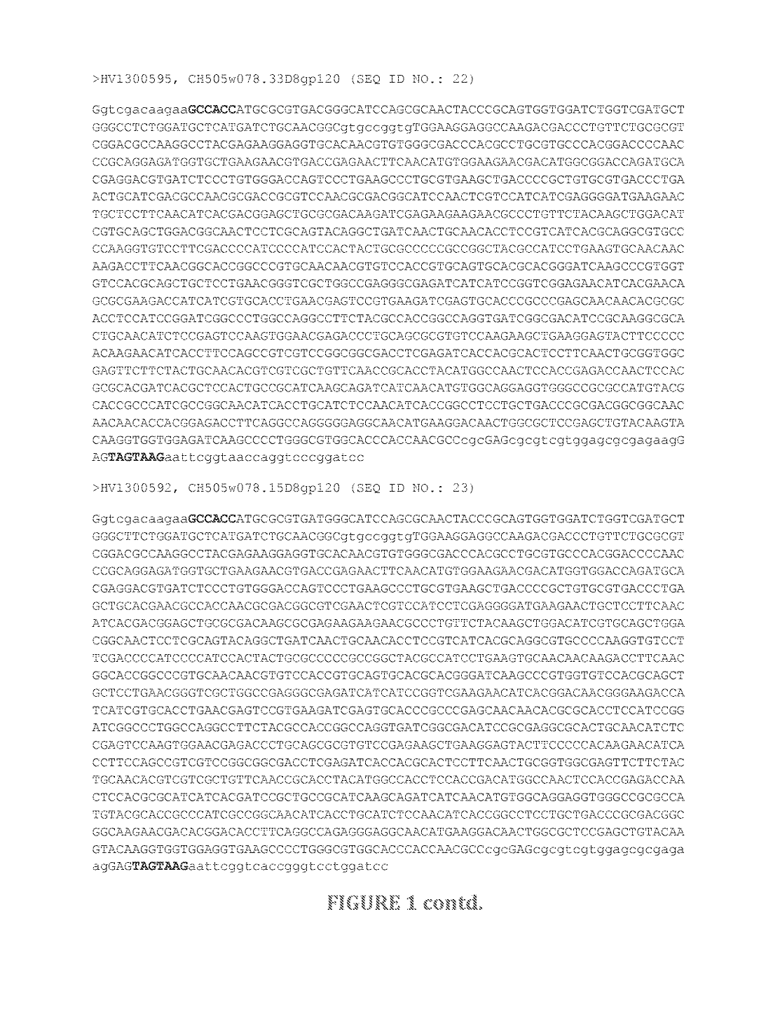

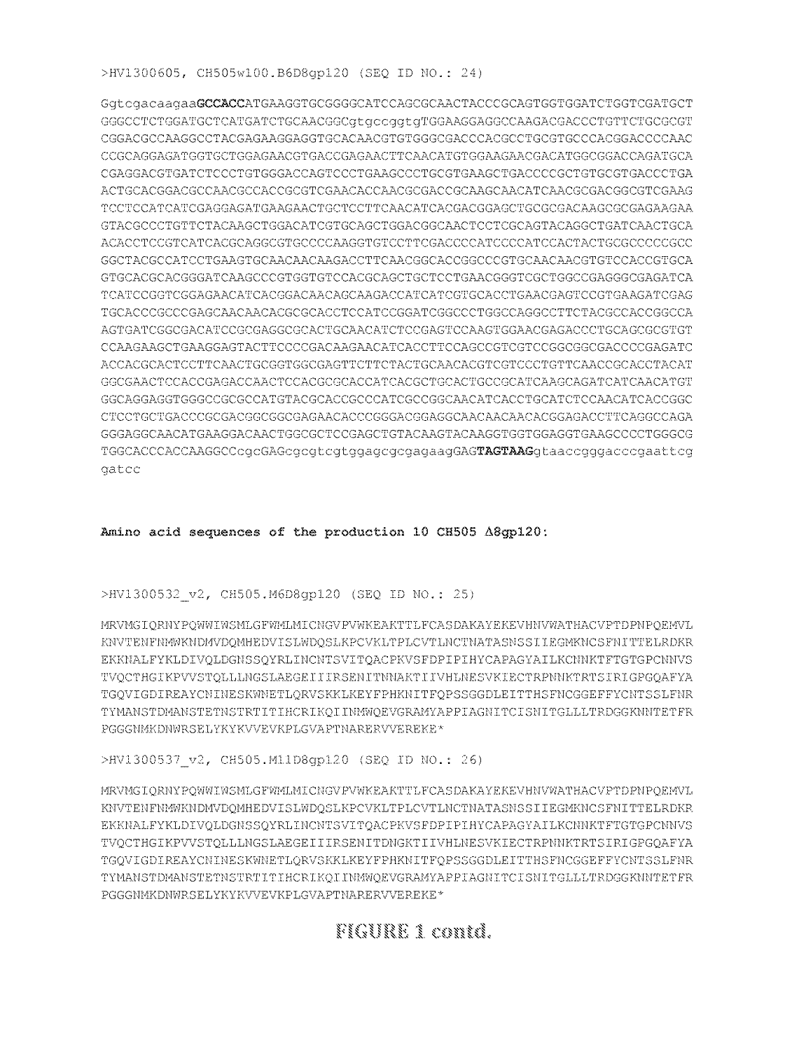

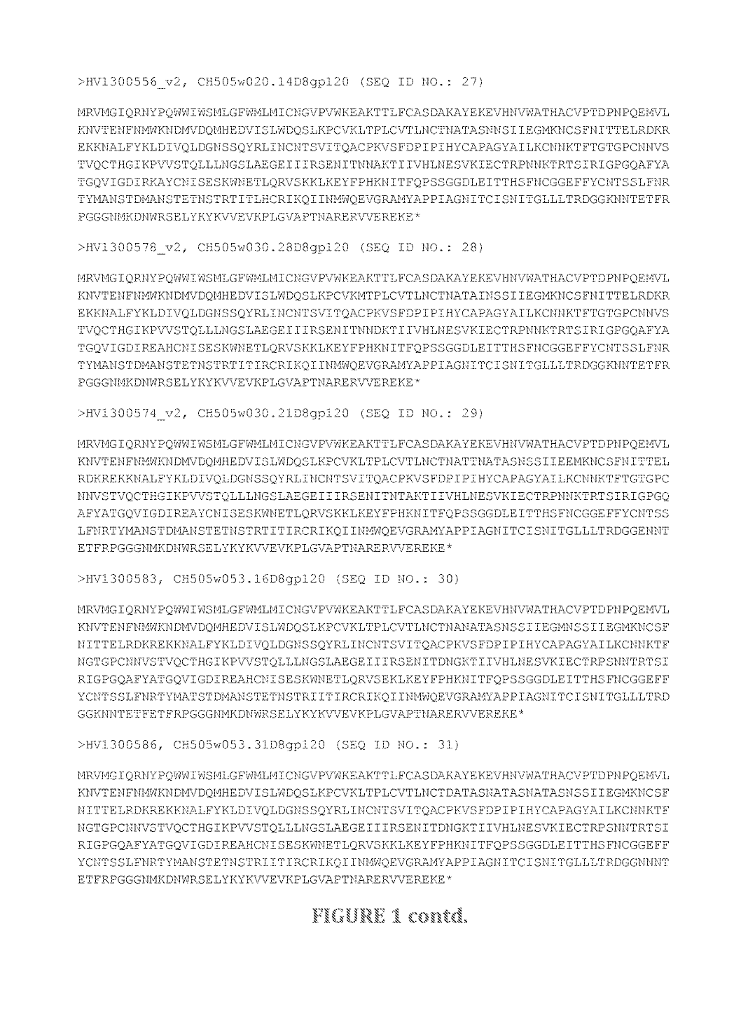

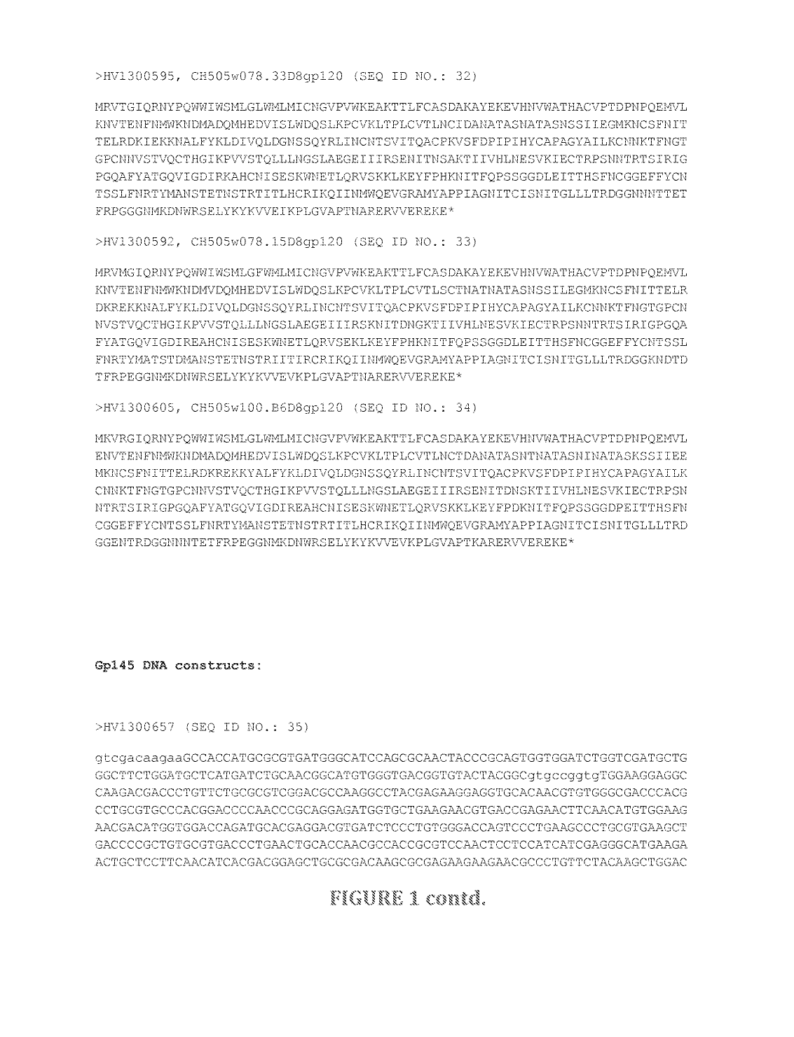

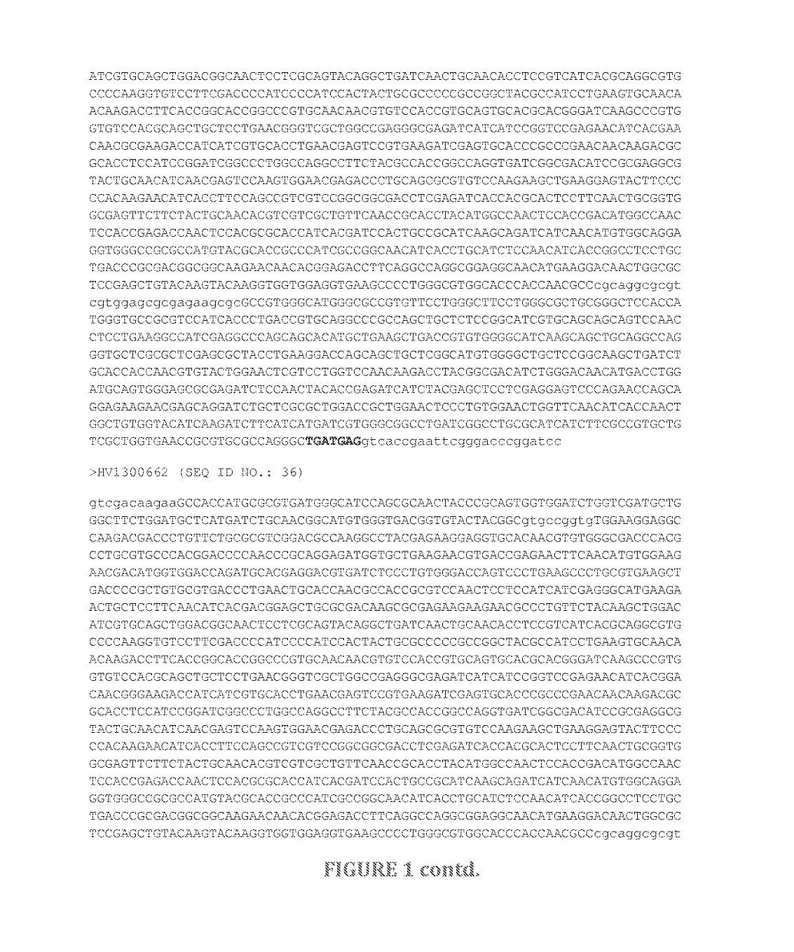

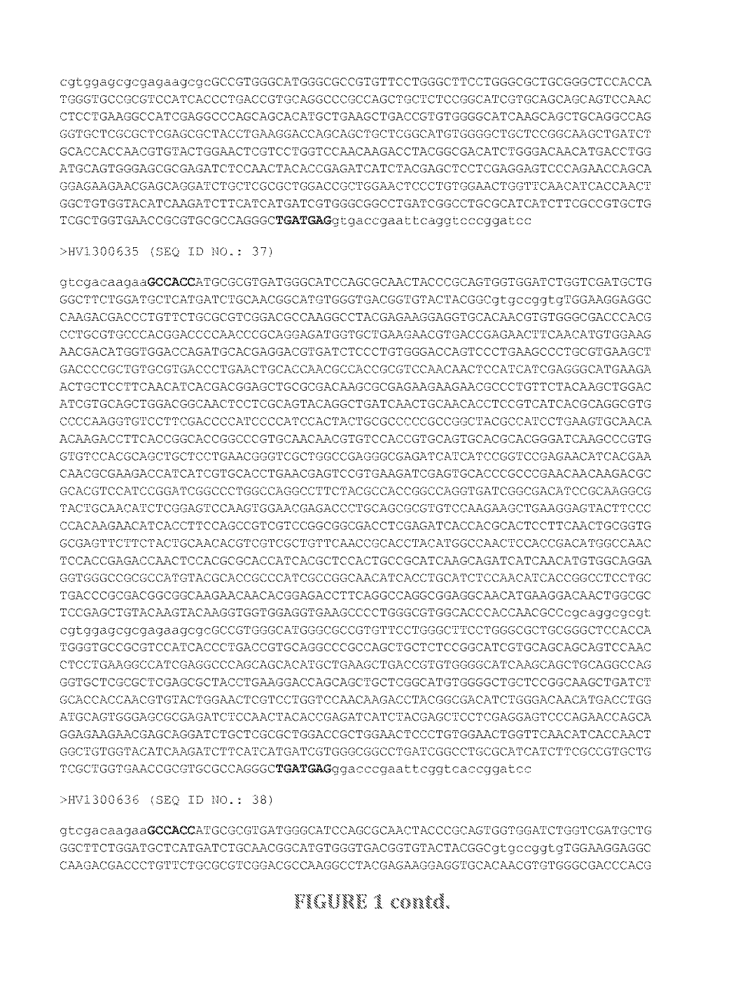

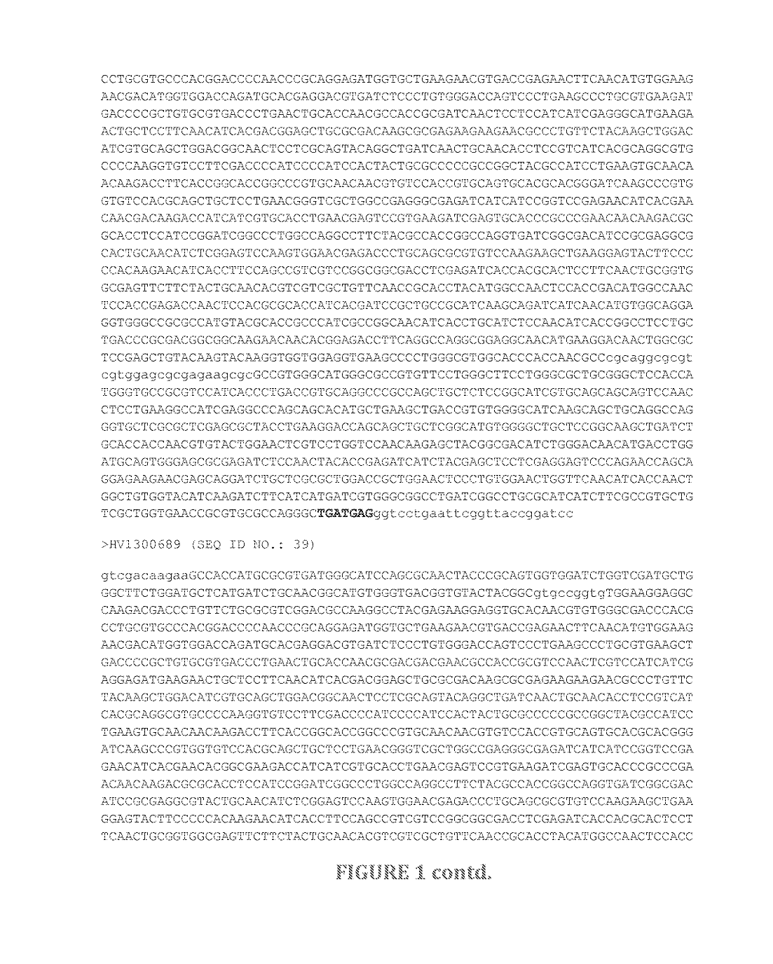

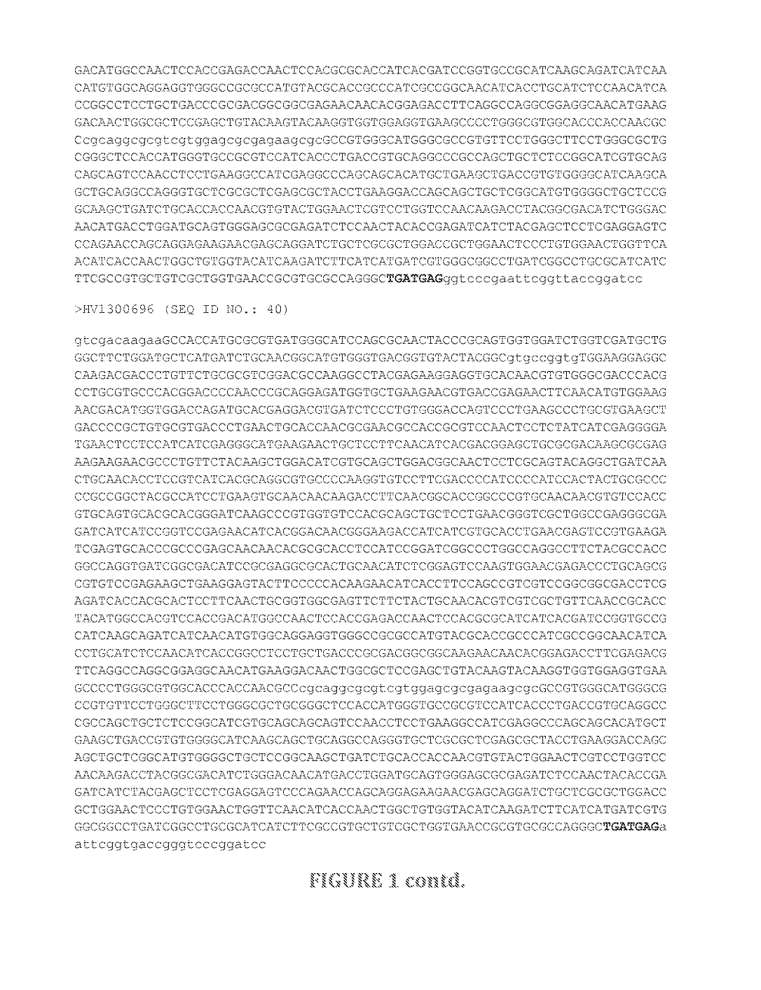

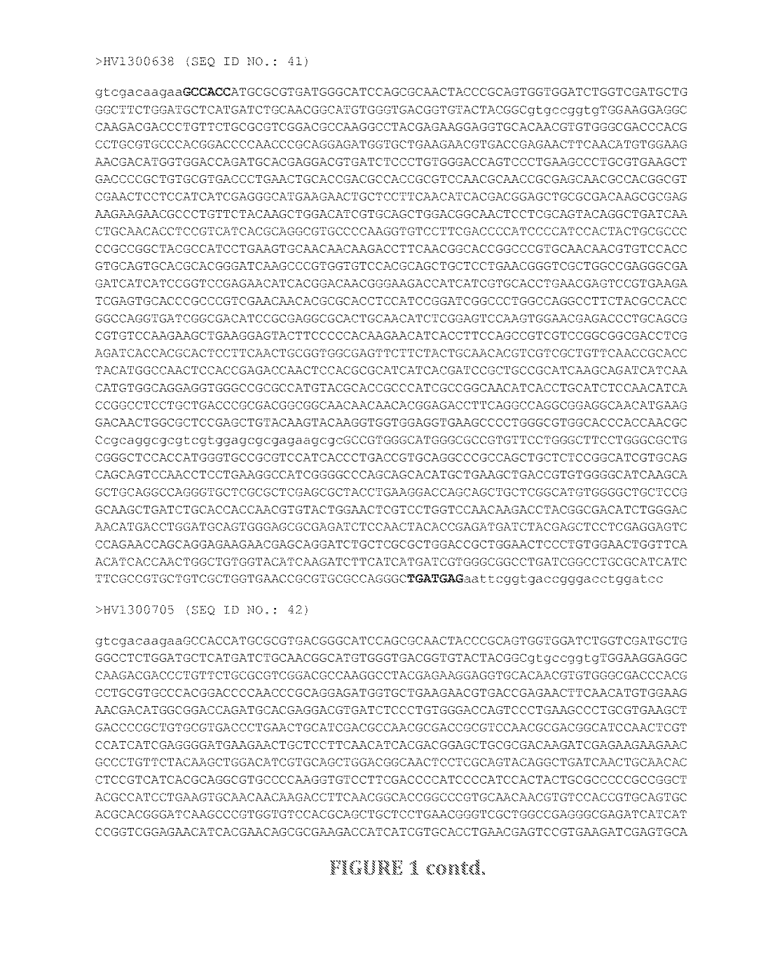

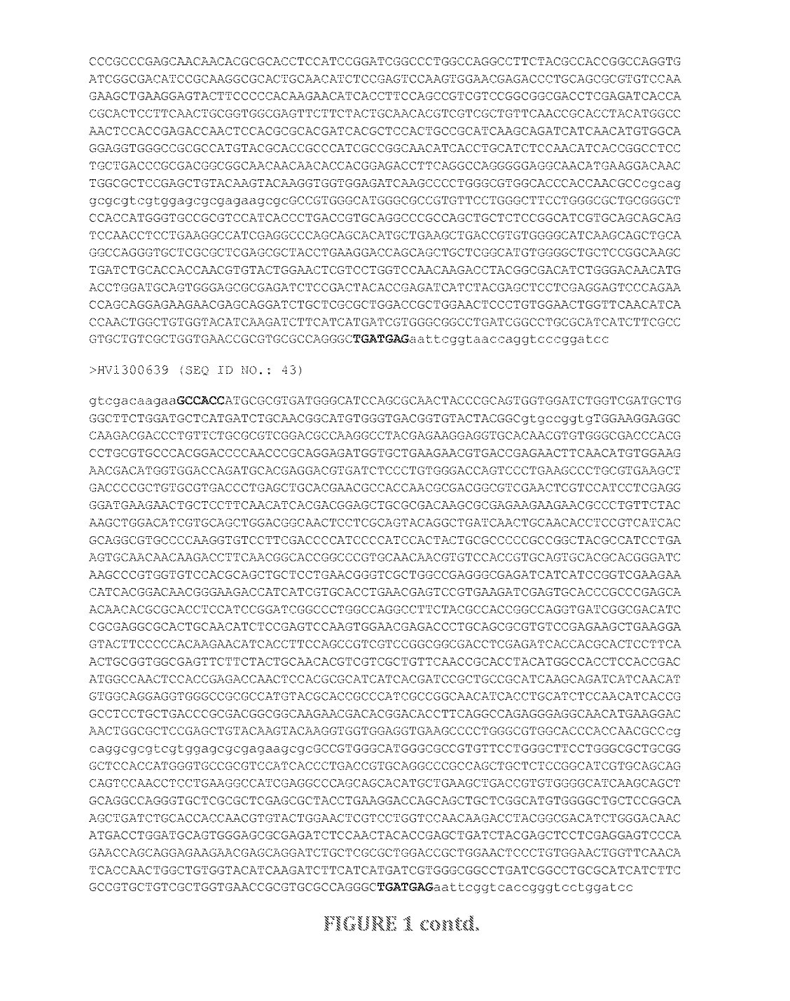

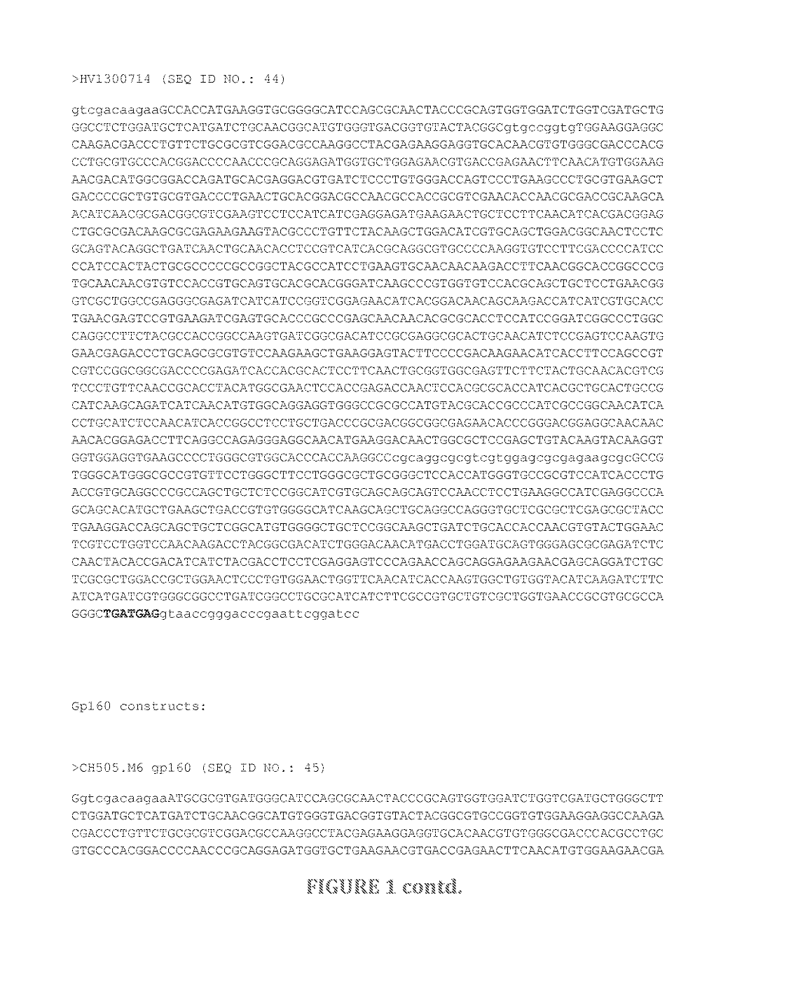

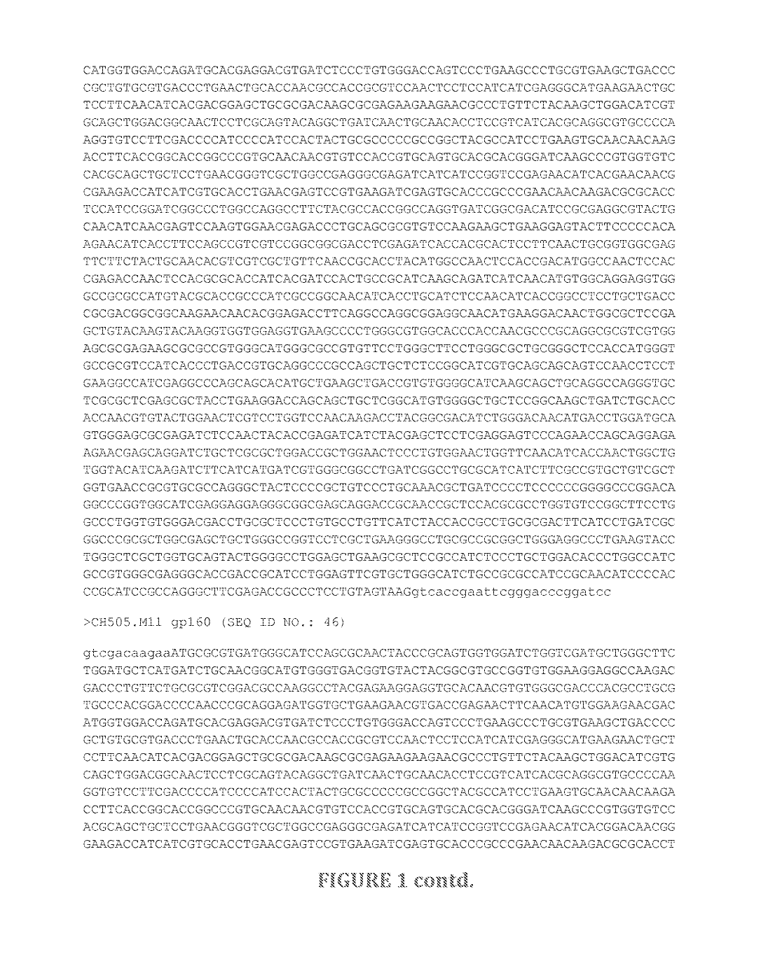

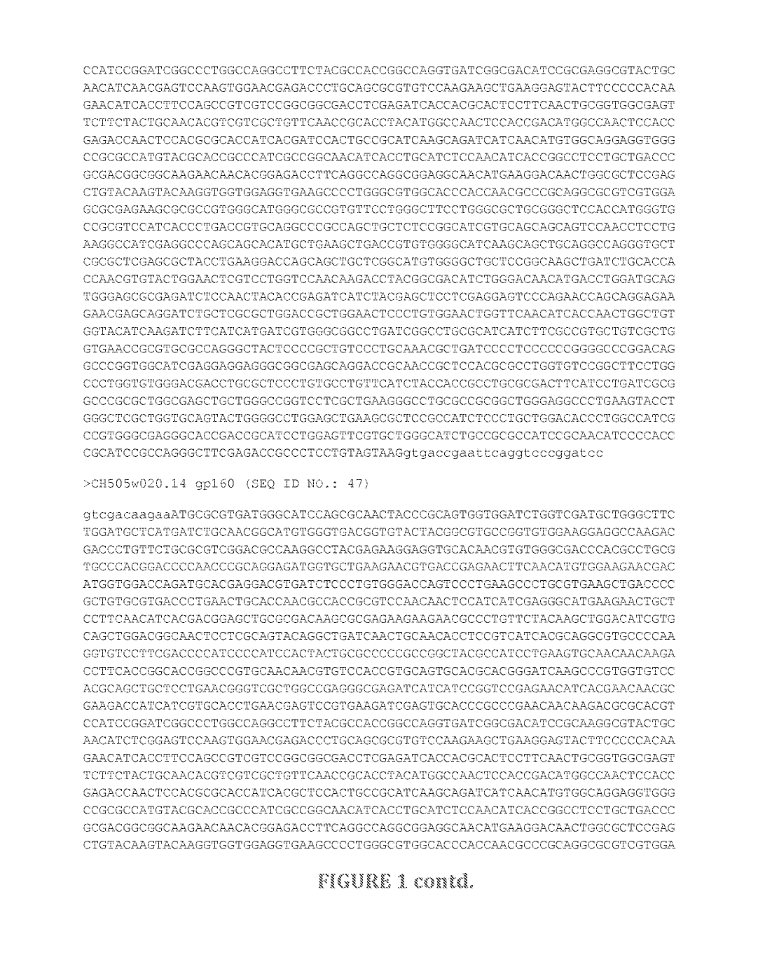

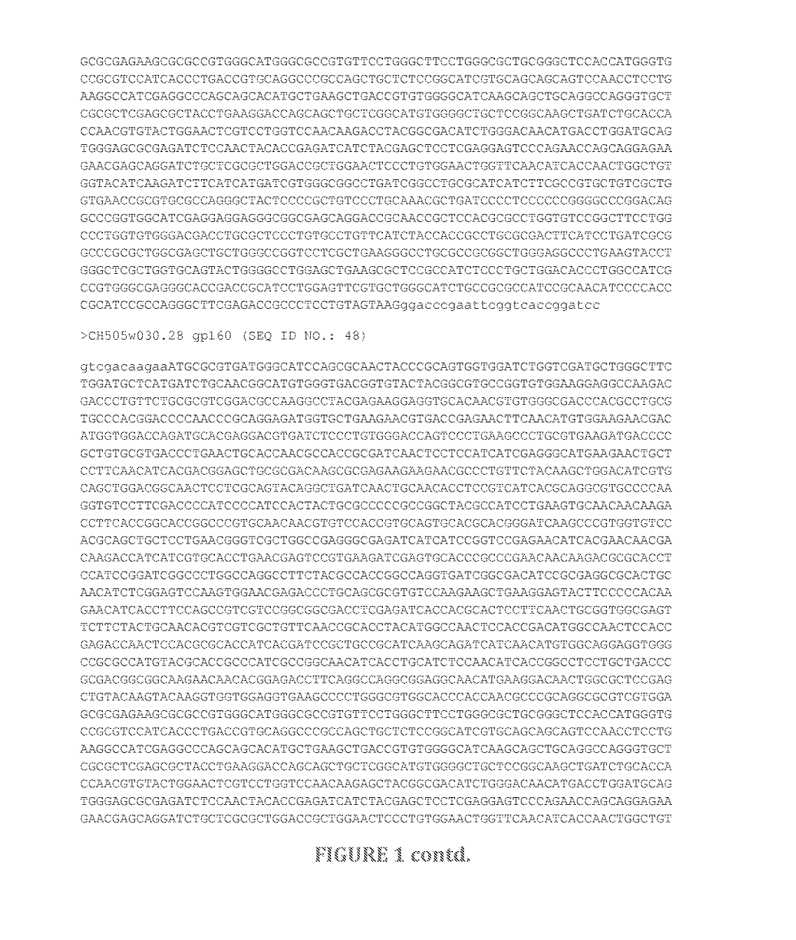

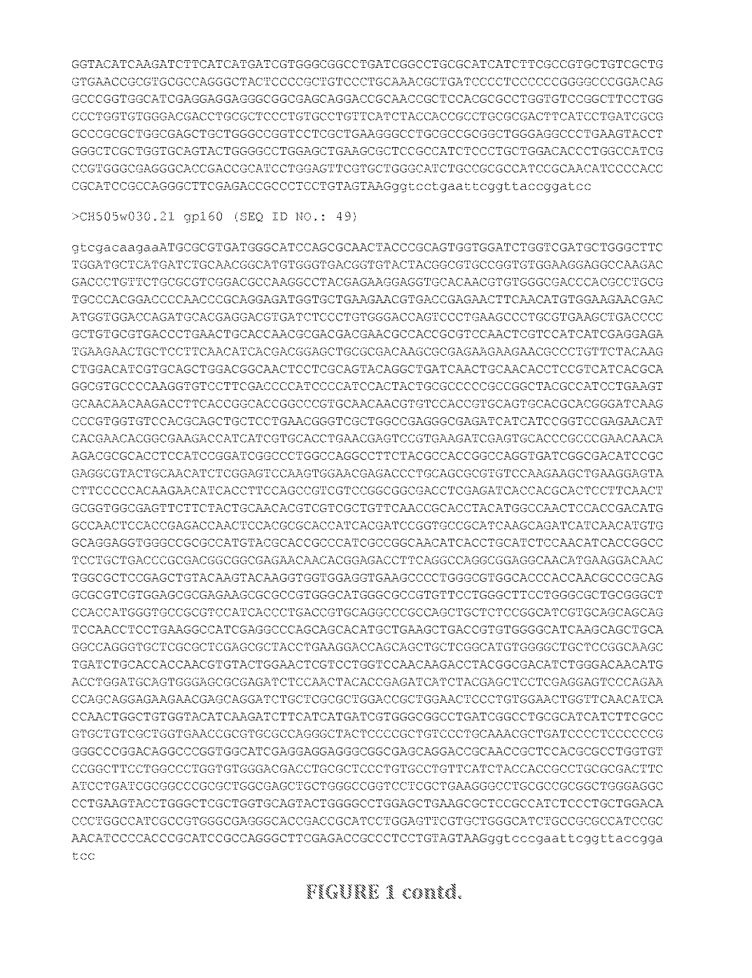

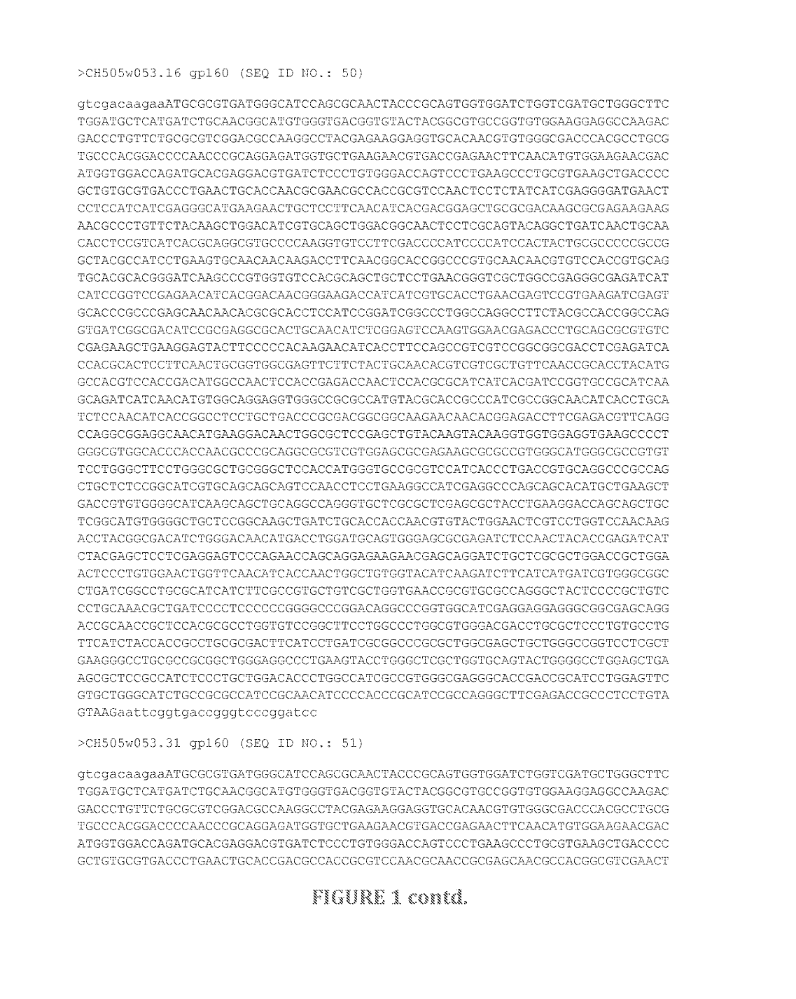

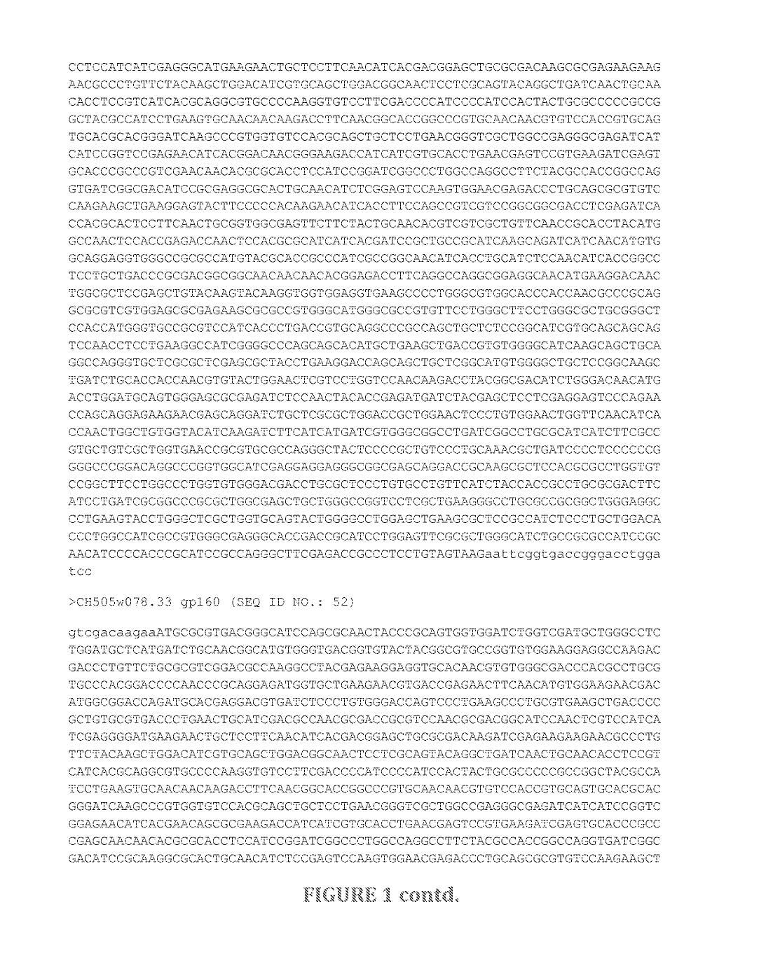

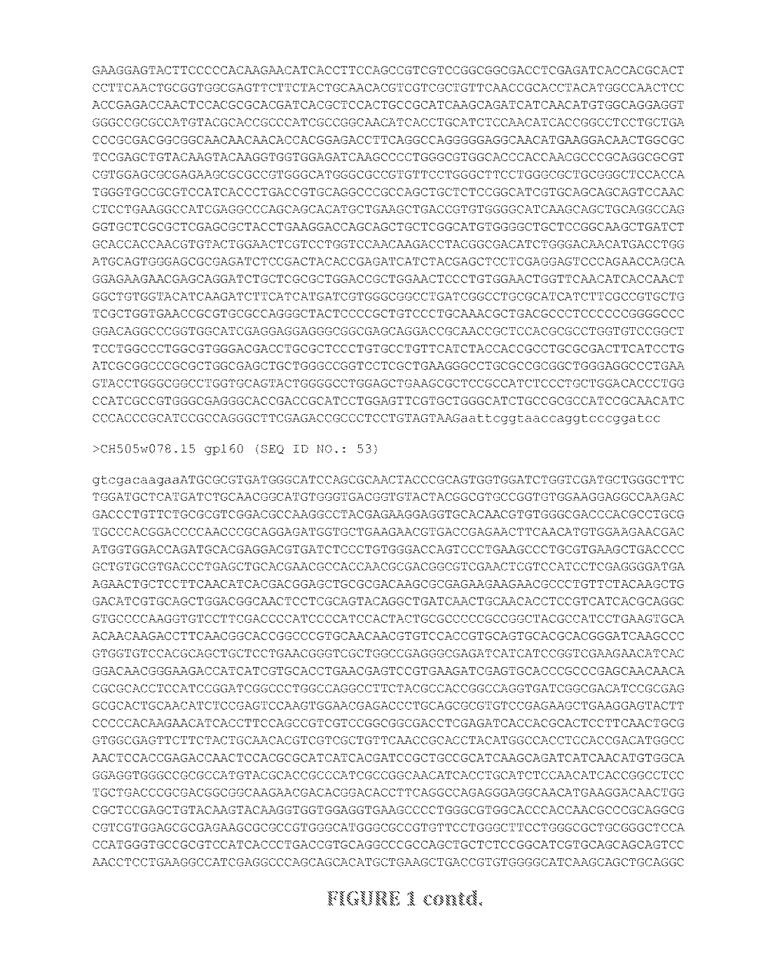

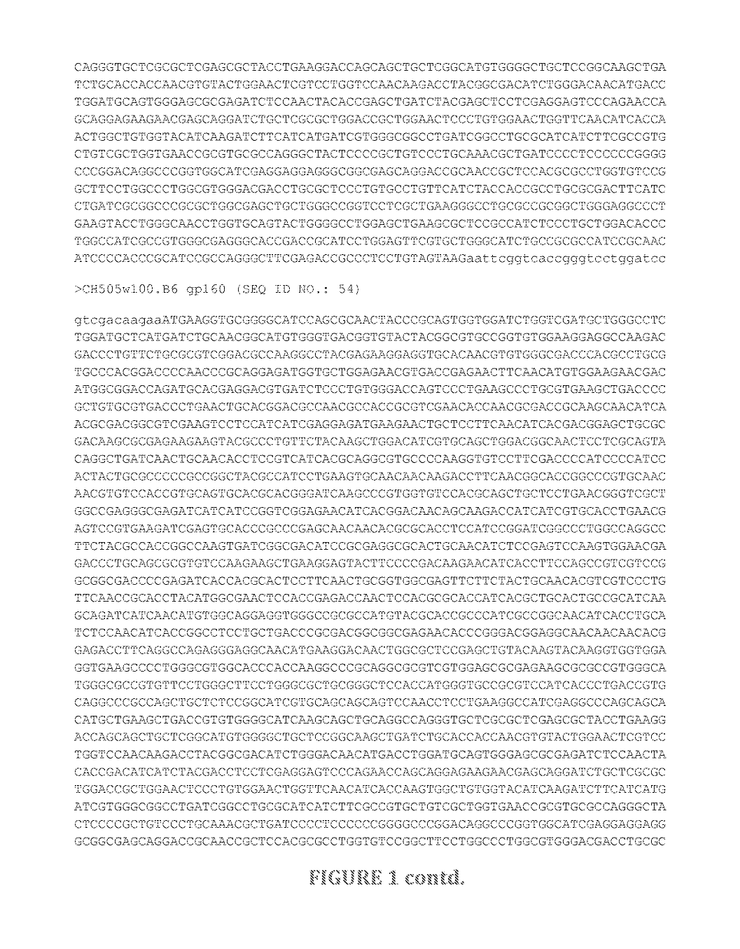

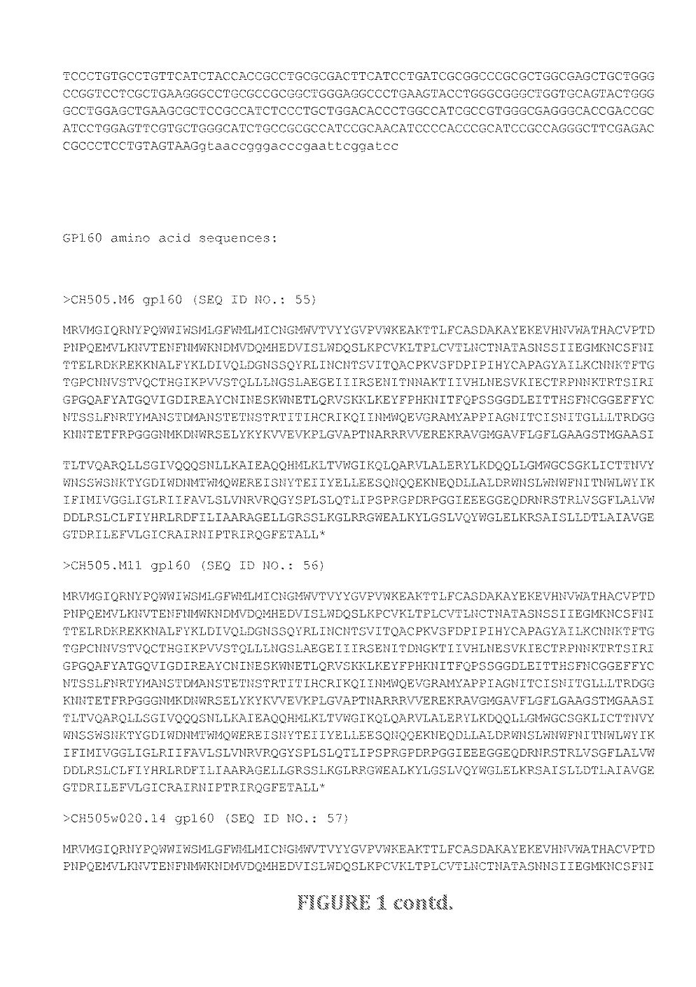

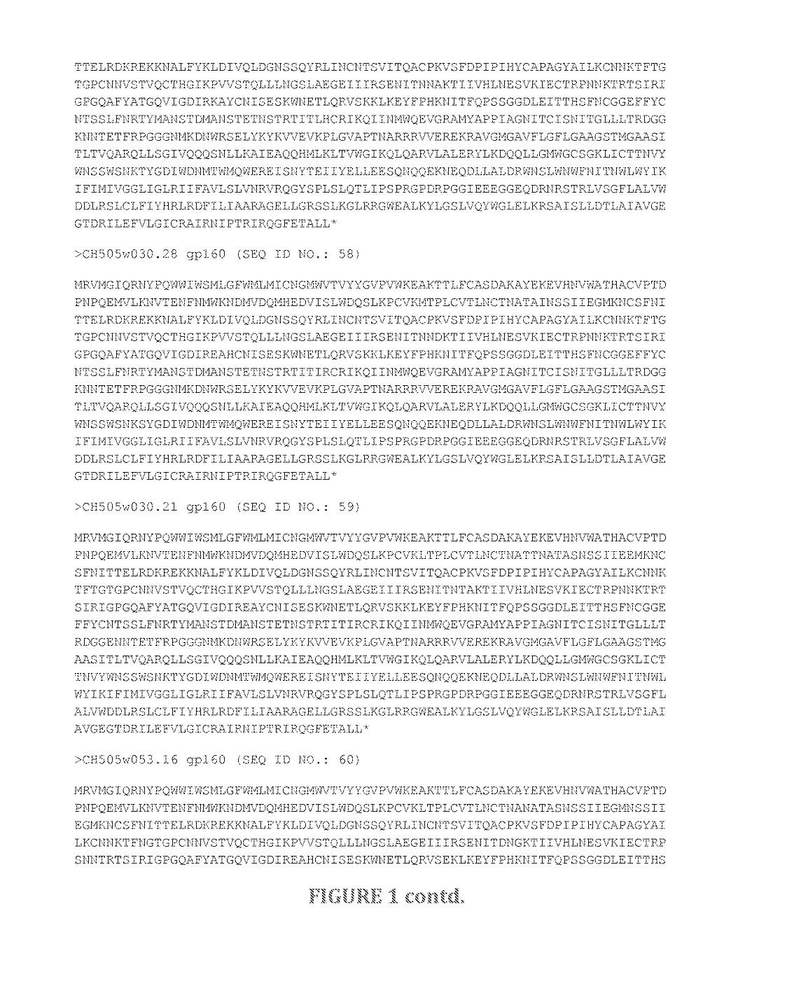

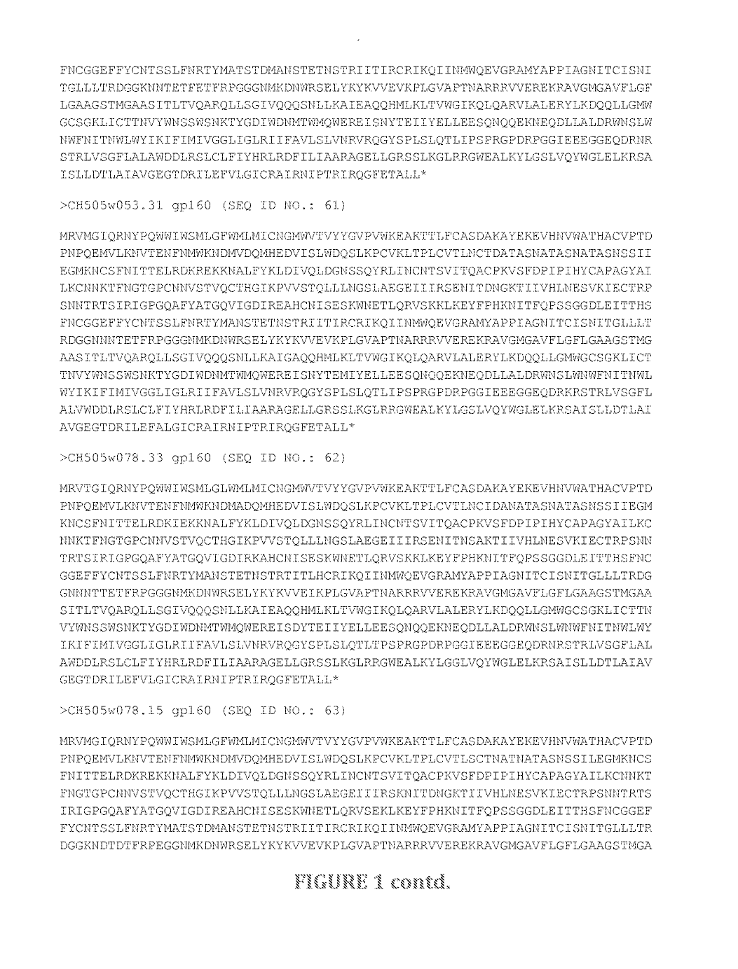

CH505.M6; CH505.M11; CH505w020.14; CH505w030.28; CH505w030.21; CH505w053.16; CH505w053.31; CH505w078.33; CH505w078.15; CH505w100.B6, optionally in certain embodiments designed as trimers. See FIG. 1.

HIV-1 Envelopes Selection E (Ten Early Envelopes):

CH505.M11; CH505.w004.03; CH505.w020.14; CH505.w030.28; CH505.w030.12; CH505.w020.2; CH505.w030.10; CH505.w078.15; CH505.w030.19; CH505.w030.21, optionally in certain embodiments designed as trimers. See FIG. 6C.

HIV-1 Selection F(10PR):

CH505.T/F; CH505.M11; CH505w020.14; CH505w030.28; CH505w030.21; CH505w053.16; CH505w053.31; CH505w078.33; CH505w078.15; CH505w100.B.

### Example 3: Examples of Immunization Protocols in Subjects with Swarms of HIV-1 Envelopes

Immunization protocols contemplated by the invention include envelopes sequences as described herein including but not limited to nucleic acids and/or amino acid sequences of gp160s, gp150s, cleaved and uncleaved gp140s, gp120s, gp41s, N-terminal deletion variants as described herein, cleavage resistant variants as described herein, or codon optimized sequences thereof. A skilled artisan can readily modify the gp160 and gp120 sequences described herein to obtain these envelope variants. The swarm immunization protocols can be administered in any subject, for example monkeys, mice, guinea pigs, or human subjects.

In non-limiting embodiments, the immunization includes a nucleic acid is administered as DNA, for example in a modified vaccinia vector (MVA). In non-limiting embodiments, the nucleic acids encode gp160 envelopes. In other embodiments, the nucleic acids encode gp120 envelopes. In other embodiments, the boost comprises a recombinant gp120 envelope. The vaccination protocols include envelopes formulated in a suitable carrier and/or adjuvant, for example but not limited to alum. In certain embodiments the immunizations include a prime, as a nucleic acid or a recombinant protein, followed by a boost, as a nucleic acid or a recombinant protein. A skilled artisan can readily determine the number of boosts and intervals between boosts.

In non-limiting embodiments, the prime includes a 703010505.TF envelope and a loop D variant as described herein. In non-limiting embodiments, the prime includes a 703010505.TF envelope and/or 703010505.W4.03, 703010505.W4.26 envelope, and a loop D variant as described herein. In certain embodiments, the loop D variant is M6. In certain embodiments, the loop D variant is M5. In certain embodiments, the loop D variant is M10. In certain embodiments, the loop D variant is M19. In certain embodiments, the loop D variant is M11. In certain embodiments, the loop D variant is M20. In certain embodiments, the loop D variant is M21. In certain embodiments, the loop D variant is M9. In certain embodiments, the loop D variant is M8. In certain embodiments, the loop D variant is M7.

Table 1 shows a non-limiting example of a sequential immunization protocol using a swarm of HIV1 envelopes (703010505.TF, 703010505.W4.03, 703010505.W4.26, 703010505.W14.21, 703010505.W20.14, 703010505.W30.28, 703010505.W30.13, 703010505.W53.31, 703010505.W78.15, 703010505.W100.B4, optionally in certain embodiments designed as trimers. In a non-limiting embodiment, a suggested grouping for prime and boost is to begin with the CH505 TF+W4.03, then boost with a mixture of w4.26+14.21+20.14, then boost with a mixture of w30.28+30.13+53.31, then boost with a mixture of w78.15+100.B4.

| Envelope | Prime | Boost(s) | Boost(s) | Boost(s) |
|---|---|---|---|---|
| CH505 TF + W4.03 | CH505 TF + W4.03 | | | |
| | as a nucleic acid | | | |
| | e.g. DNA/MVA | | | |
| | vector and/or protein | | | |
| w4.26 + 14.21 + | | w4.26 + 14.21 + | | |
| 20.14 | | 20.14 as a nucleic | | |
| | | acid e.g. DNA/MVA | | |
| | | vector and/or protein | | |
| w30.28 + 30.13 + | | | w30.28 + 30.13 + | |
| 53.31 | | | 53.31 as a nucleic | |
| | | | acid e.g. DNA/MVA | |
| | | | vector and/or protein | |
| w78.15 + 100.B4 | | | | w78.15 + 100.B4 |
| | | | | as a nucleic acid |
| | | | | e.g. DNA/MVA |
| | | | | vector and/or protein |

A skilled artisan can readily determine the number and interval between boosts.

Table 2 shows a non-limiting example of a sequential immunization protocol using a swarm of HIV1 envelopes optionally in certain embodiments designed as trimers.

| Envelope | Prime | Boost(s) |
|---|---|---|
| 703010505.TF, | 703010505.TF | 703010505.TF, |
| 703010505.W4.03, | (optionally | 703010505.W4.03, |
| 703010505.W4.26, | 703010505.W4.03, | 703010505.W4.26, |
| 703010505.W14.21, | 703010505.W4.26) | 703010505.W14.21, |
| 703010505.W20.14, | as a nucleic acid | 703010505.W20.14, |
| 703010505.W30.28, | e.g. DNA/MVA | 703010505.W30.28, |
| 703010505.W30.13, | vector and/or protein | 703010505.W30.13, |
| 703010505.W53.31, | | 703010505.W53.31, |
| 703010505.W78.15, | | 703010505.W78.15, |
| 703010505.W100.B4. | | 703010505.W100.B4 |
| | | as a nucleic acid |
| | | e.g. DNA/MVA |
| | | vector and/or protein |

A skilled artisan can readily determine the number and interval between boosts

For a 20mer immunization regimen (envelopes (703010505.TF, 703010505.W4.03, 703010505.W4.26, 703010505.W14.3, 703010505.W14.8, 703010505.W14.21, 703010505.W20.7, 703010505.W20.26, 703010505.W20.9, 703010505.W20.14, 703010505.W30.28, 703010505.W30.12, 703010505.W30.19, 703010505.W30.13, 703010505.W53.19, 703010505.W53.13, 703010505.W53.31, 703010505.W78.1, 703010505.W78.15, 703010505.W100.B4), in a non-limiting embodiment, one can prime with CH505 TF+W4.03, then boost with a mixture of w4.26+14.21+20.14+14.3+14.8+20.7, then boost with a mixture of w 20.26+20.9+30.12+w30.28+30.13+53.31, then boost with a mixture of w78.15+100.B4+30.19+53.19+53.13+78.1. Other combinations of envelopes are contemplated for boosts.

Table 3 shows a non-limiting example of a sequential immunization protocol using a swarm of HIV1 envelopes optionally in certain embodiments designed as trimers

| Envelope | Prime | Boost(s) |
|---|---|---|
| 703010505.TF, | 703010505.TF, | 703010505.TF, |
| 703010505.W4.03, | (optionally | 703010505.W4.03, |
| 703010505.W4.26, | 703010505.W4.03, | 703010505.W4.26, |
| 703010505.W14.3, | 703010505.W4.26, | 703010505.W14.3, |
| 703010505.W14.8, | 703010505.W14.3, | 703010505.W14.8, |
| 703010505.W14.21, | 703010505.W14.8, | 703010505.W14.21, |
| 703010505.W20.7, | 703010505.W14.21), | 703010505.W20.7, |
| 703010505.W20.26, | as a nucleic acid | 703010505.W20.26, |
| 703010505.W20.9, | e.g. DNA/MVA | 703010505.W20.9, |
| 703010505.W20.14, | vector and/or protein | 703010505.W20.14, |
| 703010505.W30.28, | | 703010505.W30.28, |
| 703010505.W30.12, | | 703010505.W30.12, |
| 703010505.W30.19, | | 703010505.W30.19, |
| 703010505.W30.13, | | 703010505.W30.13, |
| 703010505.W53.19, | | 703010505.W53.19, |
| 703010505.W53.13, | | 703010505.W53.13, |
| 703010505.W53.31, | | 703010505.W53.31, |
| 703010505.W78.1, | | 703010505.W78.1, |
| 703010505.W78.15, | | 703010505.W78.15, |
| 703010505.W100.B4. | | 703010505.W100.B4. |
| | | as a nucleic acid |
| | | e.g. DNA/MVA |
| | | vector and/or protein |

A skilled artisan can readily determine the number and interval between boosts.

Table 4 shows a non-limiting example of a sequential immunization protocol using a swarm of HIV1 envelopes optionally in certain embodiments designed as trimers.

| Envelope | Prime | Boost(s) | Boost(s) | Boost(s) |
|---|---|---|---|---|
| CH505.M6 | CH505.M6 | | | |
| CH505.M11 | CH505.M11 | | | |
| | as a nucleic acid | | | |
| | e.g. DNA/MVA | | | |
| | vector and/or protein | | | |
| CH505w020.14 | | CH505w020.14 | | |
| CH505w030.28 | | CH505w030.28 | | |
| | | as a nucleic acid | | |
| | | e.g. DNA/MVA | | |
| | | vector and/or protein | | |
| CH505w078.15 | | | CH505w078.15 | |
| CH505w053.31 | | | CH505w053.31 | |
| CH505w030.21 | | | CH505w030.21as | |
| | | | a nucleic acid | |
| | | | e.g. DNA/MVA | |
| | | | vector and/or protein | |
| CH505w078.33 | | | | CH505w078.33 |
| CH505w053.16 | | | | CH505w053.16 |
| CH505w100.B6 | | | | CH505w100.B6 |
| | | | | as a nucleic acid |
| | | | | e.g. DNA/MVA |
| | | | | vector and/or protein |

A skilled artisan can readily determine the number and interval between boosts.

### Example 4

The immunization methods and compositions of Example 4 can further comprise an agent for transient immunomodulation of the host immune response during vaccination.

One embodiment of the invention would be to administer the “production 10” CH505 Envs in the following regimen with each immunization being followed with a regimen such as low dose CD25 antibody 0.5-5 mg 5-7 days after each immunization. Immunization 1 would be DNA gp120, gp145, or gp160+gp120 or gp140 Env protein of M11 and M6 Envs, Immunization 2 would be DNA+Env protein of week 20.14, w30.28 and w78.15 Envs. Immunization 3 would be DNA+Env protein of week 53.31, w30.21 and w78.33 Envs and immunization 4 would be w52.16+w100B6 DNA+Env protein in the designs of the first immunization ie gp120 gp145 or gp160 etc. the protein could be g-120 monomers, gp120 trimers or gp140 trimers.

### Example 5: Non-Human Primate Studies

NHP 79: CH505T/F gp120 envelope in GLA/SE. NHP 85: CH505T/F gp140 envelope in GLA/SE. This compares gp140 with gp120 induced antibodies.

NHP study of CH505T/F gp120 with GCN4 CH505 T/F in GLA/SE.

NHP study of CH505T/F gp120 with GCN4 CD40L CH505 T/F in GLA/SE.

NHP study of CH505T/F gp120 with GCN4 CD40L CH505 T/F in ALUM.

NHP study of CH505 T/F gp120 with GCN4 CD40L CH505 T/F=−HIS tag with liposomes in ALUM.

NHP study of M6 then rest of “production 10” (Table 4) gp120 in sequence gp120 GNC4 CD40L CH505 trimers with ALUM or GLA/SE (depends on antigenicity).

NHP study of M6 then rest of production 10 (Table 4) gp120 in sequence gp120 GNC4 CD40L CH505 trimers in ALUM or GLA/SE (depends on antigenicity), with a dose of chloloquine orally each day 10 days before each immunization and then a dose of CD25 Ab 5 days after each immunization.

### Example 6: A New Pathway for Central B-Cell Tolerance: Interaction of AID, MyD88, and BCR

Example 6 shows that BCR and MyD88 signals synergize to increase AID expression in autoreactive immature and transitional B cells and to mediate their loss by apoptosis.

Expression of activation-induced cytidine deaminase (AID) in immature and transitional B cells in mice and humans is genetically linked to the first tolerance checkpoint^{1,2}. In the absence of AID, autoreactive immature/transitional-1 (T1) B cells are inefficiently purged and exhibit increased resistance to receptor-induced apoptosis¹. These substantial effects are surprising given that AID expression in the immature/T1 B-cell pools is only 3% of that in germinal center (GC) B cells^{1,3}. It is now shown that B-cell antigen receptor (BCR) and Toll-like receptor (TLR) signaling synergize to elicit high levels of AID expression in immature/T1 B cells. This synergy is restricted to intracellular TLR ligands, requires both activation of phospholipase-D and intracellular acidification, and acts to ensure self-tolerance through a process that requires Myd88. Consistent with the requirement of AID and MyD88 for central B-cell tolerance and the role for intracellular acidification in activating intracellular TLR signaling and in the synergistic AID increase in immature/T1 B cells, mice treated with chloroquine exhibited relaxed central B-cell tolerance. These findings identify a novel mechanism for central B-cell tolerance and resolve several problematic weaknesses of current models for the first tolerance checkpoint. It is suggested that this intracellular acidification/MyD88/AID-mediated pathway may be the primary mechanism for central B-cell tolerance by deletion.

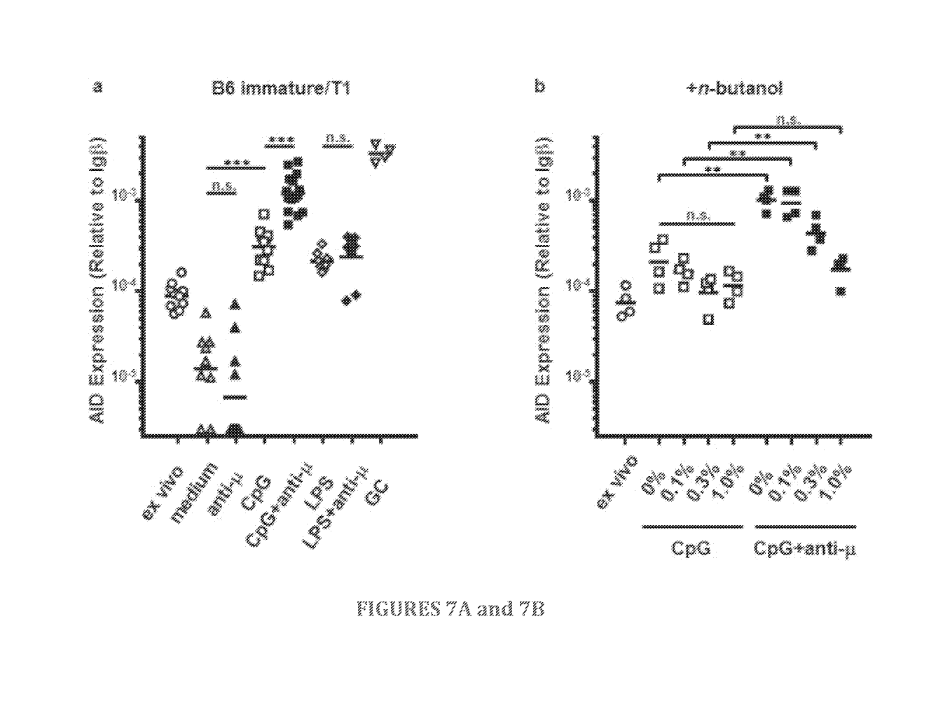

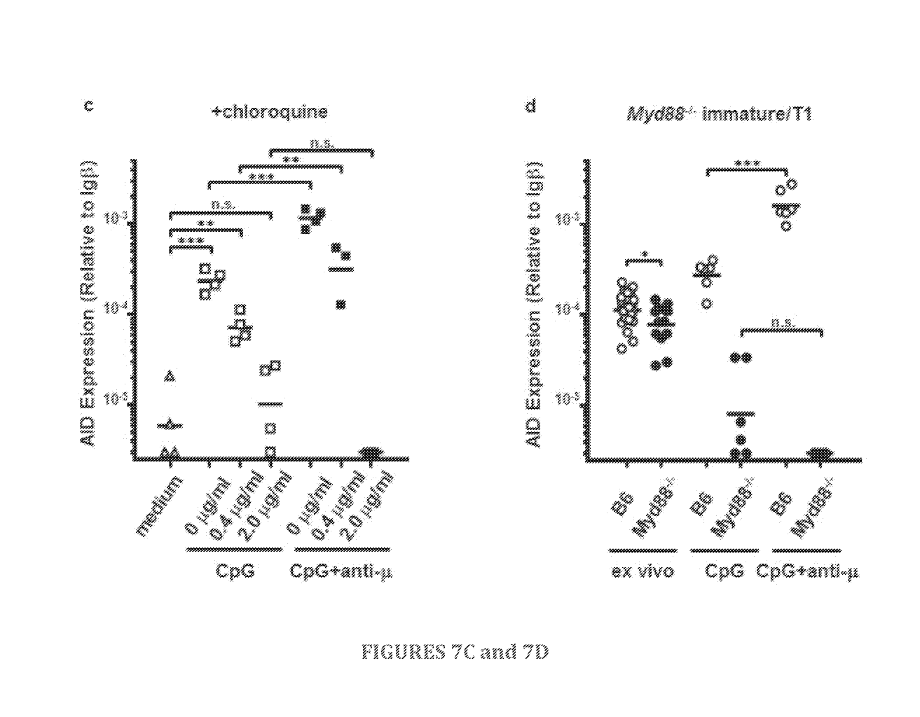

BCR- and TLR signaling synergistically activate mature, anti-DNA autoreactive B cells⁴, suggesting co-operative roles for these signaling in the regulation of B cells. To determine whether these signaling pathways elicit high levels of AID expression in immature/T1 B cells, immature/T1 B cells were sorted from bone marrow of B6 mice and these cells were stimulated with F(ab′)₂ anti-IgM antibody (anti-μ), CpG, LPS, or combination of these stimuli in vitro, and the AID message levels were quantified. As expected⁵, AID expression in immature/T1 B cells was elevated by stimulations with TLR ligands, CpG and LPS (FIG. 7a). Interestingly, co-activation with CpG and anti-μ further elevated AID expression in immature/T1 B cells, AID levels approaching to approximately 35% of GC B cells (FIG. 7a). It is plausible that BCR- and TLR signaling synergistically, rather than additively, increased AID expression in immature/T1 B cells, as anti-μ alone did not increase AID expression (FIG. 7a). This synergy was not seen in immature/T1 B cells co-activated with LPS and anti-μ (FIG. 7a), and was delayed in splenic mature B cells (Extended data FIG. 7)⁶. These results suggest unique, cooperative roles for BCR- and intracellular TLR signaling in the regulation of AID expression in immature/T1 B cells.

To explore mechanistic insight of this synergy, specific inhibitors were used to block respective intracellular events and signaling pathways. Upon anti-μ stimulation, surface BCR are internalized and located in the phagosome-like compartment′. Concomitantly, intracellular TLR, such as TLR7 and TLR9, are recruited to the same compartment in an activation of phospholipase D-dependent manner^{7,8}. It is hypothesized that the co-localization of BCR and intracellular TLR, and subsequent unique signaling^{7,8} might be involved in the synergy. To test this possibility, activation of phospholipase D was blocked with normal (n)-butanol and assessed AID expression by immature/T1 B cells in vitro (FIG. 7b). Addition of n-butanol did not significantly change the levels of AID expression in immature/T1 B cells stimulated with CpG, suggesting that activation of phospholipase-D is not required for CpG-induced AID expression by immature/T1 B cells (FIG. 7b). By contrast, the synergistic effects of CpG and anti-μ co-stimulation on AID expression in immature/T1 B cells were suppressed by n-butanol in a dose-dependent manner and completely abrogated at 1.0% (FIG. 7b). These results suggest that the trafficking of TLR9 to the phagosome-like compartments is required for the synergistic increase of AID expression in immature/T1 B cells.

Both intracellular acidification and the adaptor protein MyD88 are essential for TLR9-mediated signaling. To determine if intracellular acidification is also required for the synergy, immature/T1 B cells were stimulated with CpG or CpG+anti-μ in the presence or absence of chloroquine. It was found that AID expression in immature/T1 B cells elevated by CpG or CpG+anti-μ stimulation was abrogated in the presence of 2.0 μg/ml of chloroquine (FIG. 7c), indicating essential role for intracellular acidification in the AID expression by immature/T1 B cells. To determine whether AID expression in immature/T1 B cells is also dependent on MyD88, Myd88^{+/+} and Myd88^{-/-} immature B cells were compared for AID expression. Although moderate, AID expression in freshly isolated Myd88^{-/-} immature/T1 B cells was significantly lower (P<0.05) than that in B6 counterparts⁹ (FIG. 7d). More dramatically, but in agreement with the role for MyD88 in TLR9 signaling, AID expression in Myd88^{-/-} immature/T1 B cells were not elevated by CpG or CpG+anti-μ stimulations (FIG. 7d). Thus, the synergistic increase of AID expression in immature/T1 B cells induced by CpG+anti-μ co-stimulation is dependent on both intracellular acidification and Myd88.

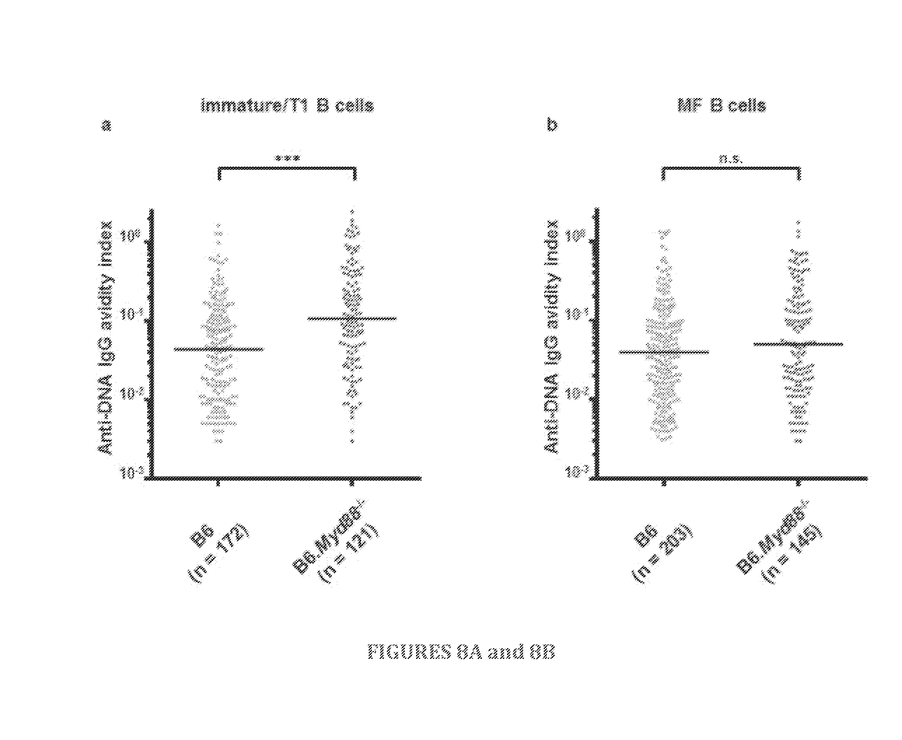

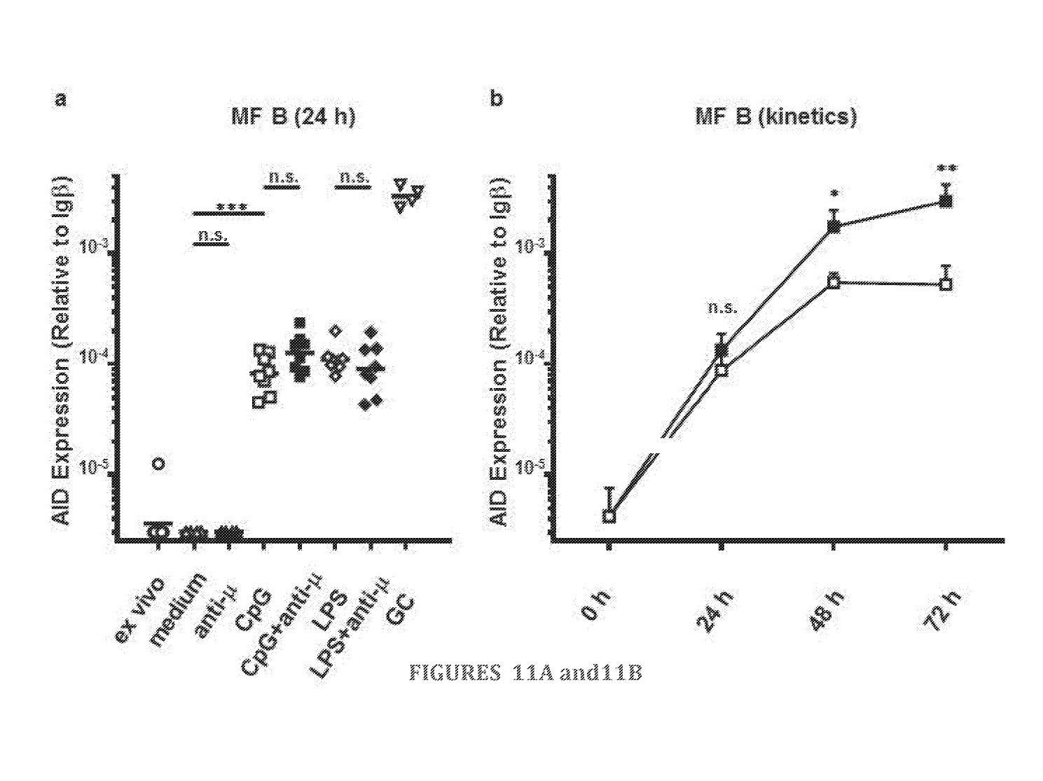

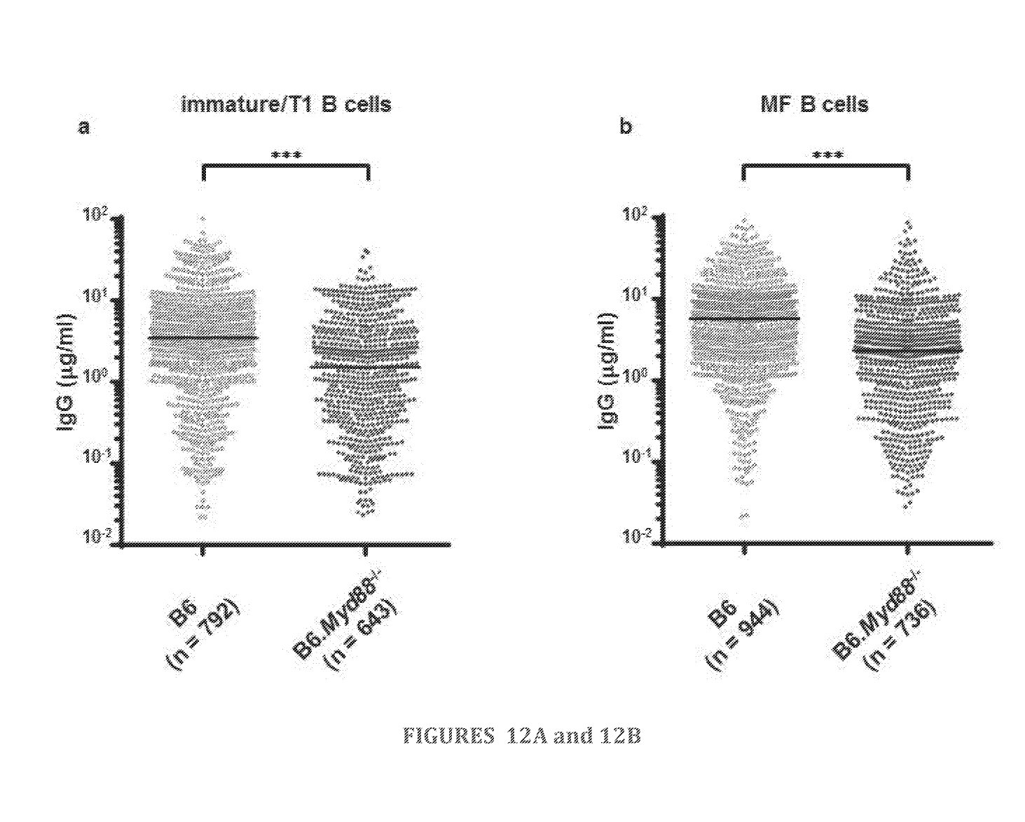

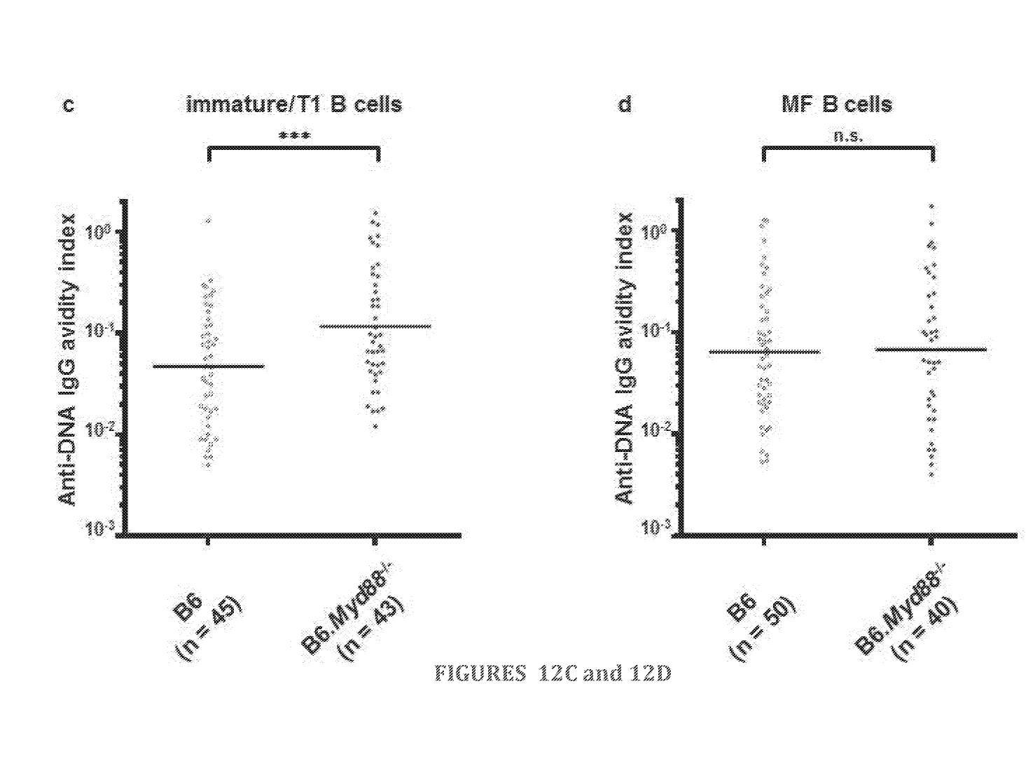

Given that AID expression in immature/T1 B cells is required for central B-cell tolerance^{1,2}, lack of the synergistic AID increase in immature/T1 B cells could also result in defective central B-cell tolerance in Myd88^{-/-} mice. Indeed, the first tolerance checkpoint is mitigated in a MyD88-deficient patient¹⁰. To determine if MyD88 plays any role in central B-cell tolerance, repertoires of immature/T1 B cells between wild type and Myd88^{-/-} mice were compared by measuring frequency and avidity of anti-DNA IgG obtained from single B-cell cultures; single immature/T1 B cells and MF B cells from B6 and Myd88^{-/-} mice were sorted, and CD154-/BAFF-/IL-21-expressing feeder cells were used to culture on, which induce proliferation and differentiation into IgG-secreting cells. The average 1.2-4.5 μg/ml IgG in culture supernatants (Extended data FIG. 12a) was obtained. Although frequencies of DNA-binding IgG did not significantly change among B-cell subsets (19-22%), anti-DNA IgG from Myd88^{-/-} immature/T1 B-cell cultures more avidly (DNA avidity index, 0.108 vs 0.044; P<0.001) bound to DNA than those from wild type immature/T1 B-cell cultures (FIG. 8a). Our results strongly suggest that Myd88 is required for purging autoreactive immature/T1 B cells. In contrast, B cells that avidly bound to DNA were not retained in splenic MF B-cell compartments (FIG. 8b), suggesting that peripheral tolerance checkpoint is normal in Myd88^{-/-} mice or that autoreactive B cells are retained in other B-cell compartments of spleen, such as IgM^{lo/-}IgD⁺ anergic B cells.

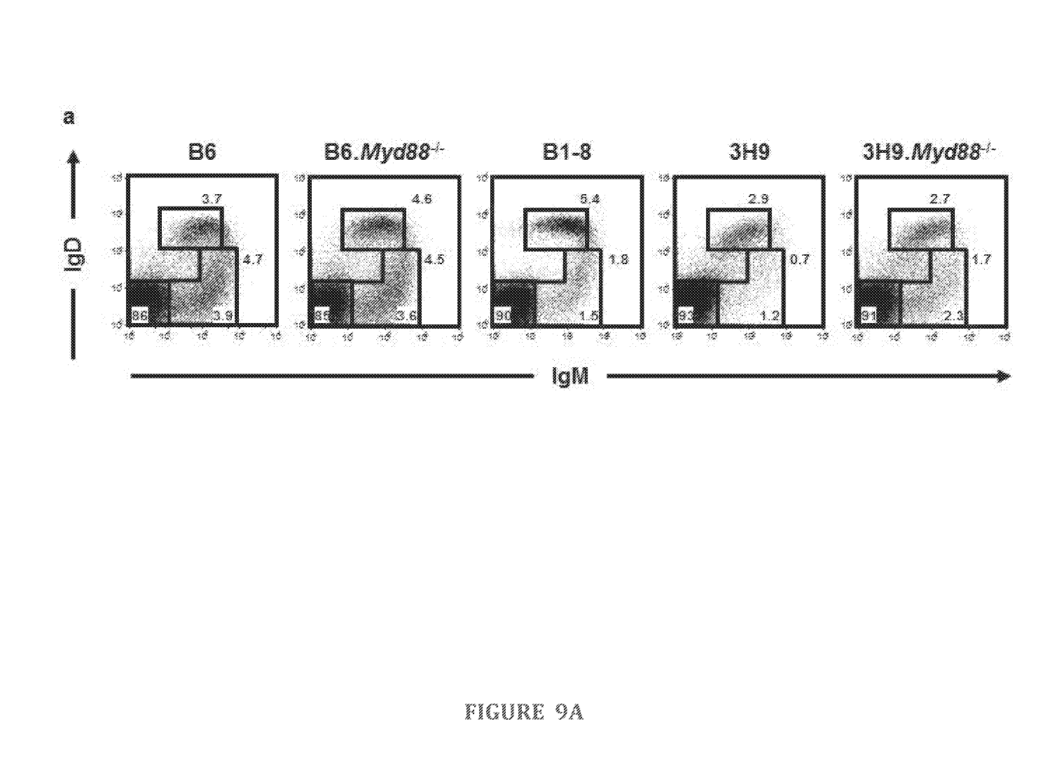

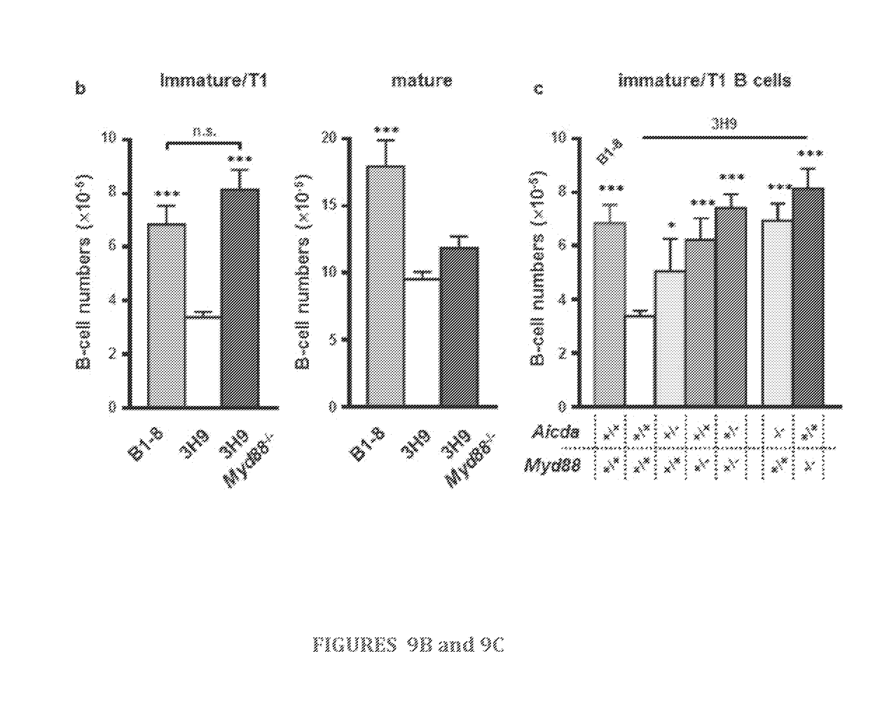

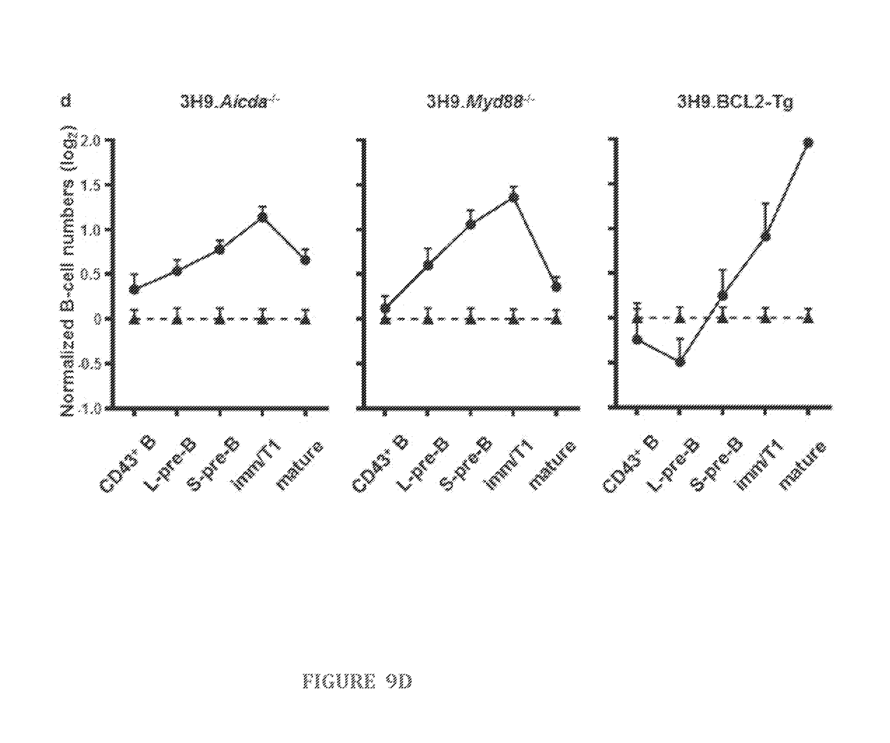

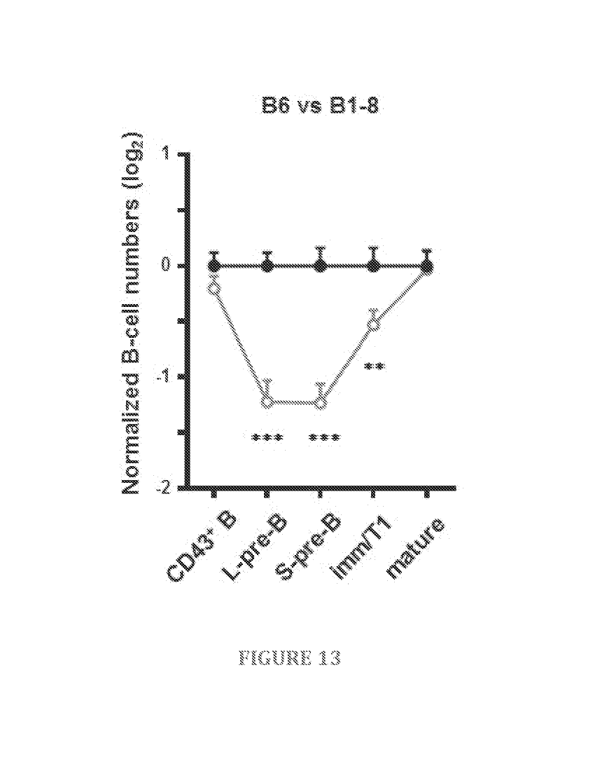

To assess effects of Myd88 on development of DNA-reactive B cells, Myd88^{-/-} mice homozygous were generated for the autoreactive 3H9 VDJ knock-in allele¹¹ and B-cell development in the bone marrow of these animals was examined by flow cytometry¹. As B-cell development in mice homozygous for the “innocent” VDJ allele (B1-8 mice) were substantially different from that in wild-type mice (FIG. 13), B1-8 mice were used as non-autoreactive controls for 3H9 mice. Consistent with the tolerizing deletion of autoreactive 3H9 B cells′, the numbers of immature/T1 B cells were significantly reduced (P<0.001) in 3H9 mice (FIG. 9). In contrast, immature/T1 B-cell numbers were substantially increased (P<0.001) in 3H9.Myd88^{-/-} mice to the levels comparable to those in B1-8 mice (FIG. 9a, b). Given that the loss of bone marrow immature/T1 B cells in 3H9 mice were also mitigated on an Eμ-BCL2 transgenic background, it is proposed that BCR- and TLR-Myd88 pathways play a primary role in purging immature/T1 B cells by inducing apoptosis which can be protected by BCL2 in autoimmune 3H9 knock-in mice.

The analysis of 3H9 mice heterozygous for Aicda, Myd88, or both (Aicda^{+/-}Myd88^{+/-}) revealed inverse correlation between gene dosage and the numbers of immature/T1 B cells in the bone marrow (FIG. 9c). The relative increase of 3H9 immature/T1 B-cell numbers in the bone marrow of 3H9.Aicda^{+/-} and 3H9.Myd88^{+/-} mice were 60% and 48%, respectively, of their homozygous knockouts (FIG. 9c). Interestingly, 3H9 immature/T1 B-cell numbers in 3H9.Aicda^{+/-} Myd88^{+/-} double heterozygous mice were comparable (P≧0.426) to those in 3H9.Aicda^{-/-} and 3H9.Myd88^{-/-} mice (FIG. 9c). It is concluded that Aicda and Myd88 genes cooperatively establish central tolerance in B cells. Consistent with this conclusion, Aicda and Myd88 genes exhibited mirroring effects on B-cell development in the bone marrow of 3H9 mice (FIG. 9d).

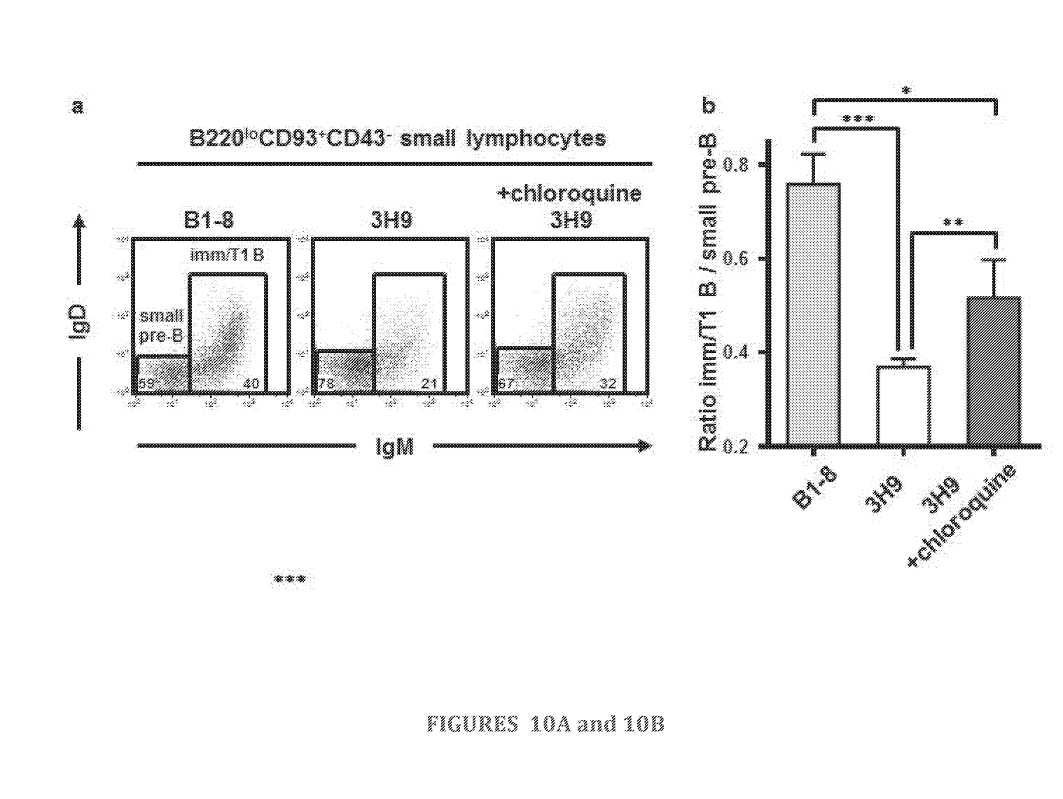

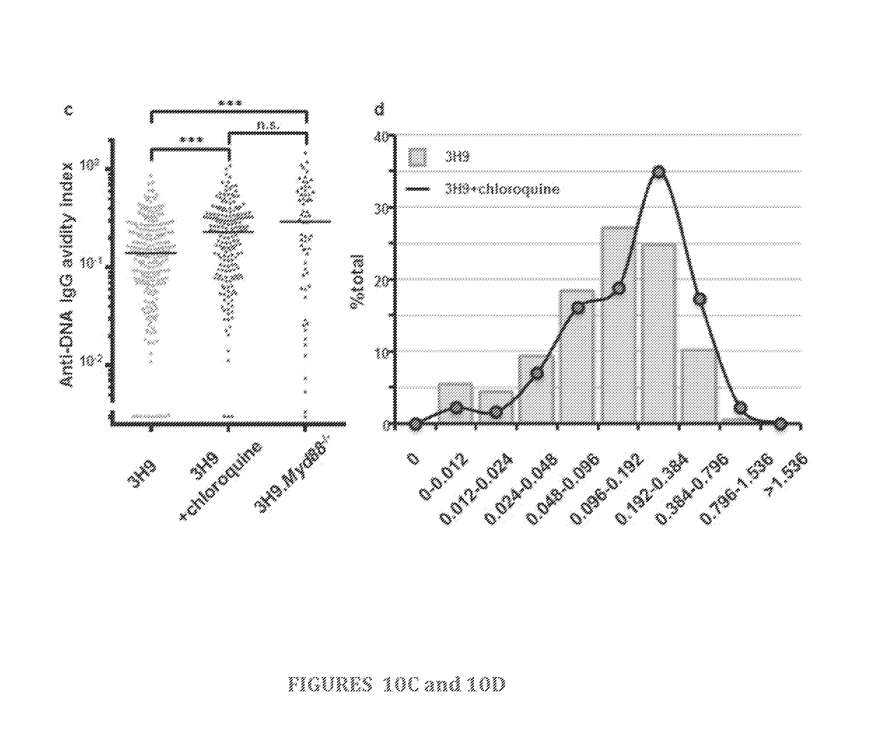

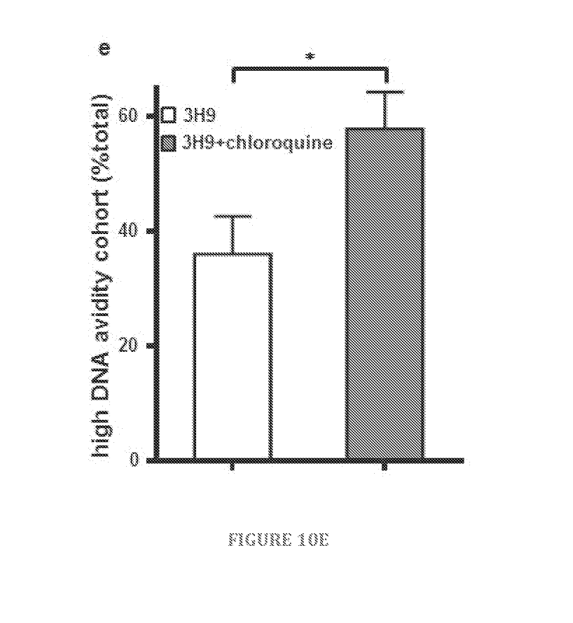

To determine whether intracellular acidification plays a role in central B-cell tolerance, 3H9 mice were treated with multiple injections of chloroquine for up to 8 days, and B-cell development in the bone marrow of these animals was assessed. The numbers of large- and small pre-B cells decreased in 3H9 mice treated with chloroquine. In contrast, the chloroquine treatment augmented the transition of small pre-B to immature/T1 B cells and/or the retention of immature/T1 B cells in bone marrow of 3H9 mice, as the ratio of immature/T1 B cells over small pre-B cells significantly increased in chloroquine-treated 3H9 mice (FIG. 10a, b). Importantly, this enhanced generation of immature/T1 B cells was associated with accumulation of immature/T1 B cells, which avidly bound to DNA (FIG. 10c-e): the average anti-DNA avidity indices of individual immature/T1 B cells was significantly higher in chloroquine-treated 3H9 mice (FIG. 10c), the distribution of anti-DNA avidity indices shifted toward higher avidity cohorts (FIG. 10d) and the frequency of immature/T1 B cells with higher DNA avidity (DNA avidity index 0.178, average DNA avidity index of control 3H9 immature/T1 B cells) increased in chloroquine-treated 3H9 mice (FIG. 10e). These results strongly suggest novel and unanticipated roles for intracellular acidification in central B-cell tolerance.

Tolerance mechanisms negatively impact on B-cell development in mice homozygous for the anti-HIV-1 2F5 heavy- and light chain rearrangements (2F5 mice). It is hypothesized that B-cell tolerance mediated by intracellular acidification also play roles in the suppression of B cells in 2F5 mice. To test this, 2F5 mice were treated with chloroquine or PBS and B-cell development in these mice was compared. The chloroquine injections relaxed the severe suppression of B-cell development in 2F5 mice.

Example 6 shows that chloroquine treatment relaxed B-cell tolerance and altered B-cell repertoires in mice. It is a useful strategy to develop effective vaccine against pathogens, such as HIV-1.

Methods

Mice and Immunizations

Female C57BL/6 (B6), and congenic AID-deficient mice (B6(B6CB)-Aica^{tm1Hon}; Aicda^{-/-})¹⁴, MyD88-deficient mice (B6.129-Myd8^{tm1Aki}; Myd88^{-/-})¹⁵, 3H9 heavy chain knock-in mice (B6.129P2(Cg)-Igh-J^{tm1(3H9-VDJ)Mwg})¹¹, 3H9.Aicda^{-/- mice (}3H9×Aicda^{-/-})¹, 3H9.Myd88^{-/-} mice, and 3H9.Bcl2-Tg mice (3H9×B6.Cg-Tg(BCL2)22Wehi/J¹⁸) (all B6 background) were bred and maintained under specific pathogen-free conditions at the Duke University Animal Care Facility. Mice used in experiments were 7-12 weeks of age. All experiments involving animals were approved by the Duke University Institutional Animal Care and Use Committee.

In some experiments, 3H9 mice were injected i.p. with 100 μl PBS with or without 1.2 mg of chloroquine daily from day 0 to day 4, and then twice a day from day 4 to day 7 or day 8.

Flow Cytometry and Definition of Hematopoietic Populations

Specific B-lineage developmental compartments were identified as described¹: CD43⁺ B (pre-pro-B and pro-B; B220^{low}CD93⁺IgM⁻IgD⁻CD43⁺), large pre-B (B220^{low}CD93⁺IgM⁻ IgD⁻CD43⁻FSC^{high}), small pre-B (B220^{low}CD93⁺IgM⁻IgD⁻CD43⁻FSC^{low}), immature/T1 B (B220^{low}CD93⁺IgM⁺IgD^{+/-}CD21⁻CD23⁻) and mature B (B220^{hi}CD93⁻) cells in bone marrow, and splenic MF B (B220^{hi}CD93⁻IgM^{int}IgD^{hi}CD21^{int}CD23^{hi}) cells of naïve mice, and germinal center B (GL-7⁺B220^{high}Fas⁺IgD⁻) cells in spleen of NP-CGG/alum immunized mice. Specific developmental compartments were designated for B6, and then identical gatings were used for all samples. Cells that take up propidium iodide were excluded from our analyses. Labeled cells were analyzed/sorted in a FACS Canto (BD Bioscience) or FACS Vantage with DIVA option (BD Bioscience). Flow cytometric data were analyzed with FlowJo software (Treestar Inc.).

Cell Cultures

Sorted bone marrow immature/T1 B cells and splenic MF B cells (2.5×10⁴ cells/well) were cultured in IMDM (Invitrogen) containing 10% HyClone FBS (Thermo scientific), 2-mercaptoethanol (5.5×10⁻⁵ M), penicillin (100 units/ml), streptomycin (100 μg/ml; all Invitrogen) and recombinant BAFF (250 ng/ml; R & D systems), in the presence or absence of F(ab′)₂ fragment of anti-IgM (anti-0 Ab (10 μg/ml; Jackson Immunoresearch), CpG (ODN1826, 0.5 and 5 μg/ml; InvivoGen), LPS (0127:B8, 0.5 and 5 μg/ml; Sigma) or combinations of these stimuli. In some cultures, n-butanol (0.1, 0.3, and 1.0%; Sigma) or chloroquine (0.4 and 2.0 μg/ml; Sigma) was also added. Twenty-four hours after culture, B cells were harvested in TRIzol-LS reagent for AID mRNA quantification.

Single immature/T1 B cells and MF B cells from unimmunized B6 and Myd88^{-/-} mice, and control- and chloroquine-treated 3H9 mice were expanded in the presence of NB-21.2D9 feeder cells (N-cultures). Briefly, single immature/T1 B cells and MF B cells were directly sorted into each well of 96-well plates and cultured in the presence of exogenous recombinant IL-4 (2 ng/ml, Peprotech) and CD154-/BAFF-/IL-21-expressing NB-21.2D9 cells in RPMI 1640 (Invitrogen) supplemented with 10% HyClone FBS (Thermo scientific), 2-mercaptoethanol (5.5×10⁻⁵ M), penicillin (100 units/ml), streptomycin (100 μg/ml), HEPES (10 mM), sodiumpyruvate (1 mM), and MEM nonessential amino acid (1×; all Invitrogen). Two thirds of culture media were replaced with fresh media daily from day 2 to day 8. On days 9-10, culture supernatants were harvested for ELISA determinations.

Quantitative RT-PCR and Quantification of AID Expression

Expression of AID mRNA was determined by a quantitative RT-PCR¹. Briefly, sorted immature/T1 and mature follicular B cells were lysed in TRIzol LS before and after cultures. Total RNA was prepared from these cells using standard phenol/chloroform extraction method, and then cDNA was prepared using SuperScript III reverse transcriptase (Invitrogen) with oligo(dT)₂₀ primers (Invitrogen). One-twentieth volume of cDNA samples were amplified in a primary PCR using Ramp-Taq DNA polymerase (Denville Scientific) with AID118 and AID119 primers¹⁴. Primary PCR condition: initial incubation of 95° C. for 7 min followed by 15 cycles of amplification steps (95° C. for 30 s, 58° C. for 20 sec, and 72° C. for 45 sec.). Primary PCR products were then subjected to quantitative PCR using SYBR Green core reagents and AIDF2/AIDR2 primers⁵.

ELISA Determinations

Concentrations of total IgG in culture supernatants were determined by standard ELISA. Briefly, 96-well ELISA plates (Corning) were coated with anti-mouse Igκ Ab and anti-mouse Igλ Ab (2 μg/ml each; Southern Biotech) in carbonate buffer for overnight. After washing, plates were blocked with PBS containing 0.5% BSA for 1 h. Diluted samples (at 1:100 and 1:1,000 dilutions in PBS containing 0.5% BSA and 0.1% Tween-20) and serially diluted standard anti-DNA mAb (HYB331-01; Abcam) were then applied to the plates and incubated for overnight. After washing, HRP-conjugated anti-mouse IgG (Southern Biotech) was added to the plates and incubated for 2 h. The HRP-activity was visualized with TMB substrate reagents (Biolegend) and OD₄₅₀-OD₆₂₀ was measured by spectrophotometer (Bio-Rad).

Anti-DNA IgG was measured by ELISA¹⁹. Briefly, ELISA plates were coated with phenol/chloroform-purified calf thymus DNA (Sigma) in 1×SSC (10 μg/ml) and dried up at 37° C. for overnight. After blocking with hypotonic buffer (1.5×10⁻² M NaCl, 4.3×10⁻⁴ M Na₂HPO₄, and 1.9×10⁻³ M NaH₂PO₄) containing 3% FBS and 0.5% BSA for 1 h, samples (at 1:10 dilutions in hypotonic buffer containing 0.5% BSA and 0.1% Tween-20) and serially diluted standard anti-DNA mAb were applied to the plates and incubated for overnight. Bound IgG was detected by HRP-conjugated anti-mouse IgG and TMB substrates as described above. The cut-off OD₄₅₀-OD₆₂₀ values for total IgG and anti-DNA IgG were set at the point representing six-standard deviations above the mean OD₄₅₀-OD₆₂₀ values for supernatants from mock-treated, B-cell negative culture supernatant samples.

DNA avidity index, [anti-DNA IgG]/[total IgG], represents proportion of DNA-binding IgG to total IgG in reference to the anti-DNA mAb. To compare DNA avidity indices of individual immature/T1 B cells from control- and chloroquine-treated 3H9 mice, culture supernatant samples that contain total IgG (range: 1-3 μg/ml) were analyzed. Hapten-specific mAb, H33Lγ1²⁰ was used as a negative control for DNA binding and (DNA avidity index of sample)<(DNA avidity index of H33Lγ1=0.003) was considered as negative in the assays.

Statistical Analyses of Data

Statistical significance (P<0.05) was determined by two-tailed Student's t test and Mann-Whitney's U test.

### REFERENCES FOR EXAMPLE 6

1 Kuraoka, M. et al. Activation-induced cytidine deaminase mediates central tolerance in B cells. Proc Natl Acad Sci USA (2011).
2 Meyers, G. et al. Activation-induced cytidine deaminase (AID) is required for B-cell tolerance in humans. Proc Natl Acad Sci USA (2011).
3 Kuraoka, M. et al. Activation-induced cytidine deaminase expression and activity in the absence of germinal centers: insights into hyper-IgM syndrome. J Immunol 183, 3237-3248 (2009).
4 Leadbetter, E. A. et al. Chromatin-IgG complexes activate B cells by dual engagement of IgM and Toll-like receptors. Nature 416, 603-607 (2002).
5 Ueda, Y., Liao, D., Yang, K., Patel, A. & Kelsoe, G. T-independent activation-induced cytidine deaminase expression, class-switch recombination, and antibody production by immature/transitional 1 B cells. J Immunol 178, 3593-3601 (2007).
6 Pone, E. J. et al. BCR-signalling synergizes with TLR-signalling for induction of AID and immunoglobulin class-switching through the non-canonical NF-kappaB pathway. Nat Commun 3, 767 (2012).
7 Chaturvedi, A., Dorward, D. & Pierce, S. K. The B cell receptor governs the subcellular location of Toll-like receptor 9 leading to hyperresponses to DNA-containing antigens. Immunity 28, 799-809 (2008).
8 Chaturvedi, A., Martz, R., Dorward, D., Waisberg, M. & Pierce, S. K. Endocytosed BCRs sequentially regulate MAPK and Akt signaling pathways from intracellular compartments. Nat Immunol 12, 1119-1126 (2011).
9 Han, J. H. et al. Class switch recombination and somatic hypermutation in early mouse B cells are mediated by B cell and Toll-like receptors. Immunity 27, 64-75 (2007).
10 Isnardi, I. et al. IRAK-4- and MyD88-dependent pathways are essential for the removal of developing autoreactive B cells in humans. Immunity 29, 746-757 (2008).
11 Chen, C., Nagy, Z., Prak, E. L. & Weigert, M. Immunoglobulin heavy chain gene replacement: a mechanism of receptor editing. Immunity 3, 747-755 (1995).
12 Chen, C. et al. The site and stage of anti-DNA B-cell deletion. Nature 373, 252-255 (1995).
13 Di Noia, J. M. & Neuberger, M. S. Molecular mechanisms of antibody somatic hypermutation. Annu Rev Biochem 76, 1-22 (2007).
14 Muramatsu, M. et al. Class switch recombination and hypermutation require activation-induced cytidine deaminase (AID), a potential RNA editing enzyme. Cell 102, 553-563 (2000).
15 Adachi, O. et al. Targeted disruption of the MyD88 gene results in loss of IL-1- and IL-18-mediated function. Immunity 9, 143-150 (1998).
16 Nilsen, H. et al. Uracil-DNA glycosylase (UNG)-deficient mice reveal a primary role of the enzyme during DNA replication. Mol Cell 5, 1059-1065 (2000).
17 Reitmair, A. H. et al. MSH2 deficient mice are viable and susceptible to lymphoid tumours. Nat Genet 11, 64-70 (1995).
18 Strasser, A. et al. Enforced BCL2 expression in B-lymphoid cells prolongs antibody responses and elicits autoimmune disease. Proc Natl Acad Sci USA 88, 8661-8665 (1991).
19 Chen, Z., Koralov, S. B. & Kelsoe, G. Complement C4 inhibits systemic autoimmunity through a mechanism independent of complement receptors CR1 and CR2. J Exp Med 192, 1339-1352 (2000).
20 Dal Porto, J. M., Haberman, A. M., Shlomchik, M. J. & Kelsoe, G. Antigen drives very low affinity B cells to become plasmacytes and enter germinal centers. J Immunol 161, 5373-5381 (1998).

### Example 7: Oral Chloroquine Limits B-Cell Tolerance In Vivo

Chloroquine blocked synergistic AID up-regulation in vitro (Example 6). FIGS. 14-20 shows experiments and results that assessed whether chloroquine relaxes central tolerance in vivo.

FIG. 20 shows that injections of chloroquine release 2F5 dKI B cells from tolerizing deletion. As a result, 2F5 dKI B cells that are normally removed by tolerance are available in chloroquine-treated mice. These B cells respond to the subsequent immunization with MPER liposomes and elicit stronger germinal center (GC) responses. A single injection of anti-CD25 Abs suppresses Treg cells, leading to the prolonged B-cell recruitment into GCs. Prolonged B-cell recruitment allow rare B cells (such as MPER-specific B cells) to engage GC reactions.

Whether administrations of chloroquine and/or anti-CD25 Abs enhance GC responses in 2F5 dKI mice is being tested. FIG. 20B shows that: (1) Absolute cell numbers of splenic transitional-1 (T1) and T2 increased after consecutive injections of 2F5 dKI mice with chloroquine and MPER liposome (14 days after the last chloroquine injections—that is 12 days after MPER liposome immunization); (2) Single injection of anti-CD25 Ab (PC61) decreased absolute cell numbers of B-cell subsets in spleen (compare open and filled bars in PBS group), but those effects were not seen in mice received chloroquine (compare open and filled bars in chloroquine group); (3) GC responses observed in controls (PBS+control IgG) were abolished by chloroquine injections or by anti-CD25 Ab injection. This suggests the importance of timing of both the immunization after chloroquine injections and the anti-CD25 injections after immunization to optimally elicit primary GC responses.

The contents of all documents and other information sources cited herein are herein incorporated by reference in their entirety.

What is claimed is:

1. A method of inducing an immune response in a subject in need thereof comprising administering a composition comprising an HIV-1 immunogen, or a combination of several HIV-1 immunogens, and a first immunomodulatory agent in an amount sufficient to induce an immune response.

2. The method of claim 1, wherein the first immunomodulatory agent is administered in at time and in an amount sufficient for transient modulation of the subject's immune response so as to induce an immune response which comprises broad neutralizing antibodies against HIV-1 envelope

3. The method of claim 1, wherein the HIV-1 immunogen is HIV-1 envelope, a fragment thereof, or a peptide derived from HIV-1 envelope.

4. The method of claim 1, wherein the immune response is a humoral immune response.

5. The method of claim 2, wherein the modulation includes PD-1 blockade; T regulatory cell depletion; CD40L hyperstimulation; soluble antigen administration, or a combination thereof.

6. The method of claim 4, wherein the humoral immune response comprises the induction of a CD4bs bNAb, a V3 bNAb, V1V2 bNAb, gp41 bNAb, or a combination of antibodies.

7. The method of claim 1, wherein the agent is chloroquine (CQ), PTP1B Inhibitor—CAS 765317-72-4, MSI 1436, clodronate or any other bisphosphonate, a Foxo1 inhibitor, Gleevac, anti-CD25 antibody, anti-CCR4 Ab, an agent which binds to a B cell receptor for a dominant HIV-1 envelope epitope, or any combination thereof.

8. The method of claim 1, further comprising administering a second immunomodulatory agent, wherein the second and first immunomodulatory agents are different.

9. The method of claim 8, wherein the first agent is administered before immunization with the HIV-1 immunogen.

10. The method of claim 1, wherein the HIV-1 immunogen is comprised in a composition which is administered as a nucleic acid, a protein or any combination thereof.

11. The method of claim 10, wherein the nucleic acid encoding the HIV-1 immunogen is operably linked to a promoter inserted in an expression vector.

12. The method of claim 10, wherein the protein is recombinant.

13. The method of claim 10, wherein the HIV-1 immunogen is comprised in a composition which is administered as a prime, a boost, or both.

14. The method of claim 10, wherein the HIV-1 immunogen is comprised in a composition which is administered as a multiple boosts.

15. The method of claim 10, wherein the HIV-1 immunogen is comprised in a composition with an adjuvant.


SEQUENCE LISTING (108 sequences)

SEQ ID NO: 1 - protein, 28 aa, Human immunodeficiency virus
NEQELLELDKWASLWNWFNITNWLWYIK

SEQ ID NO: 2 - protein, 28 aa, Human immunodeficiency virus
NEQDLLALDKWASLWNWFDISNWLWYIK

SEQ ID NO: 3 - protein, 28 aa, Human immunodeficiency virus
NEKDLLALDSWKNLWNWFSITKWLWYIK

SEQ ID NO: 4 - protein, 28 aa, Human immunodeficiency virus
NEQELLALDKWNNLWSWFDITNWLWYIR

SEQ ID NO: 5 - protein, 45 aa, Artificial Sequence
  Description of Artificial Sequence Synthetic polypeptide
NEQELLELDKWASLWNWFNITNWLWYIKYKRWIILGLNKIVRMYS

SEQ ID NO: 6 - protein, 45 aa, Artificial Sequence
  Description of Artificial Sequence Synthetic polypeptide
NEQDLLALDKWASLWNWFDISNWLWYIKYKRWIILGLNKIVRMYS

SEQ ID NO: 7 - protein, 45 aa, Artificial Sequence
  Description of Artificial Sequence Synthetic polypeptide
NEKDLLALDSWKNLWNWFSITKWLWYIKYKRWIILGLNKIVRMYS

SEQ ID NO: 8 - protein, 45 aa, Artificial Sequence
  Description of Artificial Sequence Synthetic polypeptide
NEQELLALDKWNNLWSWFDITNWLWYIRYKRWIILGLNKIVRMYS

SEQ ID NO: 9 - protein, 17 aa, Artificial Sequence
  Description of Artificial Sequence Synthetic peptide
YKRWIILGLNKIVRMYS

SEQ ID NO: 10 - protein, 33 aa, Artificial Sequence
  Description of Artificial Sequence Synthetic polypeptide
RMKQIEDKIEEILSKIYHIENEIARIKKLIGER

SEQ ID NO: 11 - protein, 6 aa, Artificial Sequence
  Description of Artificial Sequence Synthetic peptide
GSGSGS

SEQ ID NO: 12 - protein, 11 aa, Artificial Sequence
  Description of Artificial Sequence Synthetic peptide
GGSGGSGGSGG

SEQ ID NO: 13 - protein, 28 aa, Artificial Sequence
  Description of Artificial Sequence Synthetic peptide
NEKDLLALDSWKNLWNWFDITKWLWYIK

SEQ ID NO: 14 - protein, 169 aa, Artificial Sequence
  Description of Artificial Sequence Synthetic polypeptide
MGSSHHHHHHSSGLVPRGSHMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 15 - DNA, 1507 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgcgaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatcaacgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaaggtcaccgaattcgggacccggatcc

SEQ ID NO: 16 - DNA, 1507 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatcaacgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaaggtgaccgaattcaggtcccggatcc

SEQ ID NO: 17 - DNA, 1507 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaacaactccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgcgaagaccatcatcgtgcacctgaacgagtccgtgaagatcgagtgcacccgcccgaacaacaagacgcgcacgtccatccggatcggccctggccaggccttctacgccaccggccaggtgatcggcgacatccgcaaggcgtactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgctccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaagggacccgaattcggtcaccggatcc

SEQ ID NO: 18 - DNA, 1507 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagatgaccccgctgtgcgtgaccctgaactgcaccaacgccaccgcgatcaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgacaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgcactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaagggtcctgaattcggttaccggatcc

SEQ ID NO: 19 - DNA, 1519 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgcgacgacgaacgccaccgcgtccaactcgtccatcatcgaggagatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacacggcgaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccggtgccgcatcaagcagatcatcaacatgtggcaggaggtgggccgcgccatgtacgcaccgcccatcgccggcaacatcacctgcatctccaacatcaccggcctcctgctgacccgcgacggcggcgagaacaacacggagaccttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaagggtcccgaattcggttaccggatcc

SEQ ID NO: 20 - DNA, 1547 bp, Artificial Sequence
  Description of Artificial Sequence Synthetic polynucleotide
ggtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgcgaacgccaccgcgtccaactcctctatcatcgaggggatgaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgagcaacaacacgcgcacctccatccggatcggccctggccaggccttctacgccaccggccaggtgatcggcgacatccgcgaggcgcactgcaacatctcggagtccaagtggaacgagaccctgcagcgcgtgtccgagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccacgtccaccgacatggccaactccaccgagaccaactccacgcgcatcatcacgatccggtgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcgagacgttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaagaattcggtgaccgggtcccggatcc

SEQ ID NO: 21 - DNA, 1520 bp, Artificial Sequence
  Description of Artificial Sequence Synthetic polynucleotide
ggtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccgacgccaccgcgtccaacgcaaccgcgagcaacgccacggcgtcgaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgtcgaacaacacgcgcacctccatccggatcggccctggccaggccttctacgccaccggccaggtgatcggcgacatccgcgaggcgcactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgagaccaactccacgcgcatcatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaacaacaacacggagaccttcaggccaggcggaggcaacatgaaggacaactggcgctccgagctgtacaagtacaaggtggtggaggtgaagcccctgggcgtggcacccaccaacgcccgcgagcgcgtcgtggagcgcgagaaggagtagtaagaattcggtgaccgggacctggatcc

SEQ ID NO: 22 - DNA, 1514 bp, Artificial Sequence
  Description of Artificial Sequence Synthetic polynucleotide
ggtcgacaagaagccaccatgcgcgtgacgggcatccagcgcaactacccgcagtggtggatctggtcgatgctgggcctctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggcggaccagatgcacgaggacgtgatctccctgtgggaccagtccctgaagccctgcgtgaagctgaccccgctgtgcgtgaccctgaactgcatcgacgccaacgcgaccgcgtccaacgcgacggcatccaactcgtccatcatcgaggggatgaagaactgctccttcaacatcacgacggagctgcgcgacaagatcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcggagaacatcacgaacagcgcgaagaccatcatcgtgcacctgaacgagtccgtgaagatcgagtgcacccgcccgagcaacaacacgcgcacctccatccggatcggccctggccaggccttctacgccaccggccaggtgatcggcgacatccgcaaggcgcactgcaacatctccgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgagaccaactccacgcgcacgatcacgctccactgccgcatcaagcagatcatcaacatgtggcaggaggtgggccgcgccatgtacgcaccgcccatcgccggcaacatcacctgcatctccaacatcaccggcctcctgctgacccgcgacggcggcaacaacaacaccacggagaccttcaggccagggggaggcaacatgaaggacaactggcgctccgagctgtacaagtacaaggtggtggagatcaagcccctgggcgtggcacccaccaacgcccgcgagcgcgtcgtggagcgcgagaaggagtagtaagaattcggtaaccaggtcccggatcc

SEQ ID NO: 23 - DNA, 1517 bp, Artificial Sequence
  Description of Artificial Sequence Synthetic polynucleotide
ggtcgacaagaagccaccatgcgcgtgatgggcatccagcgcaactacccgcagtggtggatctggtcgatgctgggcttctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgagctgcacgaacgccaccaacgcgacggcgtcgaactcgtccatcctcgaggggatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcgaagaacatcacggacaacgggaagaccatcatcgtgcacctgaacgagtccgtgaagatcgagtgcacccgcccgagcaacaacacgcgcacctccatccggatcggccctggccaggccttctacgccaccggccaggtgatcggcgacatccgcgaggcgcactgcaacatctccgagtccaagtggaacgagaccctgcagcgcgtgtccgagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccacctccaccgacatggccaactccaccgagaccaactccacgcgcatcatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaagaacgacacggacaccttcaggccagagggaggcaacatgaaggacaactggcgctccgagctgtacaagtacaaggtggtggaggtgaagcccctgggcgtggcacccaccaacgcccgcgagcgcgtcgtggagcgcgagaaggagtagtaagaattcggtcaccgggtcctggatcc

SEQ ID NO: 24 - DNA, 1559 bp, Artificial Sequence
  Description of Artificial Sequence Synthetic polynucleotide
ggtcgacaagaagccaccatgaaggtgcggggcatccagcgcaactacccgcagtggtggatctggtcgatgctgggcctctggatgctcatgatctgcaacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctggagaacgtgaccgagaacttcaacatgtggaagaacgacatggcggaccagatgcacgaggacgtgatctccctgtgggaccagtccctgaagccctgcgtgaagctgaccccgctgtgcgtgaccctgaactgcacggacgccaacgccaccgcgtcgaacaccaacgcgaccgcaagcaacatcaacgcgacggcgtcgaagtcctccatcatcgaggagatgaagaactgctccttcaacatcacgacggagctgcgcgacaagcgcgagaagaagtacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcggagaacatcacggacaacagcaagaccatcatcgtgcacctgaacgagtccgtgaagatcgagtgcacccgcccgagcaacaacacgcgcacctccatccggatcggccctggccaggccttctacgccaccggccaagtgatcggcgacatccgcgaggcgcactgcaacatctccgagtccaagtggaacgagaccctgcagcgcgtgtccaagaagctgaaggagtacttccccgacaagaacatcaccttccagccgtcgtccggcggcgaccccgagatcaccacgcactccttcaactgcggtggcgagttcttctactgcaacacgtcgtccctgttcaaccgcacctacatggcgaactccaccgagaccaactccacgcgcaccatcacgctgcactgccgcatcaagcagatcatcaacatgtggcaggaggtgggccgcgccatgtacgcaccgcccatcgccggcaacatcacctgcatctccaacatcaccggcctcctgctgacccgcgacggcggcgagaacacccgggacggaggcaacaacaacacggagaccttcaggccagagggaggcaacatgaaggacaactggcgctccgagctgtacaagtacaaggtggtggaggtgaagcccctgggcgtggcacccaccaaggcccgcgagcgcgtcgtggagcgcgagaaggagtagtaaggtaaccgggacccgaattcggatcc

SEQ ID NO: 25 - protein, 486 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNAKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVEVKPLGVAPTNARERVVEREKE

SEQ ID NO: 26 - protein, 486 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITDNGKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVEVKPLGVAPTNARERVVEREKE

SEQ ID NO: 27 - protein, 486 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNNSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNAKTIIVHLNESVKIECTRPNNKTRTSIRIGPGQAFYATGQVIGDIRKAYCNISESKWNETLQRVSKKLKEYFPHKNITFQPSSGGDLEITTHSFNCGGEFFYCNTSSLFNRTYMANSTDMANSTETNSTRTITLHCRIKQIINMWQEVGRAMYAPPIAGNITCISNITGLLLTRDGGKNNTETFRPGGGNMKDNWRSELYKYKVVEVKPLGVAPTNARERVVEREKE

SEQ ID NO: 28 - protein, 486 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKMTPLCVTLNCTNATAINSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNDKTIIVHLNESVKIECTRPNNKTRTSIRIGPGQAFYATGQVIGDIREAHCNISESKWNETLQRVSKKLKEYFPHKNITFQPSSGGDLEITTHSFNCGGEFFYCNTSSLFNRTYMANSTDMANSTETNSTRTITIRCRIKQIINMWQEVGRAMYAPPIAGNITCISNITGLLLTRDGGKNNTETFRPGGGNMKDNWRSELYKYKVVEVKPLGVAPTNARERVVEREKE

SEQ ID NO: 29 - protein, 490 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLNCTNATTNATASNSSIIEEMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNTAKTIIVHLNESVKIECTRPNNKTRTSIRIGPGQAFYATGQVIGDIREAYCNISESKWNETLQRVSKKLKEYFPHKNITFQPSSGGDLEITTHSFNCGGEFFYCNTSSLFNRTYMANSTDMANSTETNSTRTITIRCRIKQIINMWQEVGRAMYAPPIAGNITCISNITGLLLTRDGGENNTETFRPGGGNMKDNWRSELYKYKVVEVKPLGVAPTNARERVVEREKE

SEQ ID NO: 30 - protein, 499 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLNCTNANATASNSSIIEGMNSSIIEGMKNCSFNITTELRDKREKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITDNGKTIIVHLNESVKIECTRPSNNTRTSIRIGPGQAFYATGQVIGDIREAHCNISESKWNETLQRVSEKLKEYFPHKNITFQPSSGGDLEITTHSFNCGGEFFYCNTSSLFNRTYMATSTDMANSTETNSTRIITIRCRIKQIINMWQEVGRAMYAPPIAGNITCISNITGLLLTRDGGKNNTETFETFRPGGGNMKDNWRSELYKYKVVEVKPLGVAPTNARERVVEREKE

SEQ ID NO: 31 - protein, 490 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLNCTDATASNATASNATASNSSIIEGMKNCSFNITTELRDKREKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITDNGKTIIVHLNESVKIECTRPSNNTRTSIRIGPGQAFYATGQVIGDIREAHCNISESKWNETLQRVSKKLKEYFPHKNITFQPSSGGDLEITTHSFNCGGEFFYCNTSSLFNRTYMANSTETNSTRIITIRCRIKQIINMWQEVGRAMYAPPIAGNITCISNITGLLLTRDGGNNNTETFRPGGGNMKDNWRSELYKYKVVEVKPLGVAPTNARERVVEREKE

SEQ ID NO: 32 - protein, 488 aa, Artificial Sequence
  Description of Artificial Sequence Synthetic polypeptide
MRVTGIQRNYPQWWIWSMLGLWMLMICNGVPVWKEAKTTLFCASDAKAYEKEVHNVWATHACVPTDPNPQEMVLKNVTENFNMWKNDMADQMHEDVISLWDQSLKPCVKLTPLCVTLNCIDANATASNATASNSSIIEGMKNCSFNITTELRDKIEKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITNSAKTIIVHLNESVKIECTRPSNNTRTSIRIGPGQAFYATGQVIGDIRKAHCNISESKWNETLQRVSKKLKEYFPHKNITFQPSSGGDLEITTHSFNCGGEFFYCNTSSLFNRTYMANSTETNSTRTITLHCRIKQIINMWQEVGRAMYAPPIAGNITCISNITGLLLTRDGGNNNTTETFRPGGGNMKDNWRSELYKYKVVEIKPLGVAPTNARERVVEREKE

SEQ ID NO: 33 - protein, 489 aa, Artificial Sequence
  Description of Artificial Sequence Synthetic polypeptide
MRVMGIQRNYPQWWIWSMLGFWMLMICNGVPVWKEAKTTLFCASDAKAYEKEVHNVWATHACVPTDPNPQEMVLKNVTENFNMWKNDMVDQMHEDVISLWDQSLKPCVKLTPLCVTLSCTNATNATASNSSILEGMKNCSFNITTELRDKREKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSKNITDNGKTIIVHLNESVKIECTRPSNNTRTSIRIGPGQAFYATGQVIGDIREAHCNISESKWNETLQRVSEKLKEYFPHKNITFQPSSGGDLEITTHSFNCGGEFFYCNTSSLFNRTYMATSTDMANSTETNSTRIITIRCRIKQIINMWQEVGRAMYAPPIAGNITCISNITGLLLTRDGGKNDTDTFRPEGGNMKDNWRSELYKYKVVEVKPLGVAPTNARERVVEREKE

SEQ ID NO: 34 - protein, 503 aa, Artificial Sequence
  Description of Artificial Sequence Synthetic polypeptide
MKVRGIQRNYPQWWIWSMLGLWMLMICNGVPVWKEAKTTLFCASDAKAYEKEVHNVWATHACVPTDPNPQEMVLENVTENFNMWKNDMADQMHEDVISLWDQSLKPCVKLTPLCVTLNCTDANATASNTNATASNINATASKSSIIEEMKNCSFNITTELRDKREKKYALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITDNSKTIIVHLNESVKIECTRPSNNTRTSIRIGPGQAFYATGQVIGDIREAHCNISESKWNETLQRVSKKLKEYFPDKNITFQPSSGGDPEITTHSFNCGGEFFYCNTSSLFNRTYMANSTETNSTRTITLHCRIKQIINMWQEVGRAMYAPPIAGNITCISNITGLLLTRDGGENTRDGGNNNTETFRPEGGNMKDNWRSELYKYKVVEVKPLGVAPTKARERVVEREKE

SEQ ID NO: 35 - DNA, 2131 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgcgaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatcaacgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgaggtcaccgaattcgggacccggatcc

SEQ ID NO: 36 - DNA, 2131 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatcaacgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgaggtgaccgaattcaggtcccggatcc

SEQ ID NO: 37 - DNA, 2131 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaacaactccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgcgaagaccatcatcgtgcacctgaacgagtccgtgaagatcgagtgcacccgcccgaacaacaagacgcgcacgtccatccggatcggccctggccaggccttctacgccaccggccaggtgatcggcgacatccgcaaggcgtactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgctccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagggacccgaattcggtcaccggatcc

SEQ ID NO: 38 - DNA, 2131 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagatgaccccgctgtgcgtgaccctgaactgcaccaacgccaccgcgatcaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgacaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgcactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagagctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagggtcctgaattcggttaccggatcc

SEQ ID NO: 39 - DNA, 2143 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgcgacgacgaacgccaccgcgtccaactcgtccatcatcgaggagatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacacggcgaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccggtgccgcatcaagcagatcatcaacatgtggcaggaggtgggccgcgccatgtacgcaccgcccatcgccggcaacatcacctgcatctccaacatcaccggcctcctgctgacccgcgacggcggcgagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagggtcccgaattcggttaccggatcc

SEQ ID NO: 40 - DNA, 2170 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgcgaacgccaccgcgtccaactcctctatcatcgaggggatgaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgagcaacaacacgcgcacctccatccggatcggccctggccaggccttctacgccaccggccaggtgatcggcgacatccgcgaggcgcactgcaacatctcggagtccaagtggaacgagaccctgcagcgcgtgtccgagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccacgtccaccgacatggccaactccaccgagaccaactccacgcgcatcatcacgatccggtgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcgagacgttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagaattcggtgaccgggtcccggatcc

SEQ ID NO: 41 - DNA, 2143 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccgacgccaccgcgtccaacgcaaccgcgagcaacgccacggcgtcgaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgtcgaacaacacgcgcacctccatccggatcggccctggccaggccttctacgccaccggccaggtgatcggcgacatccgcgaggcgcactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgagaccaactccacgcgcatcatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaacaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcggggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatgatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagaattcggtgaccgggacctggatcc

SEQ ID NO: 42 - DNA, 2137 bp, Artificial Sequence
  Description of Artificial Sequence Synthetic polynucleotide
gtcgacaagaagccaccatgcgcgtgacgggcatccagcgcaactacccgcagtggtggatctggtcgatgctgggcctctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggcggaccagatgcacgaggacgtgatctccctgtgggaccagtccctgaagccctgcgtgaagctgaccccgctgtgcgtgaccctgaactgcatcgacgccaacgcgaccgcgtccaacgcgacggcatccaactcgtccatcatcgaggggatgaagaactgctccttcaacatcacgacggagctgcgcgacaagatcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcggagaacatcacgaacagcgcgaagaccatcatcgtgcacctgaacgagtccgtgaagatcgagtgcacccgcccgagcaacaacacgcgcacctccatccggatcggccctggccaggccttctacgccaccggccaggtgatcggcgacatccgcaaggcgcactgcaacatctccgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgagaccaactccacgcgcacgatcacgctccactgccgcatcaagcagatcatcaacatgtggcaggaggtgggccgcgccatgtacgcaccgcccatcgccggcaacatcacctgcatctccaacatcaccggcctcctgctgacccgcgacggcggcaacaacaacaccacggagaccttcaggccagggggaggcaacatgaaggacaactggcgctccgagctgtacaagtacaaggtggtggagatcaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccgactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagaattcggtaaccaggtcccggatcc

SEQ ID NO: 43 - DNA, 2140 bp, Artificial Sequence
  Description of Artificial Sequence Synthetic polynucleotide
gtcgacaagaagccaccatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgagctgcacgaacgccaccaacgcgacggcgtcgaactcgtccatcctcgaggggatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcgaagaacatcacggacaacgggaagaccatcatcgtgcacctgaacgagtccgtgaagatcgagtgcacccgcccgagcaacaacacgcgcacctccatccggatcggccctggccaggccttctacgccaccggccaggtgatcggcgacatccgcgaggcgcactgcaacatctccgagtccaagtggaacgagaccctgcagcgcgtgtccgagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccacctccaccgacatggccaactccaccgagaccaactccacgcgcatcatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaagaacgacacggacaccttcaggccagagggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagctgatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctgatgagaattcggtcaccgggtcctggatcc

SEQ ID NO: 44 - DNA, 2182 bp, Artificial Sequence
  Description of Artificial Sequence Synthetic polynucleotide
gtcgacaagaagccaccatgaaggtgcggggcatccagcgcaactacccgcagtggtggatctggtcgatgctgggcctctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctggagaacgtgaccgagaacttcaacatgtggaagaacgacatggcggaccagatgcacgaggacgtgatctccctgtgggaccagtccctgaagccctgcgtgaagctgaccccgctgtgcgtgaccctgaactgcacggacgccaacgccaccgcgtcgaacaccaacgcgaccgcaagcaacatcaacgcgacggcgtcgaagtcctccatcatcgaggagatgaagaactgctccttcaacatcacgacggagctgcgcgacaagcgcgagaagaagtacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcggagaacatcacggacaacagcaagaccatcatcgtgcacctgaacgagtccgtgaagatcgagtgcacccgcccgagcaacaacacgcgcacctccatccggatcggccctggccaggccttctacgccaccggccaagtgatcggcgacatccgcgaggcgcactgcaacatctccgagtccaagtggaacgagaccctgcagcgcgtgtccaagaagctgaaggagtacttccccgacaagaacatcaccttccagccgtcgtccggcggcgaccccgagatcaccacgcactccttcaactgcggtggcgagttcttctactgcaacacgtcgtccctgttcaaccgcacctacatggcgaactccaccgagaccaactccacgcgcaccatcacgctgcactgccgcatcaagcagatcatcaacatgtggcaggaggtgggccgcgccatgtacgcaccgcccatcgccggcaacatcacctgcatctccaacatcaccggcctcctgctgacccgcgacggcggcgagaacacccgggacggaggcaacaacaacacggagaccttcaggccagagggaggcaacatgaaggacaactggcgctccgagctgtacaagtacaaggtggtggaggtgaagcccctgggcgtggcacccaccaaggcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgacatcatctacgacctcctcgaggagtcccagaaccagcaggagaagaacgagcaggatctgctcgcgctggaccgctggaactccctgtggaactggttcaacatcaccaagtggctgtggtacatcaagatcttcatcatgatcgtgggcggcctgatcggcctgcgcatcatcttcgccgtgctgtcgctggtgaaccgcgtgcgccagggctgatgaggtaaccgggacccgaattcggatcc

SEQ ID NO: 45 - DNA, 2582 bp, Artificial Sequence
  Description of Artificial Sequence Synthetic polynucleotide
ggtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgcgaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatcaacgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggtgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaaggtcaccgaattcgggacccggatcc

SEQ ID NO: 46 - DNA, 2581 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatcaacgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggtgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaaggtgaccgaattcaggtcccggatcc

SEQ ID NO: 47 - DNA, 2581 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgccaccgcgtccaacaactccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgcgaagaccatcatcgtgcacctgaacgagtccgtgaagatcgagtgcacccgcccgaacaacaagacgcgcacgtccatccggatcggccctggccaggccttctacgccaccggccaggtgatcggcgacatccgcaaggcgtactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgctccactgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggtgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaagggacccgaattcggtcaccggatcc

SEQ ID NO: 48 - DNA, 2581 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagatgaccccgctgtgcgtgaccctgaactgcaccaacgccaccgcgatcaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacaacgacaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgcactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagagctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggtgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaagggtcctgaattcggttaccggatcc

SEQ ID NO: 49 - DNA, 2593 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgcgacgacgaacgccaccgcgtccaactcgtccatcatcgaggagatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaccggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacgaacacggcgaagaccatcatcgtgcacctgaacgagtccgtgaagatcgagtgcacccgcccgaacaacaagacgcgcacctccatccggatcggccctggccaggccttctacgccaccggccaggtgatcggcgacatccgcgaggcgtactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgacatggccaactccaccgagaccaactccacgcgcaccatcacgatccggtgccgcatcaagcagatcatcaacatgtggcaggaggtgggccgcgccatgtacgcaccgcccatcgccggcaacatcacctgcatctccaacatcaccggcctcctgctgacccgcgacggcggcgagaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggtgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaagggtcccgaattcggttaccggatcc

SEQ ID NO: 50 - DNA, 2620 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccaacgcgaacgccaccgcgtccaactcctctatcatcgaggggatgaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgagcaacaacacgcgcacctccatccggatcggccctggccaggccttctacgccaccggccaggtgatcggcgacatccgcgaggcgcactgcaacatctcggagtccaagtggaacgagaccctgcagcgcgtgtccgagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccacgtccaccgacatggccaactccaccgagaccaactccacgcgcatcatcacgatccggtgccgcatcaagcagatcatcaacatgtggcaggaggtgggccgcgccatgtacgcaccgcccatcgccggcaacatcacctgcatctccaacatcaccggcctcctgctgacccgcgacggcggcaagaacaacacggagaccttcgagacgttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggcgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaagaattcggtgaccgggtcccggatcc

SEQ ID NO: 51 - DNA, 2593 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgaactgcaccgacgccaccgcgtccaacgcaaccgcgagcaacgccacggcgtcgaactcctccatcatcgagggcatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtccgagaacatcacggacaacgggaagaccatcatcgtgcacctgaacgagtccgtgaagatcgagtgcacccgcccgtcgaacaacacgcgcacctccatccggatcggccctggccaggccttctacgccaccggccaggtgatcggcgacatccgcgaggcgcactgcaacatctcggagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgagaccaactccacgcgcatcatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaacaacaacacggagaccttcaggccaggcggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcggggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagatgatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaagcgctccacgcgcctggtgtccggcttcctggccctggtgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggctcgctggtgcagtactggggcctggagctgaagcgctccgccatctccctgctggacaccctggccatcgccgtgggcgagggcaccgaccgcatcctggagttcgcgctgggcatctgccgcgccatccgcaacatccccacccgcatccgccagggcttcgagaccgccctcctgtagtaagaattcggtgaccgggacctggatcc

SEQ ID NO: 52 - DNA, 2587 bp, Artificial Sequence
  Description of Artificial Sequence Synthetic polynucleotide
gtcgacaagaaatgcgcgtgacgggcatccagcgcaactacccgcagtggtggatctggtcgatgctgggcctctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggcggaccagatgcacgaggacgtgatctccctgtgggaccagtccctgaagccctgcgtgaagctgaccccgctgtgcgtgaccctgaactgcatcgacgccaacgcgaccgcgtccaacgcgacggcatccaactcgtccatcatcgaggggatgaagaactgctccttcaacatcacgacggagctgcgcgacaagatcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcggagaacatcacgaacagcgcgaagaccatcatcgtgcacctgaacgagtccgtgaagatcgagtgcacccgcccgagcaacaacacgcgcacctccatccggatcggccctggccaggccttctacgccaccggccaggtgatcggcgacatccgcaaggcgcactgcaacatctccgagtccaagtggaacgagaccctgcagcgcgtgtccaagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccaactccaccgagaccaactccacgcgcacgatcacgctccactgccgcatcaagcagatcatcaacatgtggcaggaggtgggccgcgccatgtacgcaccgcccatcgccggcaacatcacctgcatctccaacatcaccggcctcctgctgacccgcgacggcggcaacaacaacaccacggagaccttcaggccagggggaggcaacatgaaggacaactggcgctccgagctgtacaagtacaaggtggtggagatcaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccgactacaccgagatcatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgacgccctccccccggggcccggacaggcccggtggcatcgaggaggagggcggcgagcaggaccgcaaccgctccacgcgcctggtgtccggcttcctggccctggcgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggcggcctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaagaattcggtaaccaggtcccggatcc

SEQ ID NO: 53 - DNA, 2590 bp, Artificial Sequence
  Description of Artificial Sequence Synthetic polynucleotide
gtcgacaagaaatgcgcgtgatgggcatccagcgcaactacccgcagtggtggatctggtcgatgctgggcttctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctgaagaacgtgaccgagaacttcaacatgtggaagaacgacatggtggaccagatgcacgaggacgtgatctccctgtgggaccagtccctgaagccctgcgtgaagctgaccccgctgtgcgtgaccctgagctgcacgaacgccaccaacgcgacggcgtcgaactcgtccatcctcgaggggatgaagaactgctccttcaacatcacgacggagctgcgcgacaagcgcgagaagaagaacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcgaagaacatcacggacaacgggaagaccatcatcgtgcacctgaacgagtccgtgaagatcgagtgcacccgcccgagcaacaacacgcgcacctccatccggatcggccctggccaggccttctacgccaccggccaggtgatcggcgacatccgcgaggcgcactgcaacatctccgagtccaagtggaacgagaccctgcagcgcgtgtccgagaagctgaaggagtacttcccccacaagaacatcaccttccagccgtcgtccggcggcgacctcgagatcaccacgcactccttcaactgcggtggcgagttcttctactgcaacacgtcgtcgctgttcaaccgcacctacatggccacctccaccgacatggccaactccaccgagaccaactccacgcgcatcatcacgatccgctgccgcatcaagcagatcatcaacatgtggcaggaggtgggccgcgccatgtacgcaccgcccatcgccggcaacatcacctgcatctccaacatcaccggcctcctgctgacccgcgacggcggcaagaacgacacggacaccttcaggccagagggaggcaacatgaaggacaactggcgctccgagctgtacaagtacaaggtggtggaggtgaagcccctgggcgtggcacccaccaacgcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgagctgatctacgagctcctcgaggagtcccagaaccagcaggagaagaacgagcaggatctgctcgcgctggaccgctggaactccctgtggaactggttcaacatcaccaactggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggcgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggcaacctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaagaattcggtcaccgggtcctggatcc

SEQ ID NO: 54 - DNA, 2632 bp, Artificial Sequence
  Description of Artificial Sequence Synthetic polynucleotide
gtcgacaagaaatgaaggtgcggggcatccagcgcaactacccgcagtggtggatctggtcgatgctgggcctctggatgctcatgatctgcaacggcatgtgggtgacggtgtactacggcgtgccggtgtggaaggaggccaagacgaccctgttctgcgcgtcggacgccaaggcctacgagaaggaggtgcacaacgtgtgggcgacccacgcctgcgtgcccacggaccccaacccgcaggagatggtgctggagaacgtgaccgagaacttcaacatgtggaagaacgacatggcggaccagatgcacgaggacgtgatctccctgtgggaccagtccctgaagccctgcgtgaagctgaccccgctgtgcgtgaccctgaactgcacggacgccaacgccaccgcgtcgaacaccaacgcgaccgcaagcaacatcaacgcgacggcgtcgaagtcctccatcatcgaggagatgaagaactgctccttcaacatcacgacggagctgcgcgacaagcgcgagaagaagtacgccctgttctacaagctggacatcgtgcagctggacggcaactcctcgcagtacaggctgatcaactgcaacacctccgtcatcacgcaggcgtgccccaaggtgtccttcgaccccatccccatccactactgcgcccccgccggctacgccatcctgaagtgcaacaacaagaccttcaacggcaccggcccgtgcaacaacgtgtccaccgtgcagtgcacgcacgggatcaagcccgtggtgtccacgcagctgctcctgaacgggtcgctggccgagggcgagatcatcatccggtcggagaacatcacggacaacagcaagaccatcatcgtgcacctgaacgagtccgtgaagatcgagtgcacccgcccgagcaacaacacgcgcacctccatccggatcggccctggccaggccttctacgccaccggccaagtgatcggcgacatccgcgaggcgcactgcaacatctccgagtccaagtggaacgagaccctgcagcgcgtgtccaagaagctgaaggagtacttccccgacaagaacatcaccttccagccgtcgtccggcggcgaccccgagatcaccacgcactccttcaactgcggtggcgagttcttctactgcaacacgtcgtccctgttcaaccgcacctacatggcgaactccaccgagaccaactccacgcgcaccatcacgctgcactgccgcatcaagcagatcatcaacatgtggcaggaggtgggccgcgccatgtacgcaccgcccatcgccggcaacatcacctgcatctccaacatcaccggcctcctgctgacccgcgacggcggcgagaacacccgggacggaggcaacaacaacacggagaccttcaggccagagggaggcaacatgaaggacaactggcgctccgagctgtacaagtacaaggtggtggaggtgaagcccctgggcgtggcacccaccaaggcccgcaggcgcgtcgtggagcgcgagaagcgcgccgtgggcatgggcgccgtgttcctgggcttcctgggcgctgcgggctccaccatgggtgccgcgtccatcaccctgaccgtgcaggcccgccagctgctctccggcatcgtgcagcagcagtccaacctcctgaaggccatcgaggcccagcagcacatgctgaagctgaccgtgtggggcatcaagcagctgcaggccagggtgctcgcgctcgagcgctacctgaaggaccagcagctgctcggcatgtggggctgctccggcaagctgatctgcaccaccaacgtgtactggaactcgtcctggtccaacaagacctacggcgacatctgggacaacatgacctggatgcagtgggagcgcgagatctccaactacaccgacatcatctacgacctcctcgaggagtcccagaaccagcaggagaagaacgagcaggatctgctcgcgctggaccgctggaactccctgtggaactggttcaacatcaccaagtggctgtggtacatcaagatcttcatcatgatcgtgggcggcctgatcggcctgcgcatcatcttcgccgtgctgtcgctggtgaaccgcgtgcgccagggctactccccgctgtccctgcaaacgctgatcccctccccccggggcccggacaggcccggtggcatcgaggaggagggcggcgagcaggaccgcaaccgctccacgcgcctggtgtccggcttcctggccctggcgtgggacgacctgcgctccctgtgcctgttcatctaccaccgcctgcgcgacttcatcctgatcgcggcccgcgctggcgagctgctgggccggtcctcgctgaagggcctgcgccgcggctgggaggccctgaagtacctgggcgggctggtgcagtactggggcctggagctgaagcgctccgccatctccctgctggacaccctggccatcgccgtgggcgagggcaccgaccgcatcctggagttcgtgctgggcatctgccgcgccatccgcaacatccccacccgcatccgccagggcttcgagaccgccctcctgtagtaaggtaaccgggacccgaattcggatcc

SEQ ID NO: 55 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNAKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 56 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITDNGKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 57 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNNSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNAKTIIVHLNESVKIECTRPNNKTRTSIRIGPGQAFYATGQVIGDIRKAYCNISESKWNETLQRVSKKLKEYFPHKNITFQPSSGGDLEITTHSFNCGGEFFYCNTSSLFNRTYMANSTDMANSTETNSTRTITLHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 58 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKMTPLCVTLNCTNATAINSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNDKTIIVHLNESVKIECTRPNNKTRTSIRIGPGQAFYATGQVIGDIREAHCNISESKWNETLQRVSKKLKEYFPHKNITFQPSSGGDLEITTHSFNCGGEFFYCNTSSLFNRTYMANSTDMANSTETNSTRTITIRCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKSYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 59 - protein, 850 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATTNATASNSSIIEEMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNTAKTIIVHLNESVKIECTRPNNKTRTSIRIGPGQAFYATGQVIGDIREAYCNISESKWNETLQRVSKKLKEYFPHKNITFQPSSGGDLEITTHSFNCGGEFFYCNTSSLFNRTYMANSTDMANSTETNSTRTITIRCRIKQIINMWQEVGRAMYAPPIAGNITCISNITGLLLTRDGGENNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 60 - protein, 859 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNANATASNSSIIEGMNSSIIEGMKNCSFNITTELRDKREKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITDNGKTIIVHLNESVKIECTRPSNNTRTSIRIGPGQAFYATGQVIGDIREAHCNISESKWNETLQRVSEKLKEYFPHKNITFQPSSGGDLEITTHSFNCGGEFFYCNTSSLFNRTYMATSTDMANSTETNSTRIITIRCRIKQIINMWQEVGRAMYAPPIAGNITCISNITGLLLTRDGGKNNTETFETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALAWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 61 - protein, 850 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTDATASNATASNATASNSSIIEGMKNCSFNITTELRDKREKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITDNGKTIIVHLNESVKIECTRPSNNTRTSIRIGPGQAFYATGQVIGDIREAHCNISESKWNETLQRVSKKLKEYFPHKNITFQPSSGGDLEITTHSFNCGGEFFYCNTSSLFNRTYMANSTETNSTRIITIRCRIKQIINMWQEVGRAMYAPPIAGNITCISNITGLLLTRDGGNNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIGAQQHMLKLTVWGIKQLQARVLALERYLKDQQLLGMWGCSGKLICTTNVYWNSSWSNKTYGDIWDNMTWMQWEREISNYTEMIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRKRSTRLVSGFLALVWDDLRSLCLFIYHRLRDFILIAARAGELLGRSSLKGLRRGWEALKYLGSLVQYWGLELKRSAISLLDTLAIAVGEGTDRILEFALGICRAIRNIPTRIRQGFETALL

SEQ ID NO: 62 - protein, 848 aa, Artificial Sequence
  Description of Artificial Sequence Synthetic polypeptide
MRVTGIQRNYPQWWIWSMLGLWMLMICNGMWVTVYYGVPVWKEAKTTLFCASDAKAYEKEVHNVWATHACVPTDPNPQEMVLKNVTENFNMWKNDMADQMHEDVISLWDQSLKPCVKLTPLCVTLNCIDANATASNATASNSSIIEGMKNCSFNITTELRDKIEKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITNSAKTIIVHLNESVKIECTRPSNNTRTSIRIGPGQAFYATGQVIGDIRKAHCNISESKWNETLQRVSKKLKEYFPHKNITFQPSSGGDLEITTHSFNCGGEFFYCNTSSLFNRTYMANSTETNSTRTITLHCRIKQIINMWQEVGRAMYAPPIAGNITCISNITGLLLTRDGGNNNTTETFRPGGGNMKDNWRSELYKYKVVEIKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISDYTEIIYELLEESQNQQEKNEQDLLALDRWNSLWNWFNITNWLWYIKIFIMIVGGLIGLRIIFAVLSLVNRVRQGYSPLSLQTLTPSPRGPDRPGGIEEEGGEQDRNRSTRLVSGFLALAWDDLRSLCLFIYHRLRDFILIAARAGELLGRSSLKGLRRGWEALKYLGGLVQYWGLELKRSAISLLDTLAIAVGEGTDRILEFVLGICRAIRNIPTRIRQGFETALL

SEQ ID NO: 63 - protein, 849 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLSCTNATNATASNSSILEGMKNCSFNITTELRDKREKKNALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSKNITDNGKTIIVHLNESVKIECTRPSNNTRTSIRIGPGQAFYATGQVIGDIREAHCNISESKWNETLQRVSEKLKEYFPHKNITFQPSSGGDLEITTHSFNCGGEFFYCNTSSLFNRTYMATSTDMANSTETNSTRIITIRCRIKQIINMWQEVGRAMYAPPIAGNITCISNITGLLLTRDGGKNDTDTFRPEGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTELIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALAWDDLRSLCLFIYHRLRDFILIAARAGELLGRSSLKGLRRGWEALKYLGNLVQYWGLELKRSAISLLDTLAIAVGEGTDRILEFVLGICRAIRNIPTRIRQGFETALL

SEQ ID NO: 64 - protein, 863 aa, Artificial Sequence
  Description of Artificial Sequence Synthetic polypeptide
MKVRGIQRNYPQWWIWSMLGLWMLMICNGMWVTVYYGVPVWKEAKTTLFCASDAKAYEKEVHNVWATHACVPTDPNPQEMVLENVTENFNMWKNDMADQMHEDVISLWDQSLKPCVKLTPLCVTLNCTDANATASNTNATASNINATASKSSIIEEMKNCSFNITTELRDKREKKYALFYKLDIVQLDGNSSQYRLINCNTSVITQACPKVSFDPIPIHYCAPAGYAILKCNNKTFNGTGPCNNVSTVQCTHGIKPVVSTQLLLNGSLAEGEIIIRSENITDNSKTIIVHLNESVKIECTRPSNNTRTSIRIGPGQAFYATGQVIGDIREAHCNISESKWNETLQRVSKKLKEYFPDKNITFQPSSGGDPEITTHSFNCGGEFFYCNTSSLFNRTYMANSTETNSTRTITLHCRIKQIINMWQEVGRAMYAPPIAGNITCISNITGLLLTRDGGENTRDGGNNNTETFRPEGGNMKDNWRSELYKYKVVEVKPLGVAPTKARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTDIIYDLLEESQNQQEKNEQDLLALDRWNSLWNWFNITKWLWYIKIFIMIVGGLIGLRIIFAVLSLVNRVRQGYSPLSLQTLIPSPRGPDRPGGIEEEGGEQDRNRSTRLVSGFLALAWDDLRSLCLFIYHRLRDFILIAARAGELLGRSSLKGLRRGWEALKYLGGLVQYWGLELKRSAISLLDTLAIAVGEGTDRILEFVLGICRAIRNIPTRIRQGFETALL

SEQ ID NO: 65 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 66 - protein, 859 aa, Artificial Sequence; identical to SEQ ID NO: 60
  Description of Artificial Sequence Synthetic polypeptide

SEQ ID NO: 67 - protein, 848 aa, Artificial Sequence; identical to SEQ ID NO: 62
  Description of Artificial Sequence Synthetic polypeptide

SEQ ID NO: 68 - protein, 863 aa, Artificial Sequence; identical to SEQ ID NO: 64
  Description of Artificial Sequence Synthetic polypeptide

SEQ ID NO: 69 - protein, 845 aa, Artificial Sequence
  Description of Artificial Sequence Synthetic polypeptide
MRVRGIPRNWPQWWIWGILGFWVIITCRVVGQLWVTVYYGVPVWTEAKTTLFCASDAKAYDKEVHNVWATHACVPTDPNPQEIVLGNITENFNMWENDMVDQMHEDIISLWDQSLKPCVKLTPLCVTLICTNANISVTSVSNDSRILNEEIKNCSFNTTTEIRDKKQQVYALFYRSDVAPLSNSSEYILINCNTSTITQACPKVTFDPIPLHYCAPAGYAILKCNNKTFNGTGPCLNVSTVQCTHGIKPVVSTQLLLNGSLAEEEIIIKSKNLTDDTNTIIVHLNKSIEIVCTRPGNNTRKSIRIGPGQTFFATGDIIGDIREAHCNLSKDAWNTTLEQIKRKLKEHFSSKEIKFAPSSGGDPEVATHSFNCRGEFFYCNTTKLFNENYTLSNNSNETIILPCRIKQIINMWQGVGRAMYAPPIAGNITCNSSITGLLLTRDKDSNTETFRPTGGNMKDNWRNELYKYKVVEIKPLGVAPTNAKRRVVEREKRAVGIGAVLLGFLGAAGSTMGAASITLTVQARQLLSGIVQQQSNLLRAIEAQQHMLQLTVWGIKQLQTRVLAIERYLKDQQLLGLWGCSGRLICTTNVPWNSSWSNKSQEDIWGNMTWMQWDREISNYTSTIYRLLEDSQNQQEKNEKDLLALDSWKNLWNWFDITKWLWYIKIFIIIVGGLIGLRIVLGVLAIVKRVRQGYSPLSFQTLIPSPRGLDRLGRIEEEGGEQDKDRSIRLVNGFLALAWEDLRNLCLFSYHQLRDFILIVARAVELLGRSSLRGLQRGWEALKYLGALIQQGGLELKKSAISLLDTTAIAVAEGTDRILELIQRICRAIRNIPTRIRQGFEAALL

SEQ ID NO: 70 - DNA, 2538 bp, Artificial Sequence
  Description of Artificial Sequence Synthetic polynucleotide
atgagagtgagggggataccgaggaattggccacaatggtggatatggggcatcttaggcttttgggtgataataacttgtagggtggtagggcaattgtgggtcacagtctattatggggtacctgtgtggacagaagcaaaaactactctattctgtgcatcagatgctaaagcatatgacaaagaagtgcataatgtctgggctacacatgcctgtgtacccacagaccccaatccacaagaaatagtcttgggaaatataacagaaaattttaacatgtgggaaaatgacatggtggatcagatgcatgaggatataatcagtttatgggatcaaagtctaaaaccatgtgtaaagttgactccactctgtgtcactttaatttgtacaaatgcaaatattagtgttaccagtgtgagtaatgatagcaggattttgaatgaagaaataaaaaattgctctttcaatacaaccacagaaataagagataagaaacagcaagtgtatgcacttttttatagatctgatgtagcaccacttagtaattctagtgagtatatattaataaattgtaatacctcaaccataacacaagcctgtccaaaggtcacttttgacccaattcctttgcattattgtgctccagctggttatgcgattctaaaatgcaacaataagacattcaatgggacaggaccatgccttaatgtcagcacagtacaatgtacacatggaattaagccagtggtatcaactcaattactgttaaatggtagcctagcagaagaggagataataattaaatctaaaaatctgacagatgataccaatacaataatagtacaccttaataaatctatagaaattgtgtgtacaagacctggcaataatacaagaaaaagtataaggataggaccaggacaaacattctttgcaacaggtgacataataggagacataagagaagcccattgtaaccttagtaaagatgcatggaacacaactctagaacagataaaaagaaaactcaaagaacacttctctagtaaagaaataaaatttgctccaagctcaggaggagatccagaagttgcgacacatagttttaattgtagaggagaatttttctattgcaatacaacaaaactgtttaatgagaattatacactgagcaataacagtaatgaaacaatcatactcccatgtagaataaaacaaattataaatatgtggcagggggtaggacgagcaatgtatgcccctcccattgcaggaaacataacatgtaactcaagtatcacaggactgttattgacgcgtgacaaagacagcaacacagagacattcagacctacaggaggaaatatgaaggacaattggagaaatgaattatacaaatataaagtggtagaaattaaaccattaggagtagcacccactaatgcaaaaaggagagtggtggagagagaaaaaagagcagtaggaataggagctgtgctccttgggttcttgggagcagcaggaagcactatgggcgcggcgtcaataacgctgacggtacaggccagacaactgttatctggtatagtgcaacagcaaagtaatttgctgagagctatagaggcgcaacagcacatgttgcaactcacagtctggggcattaaacagctccagacaagagtcttggctatagaaagatacctaaaggatcaacagctcctaggactttggggctgctctggaagactcatctgcaccactaatgtgccttggaactcgagttggagtaataaatctcaagaagatatttgggggaacatgacctggatgcagtgggatagagaaattagtaattacacaagcacaatatacaggttacttgaagactcgcaaaaccagcaggagaaaaatgaaaaggatttgttagcattggacagttggaaaaatctgtggaattggtttgacataacaaaatggctgtggtatataaaaatattcatcataatagtaggaggtttaataggtttgagaatagttttgggtgtgcttgctatagtaaaaagagttaggcagggatactcacctttgtcgtttcagacccttatcccaagtccgaggggactcgacaggctcggaagaatcgaagaagaaggtggagagcaagacaaagacagatccattcgattagtgaacggattcttagcacttgcttgggaagatctgcggaatctgtgcctcttcagctaccaccaattgagagactttatattgattgtagcgagagcagtggaacttctgggacgcagcagcctcaggggactacagagggggtgggaagctcttaagtatctgggagctcttatacagcaggggggtctggaactaaagaagagtgctattagtctgcttgataccacagcaatagcagtagctgaaggaacagataggattctagaattaatacaaagaatttgtagagctatccgcaacatacctacaagaataagacaaggctttgaagcagctttgctataa

SEQ ID NO: 71 - protein, 846 aa, Artificial Sequence; identical to SEQ ID NO: 56
  Description of Artificial Sequence Synthetic polypeptide

SEQ ID NO: 72 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTYMANSTDMANSTETNSTRTITIHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLGYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 73 - protein, 846 aa, Artificial Sequence; identical to SEQ ID NO: 57
  Description of Artificial Sequence Synthetic polypeptide

SEQ ID NO: 74 - protein, 846 aa, Artificial Sequence; identical to SEQ ID NO: 58
  Description of Artificial Sequence Synthetic polypeptide

SEQ ID NO: 75 - protein, 849 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNDKTIIVHLNESVKIECTRPSNKTRTSIRIGPGQAFYATGQVIGDIREAYCNISESKWNETLQRVSKKLKEYFPHKNITFQPSSGGDLEITTHSFNCGGEFFYCNTSSLFNRTYMANSTDMANSTETNSTRNITIHCRIKQIINMWQEVGRAMYAPPIAGNITCISNITGLLLTRDGGKNDTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFILGICRAIRNIPTRIRQGFETALL

SEQ ID NO: 76 - protein, 840 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATANNSNIIEEMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNVKTIIVHLNESVKIECTRPNNKTRTSIRIGPGQAFYATGQVIGDIREAYCNINESKWNETLQRVSKKLKEYFPHKNITFQPSSGGDLEITTHSFNCGGEFFYCNTSSLFNRTDMANSTETNNTRTITIHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 77 - protein, 846 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATTSNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNNDKTIIVHLNESVKIECTRPNNNTRTSIRIGPGQAFYATGQVIGDIREAYCNISESKWNETLQRVSKKLKEYFPHKNITFQPSSGGDLEITTHSFNCGGEFFYCNTSSLFNRTYMANSTDMANSTETNSTRIITIHCRIKQIINMWQEVGRAMYAPPIAGNITCISNITGLLLTRDGGKNNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVLSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 78 - protein, 849 aa, Artificial Sequence; identical to SEQ ID NO: 63
  Description of Artificial Sequence Synthetic polypeptide

SEQ ID NO: 79 - protein, 854 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQEMVLKNVTENFNMWKNDMVDQMHEDVISLWDQSLKPCVKLTPLCVTLNCTNATARNCTNATASNSSIIEGMKNCSFNITTELRDKREKKNALFYKLDIVQLDGNSSQYRLINCNTSVITQACPKVSFDPIPIHYCAPAGYAILKCNNKTFTGTGPCNNVSTVQCTHGIKPVVSTQLLLNGSLAEGEIIIRSENITNSGKTIIVHLNESVKIECTRPNNKTRTSIRIGPGQAFYATGQVIGDIREAYCNISESKWNETLQRVSKKLKEYFPHKNITFQPSSGGDLEITTHSFNCGGEFFYCNTSSLFNRTYMANSTDMANSTETNSTRIITIHCRIKQIINMWQEVGRAMYAPPIAGNITCISSITGLLLTRDGGENNTETFRPGGGNMKDNWRSELYKYKVVEVKPLGVAPTNARRRVVEREKRAVGMGAVFLGFLGAAGSTMGAASITLTVQARQLLSGIVQQQSNLLKAIEAQQHMLKLTVWGIKQLQARVLALERYLKDQQLLGMWGCSGKLICTTNVYWNSSWSNKTYGDIWDNMTWMQWEREISNYTEIIYELLEESQNQQEKNEQDLLALDRWNSLWNWFNITNWLWYIKIFIMIVGGLIGLRIIFAVFSLVNRVRQGYSPLSLQTLIPSPRGPDRPGGIEEEGGEQDRNRSTRLVSGFLALVWDDLRSLCLFIYHRLRDFILIAARAGELLGRSSLKGLRRGWEALKYLGSLVQYWGLELKRSAISLLDTLAIAVGEGTDRILEFVLGICRAIRNIPTRIRQGFETALL

SEQ ID NO: 80 - protein, 850 aa, Artificial Sequence; identical to SEQ ID NO: 59
  Description of Artificial Sequence Synthetic polypeptide

SEQ ID NO: 81 - protein, 18 aa, Artificial Sequence
  Description of Artificial Sequence Synthetic peptide
LRDKKQKVHALFYKADAV

SEQ ID NO: 82 - protein, 6 aa, Artificial Sequence
  Description of Artificial Sequence Synthetic 6xHis tag
HHHHHH

SEQ ID NO: 83 - protein, 10 aa, Artificial Sequence
  Description of Artificial Sequence Synthetic 10xHis tag
HHHHHHHHHH

SEQ ID NO: 84 - protein, 79 aa, Artificial Sequence
  Description of Artificial Sequence Synthetic polypeptide
MRVMGIQRNYPQWWIWSMLGFWMLMICNGMWVTVYYGVPVWKEAKTTLFCASDAKAYEKEVHNVWATHACVPTDPNPQE

SEQ ID NO: 85 - protein, 33 aa, Artificial Sequence
  Description of Artificial Sequence Synthetic polypeptide
TRPNNNTRKSIRIGPGQTFYATGDIIGNIRQAH

SEQ ID NO: 86 - DNA, 20 bp, Artificial Sequence
  Description of Artificial Sequence Synthetic primer
tttttttttttttttttttt

SEQ ID NO: 87 - DNA, 32 bp, Artificial Sequence
  Description of Artificial Sequence Synthetic oligonucleotide
tagtaaggtcaccgaattcgggacccggatcc

SEQ ID NO: 88 - DNA, 32 bp, Artificial Sequence
  Description of Artificial Sequence Synthetic oligonucleotide
tagtaaggtgaccgaattcaggtcccggatcc

SEQ ID NO: 89 - DNA, 32 bp, Artificial Sequence
  Description of Artificial Sequence Synthetic oligonucleotide
tagtaagggacccgaattcggtcaccggatcc

SEQ ID NO: 90 - DNA, 32 bp, Artificial Sequence
  Description of Artificial Sequence Synthetic oligonucleotide
tagtaagggtcctgaattcggttaccggatcc

SEQ ID NO: 91 - DNA, 32 bp, Artificial Sequence
  Description of Artificial Sequence Synthetic oligonucleotide
tagtaagggtcccgaattcggttaccggatcc

SEQ ID NO: 92 - DNA, 32 bp, Artificial Sequence
  Description of Artificial Sequence Synthetic oligonucleotide
tagtaagaattcggtgaccgggtcccggatcc

SEQ ID NO: 93 - DNA, 32 bp, Artificial Sequence
  Description of Artificial Sequence Synthetic oligonucleotide
tagtaagaattcggtgaccgggacctggatcc

SEQ ID NO: 94 - DNA, 32 bp, Artificial Sequence
  Description of Artificial Sequence Synthetic oligonucleotide
tagtaagaattcggtaaccaggtcccggatcc

SEQ ID NO: 95 - DNA, 32 bp, Artificial Sequence
  Description of Artificial Sequence Synthetic oligonucleotide
tagtaagaattcggtcaccgggtcctggatcc

SEQ ID NO: 96 - DNA, 32 bp, Artificial Sequence
  Description of Artificial Sequence Synthetic oligonucleotide
tagtaaggtaaccgggacccgaattcggatcc

SEQ ID NO: 97 - DNA, 32 bp, Artificial Sequence
  Description of Artificial Sequence Synthetic oligonucleotide
tgatgaggtcaccgaattcgggacccggatcc

SEQ ID NO: 98 - DNA, 32 bp, Artificial Sequence
  Description of Artificial Sequence Synthetic oligonucleotide
tgatgaggtgaccgaattcaggtcccggatcc

SEQ ID NO: 99 - DNA, 32 bp, Artificial Sequence
  Description of Artificial Sequence Synthetic oligonucleotide
tgatgagggacccgaattcggtcaccggatcc

SEQ ID NO: 100 - DNA, 32 bp, Artificial Sequence
  Description of Artificial Sequence Synthetic oligonucleotide
tgatgagggtcctgaattcggttaccggatcc

SEQ ID NO: 101 - DNA, 32 bp, Artificial Sequence
  Description of Artificial Sequence Synthetic oligonucleotide
tgatgagggtcccgaattcggttaccggatcc

SEQ ID NO: 102 - DNA, 32 bp, Artificial Sequence
  Description of Artificial Sequence Synthetic oligonucleotide
tgatgagaattcggtgaccgggtcccggatcc

SEQ ID NO: 103 - DNA, 32 bp, Artificial Sequence
  Description of Artificial Sequence Synthetic oligonucleotide
tgatgagaattcggtgaccgggacctggatcc

SEQ ID NO: 104 - DNA, 32 bp, Artificial Sequence
  Description of Artificial Sequence Synthetic oligonucleotide
tgatgagaattcggtaaccaggtcccggatcc

SEQ ID NO: 105 - DNA, 32 bp, Artificial Sequence
  Description of Artificial Sequence Synthetic oligonucleotide
tgatgagaattcggtcaccgggtcctggatcc

SEQ ID NO: 106 - DNA, 32 bp, Artificial Sequence
  Description of Artificial Sequence Synthetic oligonucleotide
tgatgaggtaaccgggacccgaattcggatcc

SEQ ID NO: 107 - protein, 35 aa, Human immunodeficiency virus
ITDEVRNCSFNMTTELRDKKQKVHALFYKLDIVPI

SEQ ID NO: 108 - protein, 30 aa, Human immunodeficiency virus
EINCTRPNNNTRPGEIIGDIRQAHCNISRA